(19) United States
(12) Patent Application Publication
(10) Pub. No.: US 2024/0093293 A1
(43) Pub. Date: Mar. 21, 2024

(54) METHODS FOR INCREASING MONOCLONAL NUCLEIC ACID AMPLIFICATION PRODUCTS

(71) Applicant: Singular Genomics Systems, Inc., San Diego, CA (US)

(72) Inventors: Timothy Looney, Austin, TX (US); Christian Berrios, San Diego, CA (US)

(21) Appl. No.: 18/459,086

(22) Filed: Aug. 31, 2023

Related U.S. Application Data
(60) Provisional application No. 63/403,276, filed on Sep. 1, 2022.

(51) Int. Cl. C12Q 1/6874; C12Q 1/6844

(52) U.S. Cl. CPC C12Q 1/6874; C12Q 1/6844

(57) ABSTRACT

Disclosed herein, inter alia, are methods and compositions useful for increasing monoclonal nucleic acid amplification products on a solid support.

### CROSS-REFERENCES TO RELATED APPLICATIONS

This application claims the benefit of U.S. Provisional Application No. 63/403,276, filed Sep. 1, 2022, which is incorporated herein by reference in its entirety and for all purposes.

### BACKGROUND

Genetic analysis is taking on increasing importance in modem society as a diagnostic, prognostic, or forensic tool. Next generation sequencing (NGS) methods often rely on the amplification of genomic fragments hybridized to polynucleotide primers on a solid surface. Ideally these amplification sites have one initial polynucleotide fragment which is amplified to generate a plurality of identical fragments, or complements thereof. However instances of polyclonal sites, (i.e. where more than one distinct polynucleotide is initially present and amplified) are common and negatively impact sequencing results by increasing sequencing duplications or producing simultaneous and interfering signaling. Furthermore, a potential complication of commercial cluster amplification techniques is that they form a random pattern of clusters on the surface. Thus there is a need in in the art to improve nucleic acid amplification techniques. Disclosed herein, inter alia, are solutions to these and other problems in the art.

### BRIEF SUMMARY

In an aspect is provided a method of amplifying a template polynucleotide on a solid support, the method including: a) hybridizing the template polynucleotide to a first oligonucleotide, wherein the first oligonucleotide includes a cleavable site and is attached to the solid support, and extending with a polymerase the first oligonucleotide to generate an immobilized complement of the template polynucleotide; b) denaturing the template polynucleotide and hybridizing the immobilized complement of the template polynucleotide to a second oligonucleotide, wherein the second oligonucleotide includes a cleavable site and is attached to the solid support, and extending with a polymerase the second oligonucleotide to generate an immobilized copy of the template polynucleotide; c) repeating steps a) and b) one or more times, thereby forming a plurality of immobilized amplification products on the solid support, each amplification product includes the cleavable site; d) contacting a fraction of the cleavable sites with a cleaving agent to remove a fraction of the plurality of amplification products; and e) repeating steps a) and b) one or more times.

In another aspect is provided a method of amplifying a template polynucleotide on a solid support. In embodiments, the method includes (i) executing one or more amplification cycles thereby forming a plurality of immobilized amplification products including a cleavable site on the solid support, wherein each amplification cycle includes: a) hybridizing the template polynucleotide to a first oligonucleotide, wherein the first oligonucleotide includes a cleavable site and is attached to the solid support, and extending the first oligonucleotide with a polymerase to generate an immobilized complement of the template polynucleotide; b) denaturing the template polynucleotide and immobilized complement; c) hybridizing the immobilized complement to a second oligonucleotide, wherein the second oligonucleotide includes a cleavable site and is attached to the solid support, and extending the second oligonucleotide with a polymerase to generate an immobilized copy of the template polynucleotide; and (ii) incubating a fraction of the cleavable sites with a cleaving agent to remove a fraction of the plurality of immobilized amplification products; and (iii) after step (ii), executing one or more amplification cycles. In embodiments, the method does not include sequencing the amplification products prior to step (iii). In embodiments, the method further includes repeating steps (ii) and (iii).

### BRIEF DESCRIPTION OF THE DRAWINGS

FIG. 1 is a graph illustrating the results of a computer simulation to describe how the monoclonal occupancy of wells on a solid support (e.g., a patterned flow cell) can vary depending on initial template hybridization and extension (also referred to herein as “seeding”) conditions (e.g., by fraction of monoclonal sites after one round of seeding, x-axis) and the number of seeding events (2 to 16 events modeled: diamonds: 2 events; squares: 3 events; triangles: 4 events; crosses: 16 events). This method of re-seeding (i.e., performing multiple rounds of template hybridization and extension) is referred to herein as “staircase amplification”.

FIGS. 2A-2C illustrate one embodiment of the invention for generating monoclonal clusters, including the steps of hybridizing templates onto a plurality of immobilized primers on a solid support, extending the primers, amplification to generate clusters, and random removal of the majority of the templates, followed by repeating the amplification and removal steps (also referred to herein as the S-AD method). FIG. 2A illustrates the steps of randomly seeding (i.e., hybridizing templates to immobilized primers and extending the primers, thereby immobilizing the complements of the templates) a diverse population of templates onto a plurality of immobilized primers. Following seeding, cluster amplification is performed to generate a polyclonal cluster containing, for example, 3 different template species. FIG. 2B illustrates the steps of randomly removing the majority of the templates from the polyclonal cluster (e.g., via digestion), followed by more cycles of amplification to regenerate the cluster. In this example, the polyclonal cluster now contains two different species of templates. FIG. 2C illustrates the steps of randomly remove the majority of the templates from the polyclonal cluster (e.g., via digestion), resulting in a single species of template. Another round of amplification results in monoclonal cluster.

FIG. 3 is a graph illustrating the results of a computer simulation to determine the fraction of monoclonal clusters versus the number of cluster amplification and digestion rounds (e.g., 8 rounds) performed for the S-AD method, as described in FIGS. 2A-2C. 1,000 spots were simulated, with each spot containing up to 1000 templates following cluster amplification. During seeding, simulation parameters included a 90% probability that a spot is seeded with at least one template. During the digestion step, simulation parameters included that 99.5% of the templates were digested during each removal step.

FIGS. 4A-4C illustrate one embodiment of the invention for generating monoclonal clusters including repeating the steps of seeding (i.e., hybridizing a template to an immobilized primer and extending the primer to immobilize the complement of the template) a population of templates onto a solid support, randomly removing a majority of the templates, and amplification (also referred to herein as the SDA method). FIG. 4A illustrates the steps of randomly seeding a diverse population of templates onto a plurality of immobilized primers. Following seeding, random removal of the majority of the templates from the polyclonal cluster (e.g., via digestion) is performed, followed by cycles of amplification to amplify the polyclonal cluster. FIG. 4B illustrates the steps of re-seeding a population of templates onto the plurality of immobilized primers, followed by randomly removing the majority of the templates from the polyclonal cluster (e.g., via digestion). In this example, the polyclonal cluster now contains one species of templates. FIG. 4C illustrates the step of performing another round of amplification, resulting in a monoclonal cluster.

FIG. 5 is a graph illustrating the results of a computer simulation comparing of the performance of the SDA (circle: p(digest)=0.95), S-AD (triangle: p(digest)=0.95), and staircase amplification methods using the best performing conditions for each method as determined by the fraction of monoclonal clusters (y-axis) after each round of the clustering process (x-axis). Staircase 1 (star: p(seed)=0.025) corresponds to the seeding condition that produced the highest fraction of monoclonal clusters after 10 rounds of the clustering process, while Staircase 2 (diamond: p(seed)=0.05) corresponds to the seeding condition that produced the highest fraction of monoclonal clusters after 2 rounds of the clustering process. p(seed) represents the probability of seeding a subset of the spots (e.g., 5% probability of seeding a spot), and p(digest) represents the probability of randomly removing a template in a spot (e.g., 95% probability of removing a template).

FIG. 6 is a graph illustrating the results of a computer simulation to comparing the fraction of monoclonal features (y-axis) of the SDA(circle: p(digest)=0.95), S-AD (triangle: p(digest)=0.95), and staircase amplification (diamond) methods after 10 rounds of the process (y-axis) as a function of the relative seeding input (x-axis) (e.g., template immobilization probability).

FIG. 7 is a graph illustrating the results of a computer simulation comparing the fraction of monoclonal features (y-axis) of the SDA(circle: p(digest)=0.95), S-AD (triangle: p(digest)=0.95), and staircase amplification (diamond) methods after 10 rounds of the process as a function of the relative seeding input (x-axis) (e.g., template immobilization probability), with the number of templates per spot increased from 100 to 40,000 templates, and 15 PCR amplification cycles per round in between seeding events. Fraction monoclonal features indicates the fraction of spots in the simulation having >95% template purity.

FIG. 8 illustrates an embodiment of the invention to reduce the number of PCR cycles required per round of clustering while maintaining large clusters (e.g., ˜40k template molecules per spot). A solid support (e.g., a patterned flow cell) is produced, and includes particles (e.g., the particles shown in the center of each square feature) containing a limited number of adapter oligos (e.g., 50, 100, or 1,000 adapter molecules, e.g. supporting a template copy number that is insufficient for sequencing) deposited within larger sub-surfaces (squares above) containing activatable adapter oligos. The sub-surfaces are separated by non-templatable interstitial space, which refers to space that cannot harbor any template molecules. Clustering according to the methods described herein is performed on the surface of the particles, enabling the production of monoclonal particles with a minimal number of PCR cycles. Following clustering, the activatable surface is rendered active (e.g., by cleavage/digestion of a blocking moiety) and the monoclonal templates on the particles are copied onto the activatable surface, thereby producing large monoclonal colonies. Cluster separation improves signal deconvolution and minimizes spreading linked to ‘optical’ duplicates.

FIGS. 9A-9B illustrates competition between different templates for clonal dominance on individual spots following bottleneck clustering. FIG. 9A shows three spots, A, B, and C, wherein spot A contains two immobilized templates (T1 and T2), spot B contains three immobilized templates (T1, T2, and T3), and spot C includes two immobilized templates (T1 and T3). Following, for example, one round of a bottleneck clustering process as described herein, the quantity of T1 template has grown in spot A, while T1 template in spot B is removed, e.g., during digestion, allowing T2 template to increase in quantity. T3 template in spot C has also increased in quantity, with T1 template also being lost in spot C (i.e., forming a monoclonal T3 cluster in spot C). FIG. 9B illustrates the generation of monoclonal clusters following N rounds of bottleneck clustering. For example, spot A has only T1 template, spot B has only T2 template, and spot C continues to have only T3 template. With subsequent rounds of clustering, T3 template in spot C continues to grow in density.

FIGS. 10A-10B show a series of histograms comparing the purity per cluster (i.e., the fraction of cluster templates including the most abundant cluster species) over several rounds of bottleneck clustering. FIG. 10A illustrates the purity per cluster following 0, 1, 2, and 3 rounds of bottleneck clustering. FIG. 10B illustrates the purity per cluster following 4, 5, 6, and 7 rounds of bottleneck clustering.

FIG. 11 is an illustration of one embodiment of a library molecule for use in the invention. The library adapters may include pp1/pp2 sequences (i.e., platform primer binding sequences, for example, P5/P7 or S1/S2), and a sequencing primer binding sequence. The adapters may each include restriction endonuclease recognition sites (RE sites), for example BglII or NotI.

FIG. 12 illustrates an exemplary strategy for optimizing digestion efficiency of clusters on a solid support. A patterned flow cell, for example, is contacted with a predetermined number of template molecules to produce a plurality of spots seeded with a single template molecule. Following seeding, the single template molecules are amplified by solid phase amplification methods (e.g., chemical bridge amplification) to generate clusters, and then the number of clusters is quantified (e.g., by fluorescence microscopy using a labeled oligonucleotide probe complementary to a sequence on an amplicon in each cluster) to obtain the baseline number of clusters. The process is repeated, this time with the addition of a cleavage step (e.g., via a restriction endonuclease) prior to clustering and quantification. The number of clusters measured following digestion is compared to the baseline number of clusters to determine the digestion efficiency (e.g., the cleavage efficiency is equal to the number of clusters measured following digestion divided by the baseline number of clusters).

FIG. 13 illustrates an embodiment of a programmable endonuclease-based approach for generating monoclonal clusters. A programmable endonuclease, for example, a Thermus thermophilus argonaut (TtAgo) enzyme and an associated guide oligo, with a length of between 16 to 18 nucleotides, are used to target immobilized templates. TtAgo cleaves a complementary polynucleotide between the bases corresponding to positions 10 and 11 of the DNA guide oligo. With respect to an immobilized template sequence including a platform primer sequence (e.g., S1 or S2, or a complement thereof) and a sequencing primer binding sequence (e.g., SP1 or SP2, or a complement thereof), a TtAgo guide oligo is designed, for example, such that the first 10 nucleotide on the 5′ end of the guide oligo are complementary to the sequencing primer binding sequence, and the adjacent, downstream 6 to 8 nucleotides are complementary to the platform primer sequence, or complement thereof. Addition of a TtAgo and guide oligo complex result in targeting of the TtAgo complex to the immobilized template. Note, that while only one immobilized template is illustrated, it is to be understood that a plurality of immobilized templates would be present on the solid support. The dotted lines indicate additional template sequence that is not illustrated in the figure.

FIG. 14 illustrates additional steps of an embodiment of a programmable endonuclease-based approach for generating monoclonal clusters. A TtAgo and guide oligo complex (i.e., a TtAgo complex) specific for an immobilized template, as described in FIG. 13, is added to a solid support including a plurality of immobilized templates. In embodiments, more than one plurality of TtAgo complexes is added to the support, wherein the guide oligo of each plurality is complementary to a specific platform primer sequence and sequencing primer binding sequence combination. For example, 5 different pluralities of TtAgo complexes are added to a solid support including, for example, immobilized template polynucleotides including one of 6 different platform primer sequences, such that 1 out of the 6 platform primer sequences are targeted for cleavage by each TtAgo complex. As illustrated in FIG. 14, a TtAgo complex contacts a solid support including a first immobilized template polynucleotide including, from 5′ to 3′, a first platform primer sequence (e.g., S1), a first sequencing primer binding sequence (e.g., SP1), an insert (e.g., insert 2), a first sequencing primer binding sequence complement (e.g., SP2′) and a first platform primer sequence complement (e.g., S2′), and a second immobilized template polynucleotide including, from 5′ to 3′, a second platform primer sequence (e.g., S2), a second sequencing primer binding sequence (e.g., SP2), an insert (e.g., insert 1), a second sequencing primer binding sequence complement (e.g., SP1′), and a second platform primer sequence complement (e.g., S1′). The complexed guide oligo is complementary to a portion of the second platform primer sequence and the second sequencing primer binding sequence, following a sequence complementarity scheme as described in FIG. 13. Following an incubation with the TtAgo complex, the second immobilized template polynucleotide is cleaved between the 10^{th} and 11^{th} nucleotides from the 5′ end of the second platform primer sequence, releasing the second template polynucleotide such that the solid support now includes a free second platform primer sequence (e.g., a second platform primer sequence with an extendable 3′ end) that may participate in additional amplification cycles with other immobilized template polynucleotide including a complementary platform primer binding sequence.

FIGS. 15A-15C illustrates an embodiment for generating monoclonal clusters using a programmable-based approach, including the steps of hybridizing templates onto a plurality of immobilized primers on a solid support, extending the primers, amplification to generate clusters, and selective removal of the majority of the templates, followed by repeating the amplification step (and optionally, the removal step). FIG. 15A illustrates the steps of randomly seeding (i.e., hybridizing templates to immobilized primers and extending the primers, thereby immobilizing the complements of the templates) a diverse population of templates onto a plurality of immobilized platform primers (e.g., 6 pluralities of immobilized platform primers, or between 4 to 12 or more pluralities of immobilized platform primers). Following seeding, cluster amplification is performed to generate a polyclonal cluster containing, for example, 4 different immobilized template species. FIG. 15B illustrates the steps of selective targeting the majority of the templates from the polyclonal cluster with a TtAgo complex (e.g., A TtAgo enzyme and guide oligo complex, wherein the guide oligo is specific for the platform primer sequence and sequencing primer binding sequence of one or more immobilized template polynucleotides, as described in FIGS. 13-14). Following targeting of a subset of the platform primer sequences (e.g., 5 out of 6 platform primer sequences), the TtAgo-bound templates are cleaved and removed, as shown in FIG. 15C. Additional rounds of amplification result in a monoclonal cluster.

### DETAILED DESCRIPTION

The aspects and embodiments described herein relate to methods and compositions for increasing polynucleotide template clustering and amplification efficiency on solid support.

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. When referring to a double-stranded polynucleotide including a first strand hybridized to a second strand, it is to be understood that each of the terms “first strand” and “second strand” refer to single-stranded polynucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, nucleic acid, a protein, or enzyme (e.g., a DNA polymerase).

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis. The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support (e.g., a polymer coated solid support). In embodiments, forward primers anneal to the antisense strand of the double-stranded DNA, which runs from the 3′ to 5′ direction. Forward primers, for example, initiate the synthesis of a gene in the 5′ to 3′ direction. In embodiments, reverse primers anneal to the sense strand of the double-stranded DNA, which runs from the 5′ to 3′ direction. Reverse primers, for example, initiate the synthesis of a gene in the 3′ to 5′ direction. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, a “template complex” refers to a double stranded nucleic acid complex formed as a result of a hybridization event between a DNA template molecule and a primer. In embodiments, the formation of a template complex enables elongation at the 3′ end of the primer. In embodiments, the primer is an oligonucleotide that includes a cleavable site and is immobilized to a solid support. In embodiments, the template complex is contacted with a polymerase capable of extending the immobilized oligonucleotide to form a plurality of extended complements of the templates.

As used herein, the term “random” in the context of a nucleic acid sequence or barcode sequence refers to a sequence where one or more nucleotides has an equal probability of being present. In embodiments, one or more nucleotides is selected at random from a set of two or more different nucleotides at one or more positions, with each of the different nucleotides selected at one or more positions represented in a pool of oligonucleotides including the random sequence. For example, a random sequence may be represented by a sequence composed of N's, where N can be any nucleotide (e.g., A, T, C, or G). For example, a four base random sequence may have the sequence NNNN, where the Ns can independently be any nucleotide (e.g., AATC). Interposing oligonucleotide probes that contain a random sequence, collectively, have sequences composed of Ns within the hybridization sequences, stem region, or loop region.

As used herein, the terms “solid support” and “substrate” and “solid surface” are used interchangeably and refers to discrete solid or semi-solid surfaces to which a plurality of nucleic acid (e.g., primers) may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may include a discrete particle that may be spherical (e.g., microspheres) or have a non-spherical or irregular shape, such as cubic, cuboid, pyramidal, cylindrical, conical, oblong, or disc-shaped, and the like. Solid supports may be in the form of discrete particles, which alone does not imply or require any particular shape. The term “particle” means a small body made of a rigid or semi-rigid material. The body can have a shape characterized, for example, as a sphere, oval, microsphere, or other recognized particle shape whether having regular or irregular dimensions. As used herein, the term “discrete particles” refers to physically distinct particles having discernible boundaries. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. Discrete particles collected in a container and contacting one another will define a bulk volume containing the particles, and will typically leave some internal fraction of that bulk volume unoccupied by the particles, even when packed closely together. In embodiments, cores and/or core-shell particles are approximately spherical. As used herein the term “spherical” refers to structures, which appear substantially, or generally of spherical shape to the human eye, and does not require a sphere to a mathematical standard. In other words, “spherical” cores or particles are generally spheroidal in the sense of resembling or approximating to a sphere. In embodiments, the diameter of a spherical core or particle is substantially uniform, e.g., about the same at any point, but may contain imperfections, such as deviations of up to 1, 2, 3, 4, 5 or up to 10%. Because cores or particles may deviate from a perfect sphere, the term “diameter” refers to the longest dimension of a given core or particle. Likewise, polymer shells are not necessarily of perfect uniform thickness all around a given core. Thus, the term “thickness” in relation to a polymer structure (e.g., a shell polymer of a core-shell particle) refers to the average thickness of the polymer layer.

A solid support may further include a polymer or hydrogel on the surface to which the primers are attached (e.g., the primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface comprising a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip, surface of a particle), for example a metal surface (e.g., steel, gold, silver, aluminum, silicon and copper). In some embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, silica, nylon, Wang resin, Merrifield resin, metal (e.g., iron, a metal alloy, sepharose, agarose, polyacrylamide, dextran, cellulose and the like or combinations thereof. In some embodiments a substrate includes a magnetic material (e.g., iron, nickel, cobalt, platinum, aluminum, and the like). In certain embodiments a substrate includes a magnetic bead (e.g., DYNABEADS®, hematite, AMPure XP). Magnets can be used to purify and/or capture nucleic acids bound to certain substrates (e.g., substrates comprising a metal or magnetic material).

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer. Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

The term “surface” is intended to mean an external part or external layer of a substrate. The surface can be in contact with another material such as a gas, liquid, gel, polymer, organic polymer, second surface of a similar or different material, metal, or coating. The surface, or regions thereof, can be substantially flat. The substrate and/or the surface can have surface features such as wells, pits, channels, ridges, raised regions, pegs, posts or the like.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

As used herein, the term “template polynucleotide” or “template nucleic acid” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The terms “single strand” and “ssDNA” are used in accordance with its plain and ordinary meaning and refer to a single-stranded polynucleotide. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In the context of selective sequencing, “target polynucleotide(s)” refers to the subset of polynucleotide(s) to be sequenced from within a starting population of polynucleotides.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g. apoptosis or necrosis) or cell shedding, releasing polynucleotides into surrounding body fluids or into circulation. Accordingly, cell-free polynucleotides may be isolated from a non-cellular fraction of blood (e.g. serum or plasma), from other bodily fluids (e.g. urine), or from non-cellular fractions of other types of samples.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog (e.g., a reversible terminating moiety). Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate). A “canonical” nucleotide is an unmodified nucleotide.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety (alternatively referred to herein as a reversible terminator moiety) and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

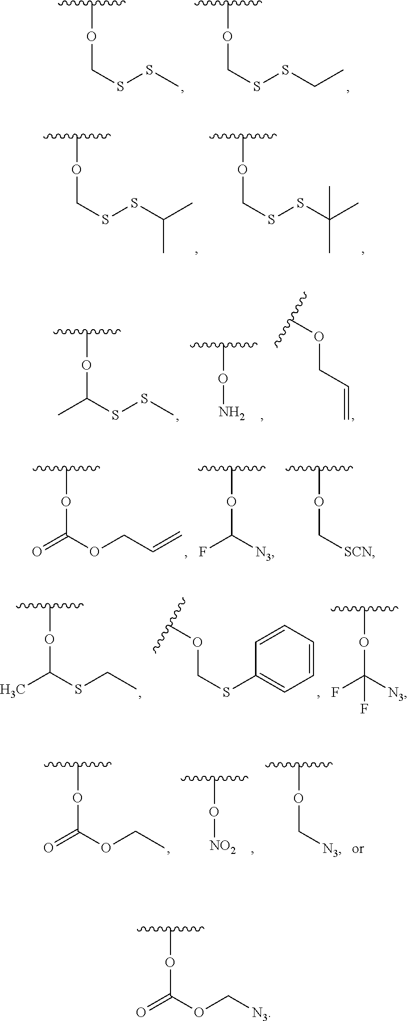

A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. As used herein, a “cleaving agent” is an agent capable of cleaving a cleavable linker and/or a cleavable moiety. Examples of a cleaving agent includes a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, the immobilized oligonucleotide as described herein includes a cleavable site, and cleaving at or near the cleavable site on the immobilized oligonucleotide includes removing the immobilized oligonucleotide. In embodiments, cleaving includes removing a fraction of a plurality of immobilized amplification products generated using the method described herein. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

As used herein, the term “loop” is used in accordance with its plain ordinary meaning and refers to the single-stranded region of a hairpin adapter that is located between the duplexed “stem” region of the hairpin adapter. In embodiments, the hairpin loop region is between about 4 nucleotides to 150 nucleotides in length. In embodiments, the hairpin loop is at least 4, 5, 6, 7, 8, 9, 10, 15, 20, 25, 30, 35, 40, 45, 50, or more nucleotides in length. In embodiments, the hairpin loop includes 1, 2, 3, 4, 5, 6, 7, 8, 9, 10, or more T nucleotides. In embodiments, the hairpin loop may include one or more of a primer binding sequence, a barcode, a UMI sequence, or a cleavable site. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site blast.ncbi.nlm.nih.gov/Blast.cgi or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. Additional examples of a reversible terminator may be found in U.S. Pat. Nos. 6,664,079; 6,214,987; 5,872,244; Ju J. et al. (2006) Proc Natl Acad Sci USA 103(52):19635-19640; Ruparel H. et al. (2005) Proc Natl Acad Sci USA 102(17):5932-5937; Wu J. et al. (2007) Proc Natl Acad Sci USA 104(104):16462-16467; Guo J. et al. (2008) Proc Natl Acad Sci USA 105(27): 9145-9150 Bentley D. R. et al. (2008) Nature 456(7218):53-59; or Hutter D. et al. (2010) Nucleosides Nucleotides & Nucleic Acids 29:879-895, which are incorporated herein by reference in their entirety for all purposes. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is

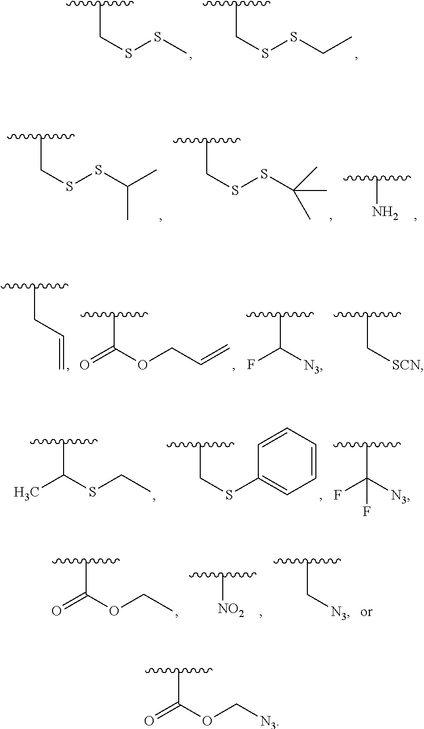

The term “allyl” as described herein refers to an unsubstituted methylene attached to a vinyl group (i.e., —CH═CH₂), having the formula

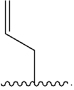 [SMILES: *CC=C]

In embodiments, the reversible terminator moiety is

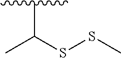 [SMILES: *C(C)SSC]

as described in U.S. Pat. No. 10,738,072, which is incorporated herein by reference for all purposes. For example, a nucleotide including a reversible terminator moiety may be represented by the formula:

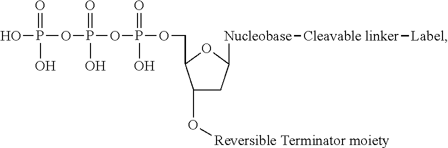

where the nucleobase is adenine or adenine analogue, thymine or thymine analogue, guanine or guanine analogue, or cytosine or cytosine analogue. In embodiments, the reversible terminator includes a hydrocarbyl. In embodiments, the reversible terminator includes an ester (O—C(O)R₁′ wherein R₁′ is any alkyl or aryl group which can include a formate, benzoyl formate, acetate, substituted acetate, propionate, and other esters as described in Green, T. W. (Protective Groups in Organic Chemistry, Wiley & Sons, New York, 1981)). In embodiments, the reversible terminator includes an ether (O—R₂′ wherein R₂′ can be substituted or unsubstituted alkyl such as methyl, substituted methyl, ethyl, substituted ethyl, allyl, substituted benzyl, silyl, or any other ether used to transiently protect hydroxyls and similar groups). In embodiments, the reversible terminator includes an O—CH₂(OC₂H₅)_{N′} CH₃ wherein N′ is an integer from 1-10. In embodiments, the reversible terminator includes a phosphate, phosphoramidate, phosphoramide, toluic acid ester, benzoic ester, acetic acid ester, or ethoxyethyl ether.

In some embodiments, a nucleic acid includes a molecular identifier or a molecular barcode. As used herein, the term “molecular barcode” (which may be referred to as a “tag”, a “barcode”, a “molecular identifier”, an “identifier sequence”, an “index”, or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads comprising the barcode can be identified as originating from a single sample polynucleotide molecule on the basis of the barcode alone. In other embodiments, individual barcode sequences may be used more than once, but adapters comprising the duplicate barcodes are associated with different sequences and/or in different combinations of barcoded adapters, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random.

As used herein, the terms “denaturant” or plural “denaturants” are used in accordance with their plain and ordinary meanings and refer to an additive or condition that disrupts the base pairing between nucleotides within opposing strands of a double-stranded polynucleotide molecule. The term “denature” and its variants, when used in reference to any double-stranded polynucleotide molecule, or double-stranded polynucleotide sequence, includes any process whereby the base pairing between nucleotides within opposing strands of the double-stranded molecule, or double-stranded sequence, is disrupted. In embodiments, denaturing refers to denaturing a template polynucleotide and the immobilized complement. Typically, denaturation includes rendering at least some portion or region of two strands of the double-stranded polynucleotide molecule or sequence single-stranded or partially single-stranded. In some embodiments, denaturation includes separation of at least some portion or region of two strands of the double-stranded polynucleotide molecule or sequence from each other. Typically, the denatured region or portion is then capable of hybridizing to another polynucleotide molecule or sequence. Optionally, there can be “complete” or “total” denaturation of a double-stranded polynucleotide molecule or sequence. Complete denaturation conditions are, for example, conditions that would result in complete separation of a significant fraction (e.g., more than 10%, 20%, 30%, 40% or 50%) of a large plurality of strands from their extended and/or full-length complements. Typically, complete or total denaturation disrupts all of the base pairing between the nucleotides of the two strands with each other. Similarly, a nucleic acid sample is optionally considered fully denatured when more than 80% or 90% of individual molecules of the sample lack any double-strandedness (or lack any hybridization to a complementary strand).

Optionally, a nucleic acid sample can be considered to be partially denatured when a substantial fraction of individual nucleic acid molecules of the sample (e.g., above 20%, 30%, 50%, or 70%) are in a partially denatured state. Optionally less than a substantial amount of individual nucleic acid molecules in the sample are fully denatured, e.g., not more than 5%, 10%, 20%, 30% or 50% of the nucleic acid molecules in the sample. Under exemplary conditions at least 50% of the nucleic acid molecules of the sample are partly denatured, but less than 20% or 10% are fully denatured. In other situations, at least 30% of the nucleic acid molecules of the sample are partly denatured, but less than 10% or 5% are fully denatured. Similarly, a nucleic acid sample can be non-denatured when a minority of individual nucleic acid molecules in the sample are partially or completely denatured.

In an embodiment, partially denaturing conditions are achieved by maintaining the duplexes as a suitable temperature range. For example, the nucleic acid is maintained at temperature sufficiently elevated to achieve some heat-denaturation (e.g., above 45° C., 50° C., 55° C., 60° C., 65° C., or 70° C.) but not high enough to achieve complete heat-denaturation (e.g., below 95° C. or 90° C. or 85° C. or 80° C. or 75° C.). In an embodiment the nucleic acid is partially denatured using substantially isothermal conditions. Alternatively, chemical denaturation can be accomplished by contacting the double-stranded polynucleotide to be denatured with appropriate chemical denaturants, such as strong alkalis, strong acids, chaotropic agents, and the like and can include, for example, NaOH, urea, or guanidine-containing compounds. In some embodiments, partial or complete denaturation is achieved by exposure to chemical denaturants such as urea or formamide, with concentrations suitably adjusted, or using high or low pH (e.g., pH between 4-6 or 8-9). In embodiments, the denaturant is a buffered solution including betaine, dimethyl sulfoxide (DMSO), ethylene glycol, formamide, glycerol, guanidine thiocyanate, 4-methylmorpholine 4-oxide (NMO), or a mixture thereof. In embodiments, the first denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof. In an embodiment herein, partial denaturation and/or amplification, including any one or more steps or methods described herein, can be achieved using a recombinase and/or single-stranded binding protein.

In some embodiments, complete or partial denaturation of a double-stranded polynucleotide sequence is accomplished by contacting the double-stranded polynucleotide sequence using appropriate denaturing agents. For example, the double-stranded polynucleotide can be subjected to heat-denaturation (also referred to interchangeably as thermal denaturation) by raising the temperature to a point where the desired level of denaturation is accomplished. In some embodiments, thermal denaturation of a double-stranded polynucleotide, includes adjusting the temperature to achieve complete separation of the two strands of the polynucleotide, such that 90% or greater of the strands are in single-stranded form across their entire length. A completely denatured double-stranded polynucleotide results in a separated first strand and a second strand, each of which is a single-stranded polynucleotide. In some embodiments, complete thermal denaturation of a polynucleotide molecule (or polynucleotide sequence) is accomplished by exposing the polynucleotide molecule (or sequence) to a temperature that is at least 5° C., 10° C., 15° C., 20° C., 25° C., 30° C., 50° C., or 100° C., above the calculated or predict melting temperature (Tm) of the polynucleotide molecule or sequence.

In some embodiments, complete or partial denaturation is accomplished by treating the double-stranded polynucleotide sequence to be denatured using a denaturant mixture including an SSB protein (e.g., T4 gp32 protein, T7 gene 2.5 SSB protein, or phi29 SSB protein, Thermococcus kodakarensis (KOD) SSB, Thermus thermophilus (TTH) SSB, Sulfolobus solfataricus (SSO) SSB, or Extreme Thermostable Single-Stranded DNA Binding Protein (ET-SSB)), a strand-displacing polymerase (e.g., Bst large fragment (Bst LF) polymerase, Bst 3.0 polymerase, Bst 2.0 polymerase, Bsu polymerase, SD polymerase, Vent exo-polymerase, Phi29 polymerase, or a mutant thereof), and one or more crowding agents (poly(ethylene glycol) (PEG), polyvinylpyrrolidone (PVP), bovine serum albumin (BSA), dextran, Ficoll (e.g., Ficoll 70 or Ficoll 400), glycerol, or a combination thereof). In embodiments, the crowding agent is poly(ethylene glycol) (e.g., PEG 200, PEG 600, PEG 800, PEG 2,050, PEG 4,600, PEG 6,000, PEG 8,000, PEG 10,000, PEG 20,000, or PEG 35,000), dextran sulfate, bovine pancreatic trypsin inhibitor (BPTI), ribonuclease A, lysozyme, β-lactoglobulin, hemoglobin, bovine serum albumin (BSA), or poly(sodium 4-styrene sulfonate) (PSS). In embodiments, the denaturant mixture including an SSB, a strand-displacing polymerase, and one or more crowding agents does not include a chemical denaturant (e.g., betaine, DMSO, ethylene glycol, formamide, guanidine thiocyanate, NMO, TMAC, or a mixture thereof).

In some embodiments, a nucleic acid includes a label. As used herein, the term “label” or “labels” are used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing).

In embodiments, the detectable label is a fluorescent dye. In embodiments, the detectable label is a fluorescent dye capable of exchanging energy with another fluorescent dye (e.g., fluorescence resonance energy transfer (FRET) chromophores). Examples of detectable agents include imaging agents, including fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a moiety of a derivative of one of the detectable moieties described immediately above, wherein the derivative differs from one of the detectable moieties immediately above by a modification resulting from the conjugation of the detectable moiety to a compound described herein.

The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′→3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “programmable endonuclease,” or “programmable endonuclease complex” refers to different classes of enzymes that can be targeted to cleave a specific region of a DNA or RNA molecule. Thus, a programmable endonuclease is an endonuclease that can be designed or programmed to cleave a nucleotide sequence of interest. For example, a programmable endonuclease can include of target recognition portion and endonuclease portion, where a common endonuclease portion can be combined with any target recognition portion to cleave a nucleotide sequence of interest. In one embodiment, the programmable endonucleases are targeted by a guide RNA (gRNA), a guide DNA (gDNA) or by a structure formed between a guide molecule (e.g., a guide oligonucleotide) and the target. Guide oligonucleotides, such as “guide RNA” refer to a short synthetic oligonucleotides composed of a “scaffold” sequence necessary for endonuclease binding and a user-defined “targeting sequence which defines the target to be modified. For example, Cas9 are programmable endonucleases, as they cleave double stranded genetic material by making a double stranded break at a specific location at a recognition site. Additional examples of programmable endonucleases include Cpf1, C2c1, C2c2, C2c, RNA- or DNA-guided Argonaute proteins, structure-guided endonucleases, among others.

As used herein, the term “incorporating” or “chemically incorporating,” when used in reference to a primer and a nucleotide, refers to the process of joining the nucleotide to the primer or extension product thereof by formation of a phosphodiester bond.

As used herein, the term “selective” or “selectivity” or the like of a compound refers to the compound's ability to discriminate between molecular targets. When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the terms “specific”, “specifically”, “specificity”, or the like of a compound refers to the agent's ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers comprising tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

As used herein, the terms “sequencing”, “sequence determination”, “determining a nucleotide sequence”, and the like include determination of a partial or complete sequence information, including the identification, ordering, or locations of the nucleotides that include the polynucleotide being sequenced, and inclusive of the physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. An “extension strand” is formed as the one or more nucleotides are incorporated into a complementary polynucleotide hybridized to a template nucleic acid. The extension strand is complementary to the template nucleic acid. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “sequencing reaction mixture” is used in accordance with its plain and ordinary meaning and refers to an aqueous mixture that contains the reagents sufficient to allow a dNTP or dNTP analogue to add a nucleotide to a DNA strand by a DNA polymerase. In embodiments, the sequencing reaction mixture includes a buffer. In embodiments, the buffer includes an acetate buffer, 3-(N-morpholino) propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), or salts (e.g., ammonium sulfate, magnesium chloride, sodium chloride, or potassium chloride).

As used herein, the term “extension” or “elongation” is used in accordance with their plain and ordinary meanings and refer to synthesis by a polymerase of a new polynucleotide strand (i.e., an “extension strand”) complementary to a template strand by adding free nucleotides (e.g., dNTPs) from a reaction mixture that are complementary to the template in a 5′-to-3′ direction, including condensing a 5′-phosphate group of a dNTPs with a 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. The sequence reads are optionally stored in an appropriate data structure for further evaluation. In embodiments, a first sequencing reaction can generate a first sequencing read. The first sequencing read can provide the sequence of a first region of the polynucleotide fragment. In embodiments, a second sequencing primer can initiate sequencing at a second location (e.g., a sequencing primer binding sequence) on the nucleic acid template. The second location can be distinct from the first location. In some cases, a 3′ terminal nucleotide of the second primer can hybridize to a location that is more than 5 nucleotides away from a binding site of a 3′ terminal nucleotide of the first primer. The second sequencing reaction can generate a second sequencing read. The second sequencing read can provide the sequence of a second region of the nucleic acid template which is distinct from the first region of the nucleic acid template. In some embodiments, the nucleic acid template is optionally subjected to one or more additional rounds of sequencing using additional sequencing primers, thereby generating additional sequencing reads.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

As used herein, the term “hybridize” or “specifically hybridize” refers to a process where two complementary nucleic acid strands anneal to each other under appropriately stringent conditions. Hybridizations are typically and preferably conducted with oligonucleotides. The terms “annealing” and “hybridization” are used interchangeably to mean the formation of a stable duplex. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. Those skilled in the art understand how to estimate and adjust the stringency of hybridization conditions such that sequences having at least a desired level of complementarity will stably hybridize, while those having lower complementarity will not. As used herein, the term “stringent condition” refers to condition(s) under which a polynucleotide probe or primer will hybridize preferentially to its target sequence, and to a lesser extent to, or not at all to, other sequences. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands (e.g., two single-stranded polynucleotides) that are hybridized to each other can form a duplex which includes a double-stranded portion of nucleic acid.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction includes a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

A nucleic acid can be amplified by a thermocycling method or by an isothermal amplification method. In some embodiments, a rolling circle amplification method is used. In some embodiments, amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction comprising only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., US patent publication US 2013/0012399), the like or combinations thereof.

In embodiments, amplification includes executing one or more amplification cycles. In embodiments, the first oligonucleotide includes a cleavable site and is attached to the solid support, and extending the first oligonucleotide with a polymerase generates an immobilized complement of the template polynucleotide. In embodiments, denaturing includes denaturing the template polynucleotide and immobilized complement. In embodiments, the amplification cycle includes hybridizing the immobilized complement to a second oligonucleotide, wherein the second oligonucleotide includes a cleavable site and is attached to the solid support, and extending the second oligonucleotide with a polymerase generates an immobilized copy of the template polynucleotide. In embodiments, executing one or more amplification cycles forms a plurality of immobilized amplification products comprising a cleavable site on the solid support.

As used herein, the term “sparse-seed cycle” refers to a process as described herein for attaching template polynucleotides to a solid support, followed by removing one or more template polynucleotides, of the complement thereof. In embodiments, a sparse-seed cycle includes contacting a solid support with a plurality of template polynucleotides and forming a plurality of template complexes, wherein each template complex includes a template polynucleotide hybridized to an immobilized oligonucleotide with a cleavable site; contacting the template complexes with a polymerase and extending the immobilized oligonucleotide to form a plurality of extended complements of templates; and removing a fraction (e.g., greater than 80%) of the extended complements of templates.

Provided herein are methods and compositions for analyzing a sample (e.g., sequencing nucleic acids within a sample). A sample (e.g., a sample comprising nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes nucleic acid, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system comprising two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate | Bioconjugate | |
|---|---|---|
| reactive | reactive | |
| group 1 | group 2 | Resulting |
| (e.g., electrophilic | (e.g., nucleophilic | Bioconjugate |
| bioconjugate | bioconjugate | reactive |
| reactive moiety) | reactive moiety) | linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or strepavidin to form a avidin-biotin complex or streptavidin-biotin complex.

The term “covalent linker” is used in accordance with its ordinary meaning and refers to a divalent moiety, which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

The term “adapter” as used herein refers to any linear oligonucleotide that can be ligated to a nucleic acid molecule, thereby generating nucleic acid products that can be sequenced on a sequencing platform (e.g., an Illumina or Singular Genomics G4™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences (e.g., sequencing primer binding sequences) that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ system), Illumina™ (e.g., HiSeq™ MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Methods

In an aspect is provided a method of amplifying a template polynucleotide on a solid support. In embodiments, the method includes a) hybridizing the template polynucleotide to a first oligonucleotide, wherein the first oligonucleotide comprises a cleavable site and is attached to the solid support, and extending with a polymerase the first oligonucleotide to generate an immobilized complement of the template polynucleotide; b) denaturing the template polynucleotide; c) contacting a fraction of the cleavable sites with a cleaving agent to remove a fraction of the plurality of immobilized amplification products; d) repeating step a); e) denaturing the template polynucleotide and hybridizing the immobilized complement of the template polynucleotide to a second oligonucleotide, wherein the second oligonucleotide comprises a cleavable site and is attached to the solid support, and extending with a polymerase the second oligonucleotide to generate an immobilized copy of the template; f) repeating steps d) and e) one or more times, thereby forming a plurality of immobilized amplification products on the solid support, each amplification product comprises a cleavable site. In embodiments, the method further includes repeating step c) one or more times. In embodiments, the method further includes repeating steps a)-f) one or more times. In embodiments, the method further includes repeating steps d)-f) one or more times. Repeating steps, such as step c), where step c includes contacting a fraction of the cleavable sites with a cleaving agent, 1 or more times means that the number of steps to be performed is one, and the number of steps is increased.

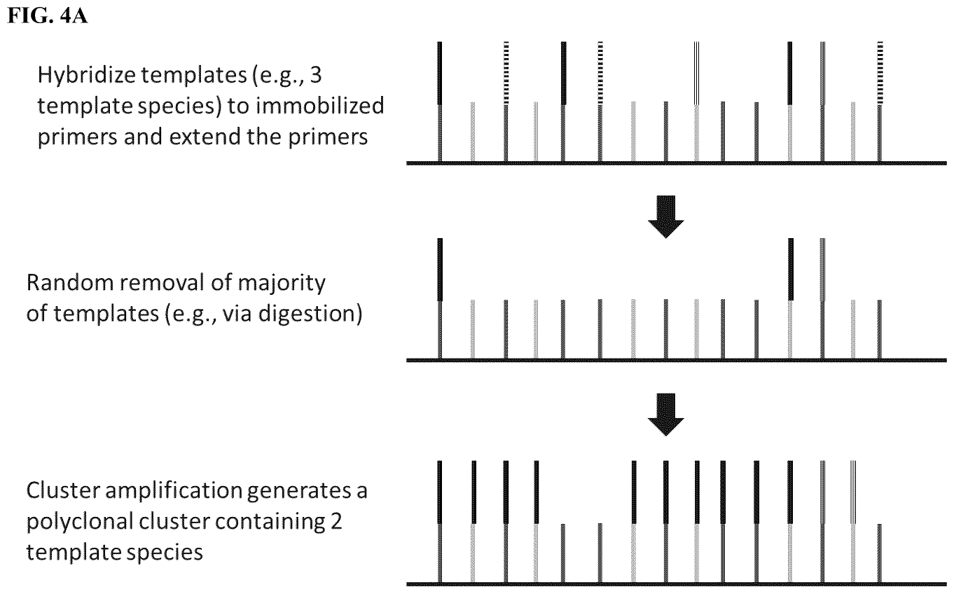

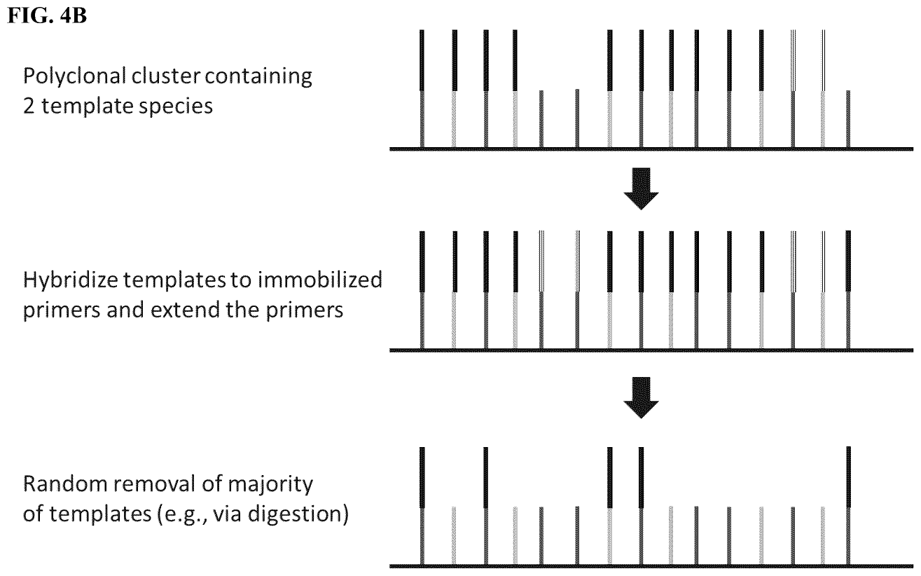

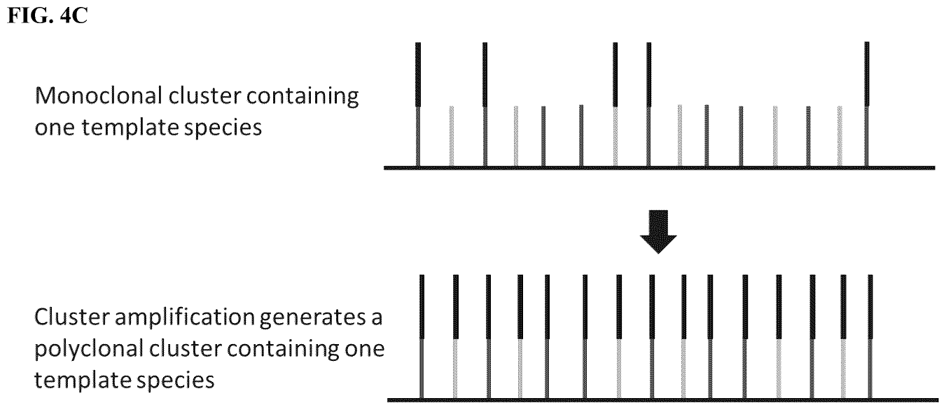

In embodiments, the method further includes repeating step c) (i.e., contacting a fraction of the cleavable sites with a cleaving agent to remove a fraction of the plurality of amplification products). In embodiments, the method further includes repeating step c) one or more times. In embodiments, the method further includes repeating step c) 1, 2, 3, 4, 5, 6, 7, 8, 9, 10, or more times. In embodiments, the method further includes repeating step c) 10 or more times. In embodiments, the method further includes repeating step c) 15, 20, 25, 30, 35, or more times. Repeating steps (e.g., step c)) 1 or more times means that the number of steps to be performed is one, and the number of steps is increased. In embodiments, repeating step c) 2 times means contacting a fraction of the cleavable sites for the first time with a cleaving agent to remove a fraction of the plurality of immobilized amplification products, followed by the hybridization of the template polynucleotide with a cleavable site to an oligonucleotide immobilized onto the solid support to generate an immobilized complement of the template polynucleotide, removal the template polynucleotide, hybridization of the immobilized complement of the template polynucleotide to a second immobilized oligonucleotide to facilitate generating a plurality of immobilized amplification products with cleavable sites, and contacting the fraction of the cleavable sites for a second time with a cleaving agent to remove a fraction of the plurality of immobilized amplification products. FIG. 4A depicts contacting a fraction of the cleavable sites for the first time with a cleaving agent to remove a fraction of the plurality of immobilized amplification products, and FIG. 4B illustrates contacting a fraction of the cleavable sites on the same solid support for the second time with a cleaving agent to remove a fraction of the plurality of immobilized amplification products.

In embodiments, step c) is repeated 1 to about 10 times in total. In embodiments, step b) is repeated between 10 to about 15 times. In embodiments, step c) is repeated 1, 2, 3, 4, 5, 6, 7, 8, 9, or 10 times. In embodiments, step c) is repeated 10, 11, 12, 13, 14, or 15 times. In embodiments, step c) is repeated 5 or more times. In embodiments, step c) is repeated 10 or more times. In embodiments, step c) is repeated more than 15 times.

In embodiments, step c) further includes incubating the fraction of cleavable sites with the cleaving agent for between about 5 seconds up to about 30 minutes. In embodiments, step c) further includes incubating the fraction of cleavable sites with the cleaving agent for about 5 seconds to about 30 seconds, about 30 seconds to about 60 seconds, about 1 minute to about 5 minutes, about 5 minutes to about 15 minutes, or about 15 minutes to about 30 minutes. In embodiments, step c) further includes incubating the fraction of cleavable sites with the cleaving agent for about 5 seconds, about 10 seconds, about 20 seconds, about 30 seconds, about 1 minutes, about 2 minutes, about 3 minutes, about 4 minutes, about 5 minutes, about 10 minutes, about 15 minutes, about 20 minutes, about 25 minutes, about 30 minutes, about 45 minutes, or about 60 minutes. In embodiments, step c) further includes incubating the fraction of cleavable sites with the cleaving agent for between about 1 hour to 2 hours, between about 2 hours to 4 hours, between about 4 hours to 8 hours, between about 8 hours to 12 hours, between about 12 hours to 16 hours, or between about 16 hours to 24 hours. In embodiments, step c) further includes incubating the fraction of cleavable sites with the cleaving agent for between about 1, 2, 3, 4, 5, 6, 7, 8, 9, 10, 11, 12, 13, 14, 15, 16, 17, 18, 19, 20, 21, 22, 23, or 24 hours. In embodiments, step c) further includes incubating the fraction of cleavable sites with the cleaving agent for more than 24 hours.

In embodiments, step c) includes contacting less than 100% of the cleavable sites with a cleaving agent. In embodiments, step c) includes contacting about 90%, about 91%, about 92%, about 93%, about 94%, about 95%, about 96%, about 97%, about 98%, or about 99% of the cleavable sites with the cleaving agent. In embodiments, step c) includes contacting about 95%, about 96%, about 97%, about 98%, or about 99% of the cleavable sites with the cleaving agent. In embodiments, step c) includes contacting about 99% of the cleavable sites with the cleaving agent. In embodiments, step c) includes contacting about 99%, about 99.1%, about 99.2%, about 99.3%, about 99.4% or about 99.5% of the cleavable sites with the cleaving agent. In embodiments, step c) includes contacting about 80% of the cleavable sites with the cleaving agent. In embodiments, step c) includes contacting 80%, 85%, 90% or more of the cleavable sites with the cleaving agent. In embodiments, step c) includes contacting greater than 90% of the cleavable sites with the cleaving agent.

In embodiments, step c) includes contacting a first fraction of the cleavable sites with a first plurality of TtAgo enzyme complexed to a first plurality of guide oligonucleotides to remove a first plurality of immobilized amplification products, wherein the first fraction includes the complement of the first plurality of guide oligonucleotides. In embodiments, the method further includes: i) contacting a second fraction of the cleavable sites with a second plurality of TtAgo enzyme complexed to a second plurality of guide oligonucleotides to remove a second plurality of immobilized amplification products, wherein the second fraction includes the complement of the second plurality of guide oligonucleotides. In embodiments, the method further includes: ii) repeating step i) for one or more additional fractions of cleavable sites to remove one or more additional pluralities of immobilized amplification products.

In embodiments, following step f) (i.e., the formation a plurality of immobilized amplification products on the solid support), each of the plurality of amplification products includes 1, 2, or 3 template polynucleotide sequences. In embodiments, following step f), each of the plurality of amplification products includes 1 or 2 template polynucleotide sequences. In embodiments, following step f), each of the plurality of amplification products includes 1 template polynucleotide sequence. In embodiments, following step f), each of the plurality of amplification products includes less than 5, less than 4, less than 3, or less than 2 template polynucleotide sequences.

In another aspect is provided a method of amplifying a template polynucleotide on a solid support. In embodiments, the method includes (i) executing one or more amplification cycles thereby forming a plurality of immobilized amplification products including a cleavable site on the solid support, wherein each amplification cycle includes: a) hybridizing the template polynucleotide to a first oligonucleotide, wherein the first oligonucleotide includes a cleavable site and is attached to the solid support, and extending the first oligonucleotide with a polymerase to generate an immobilized complement of the template polynucleotide; b) denaturing the template polynucleotide and immobilized complement; c) hybridizing the immobilized complement to a second oligonucleotide, wherein the second oligonucleotide includes a cleavable site and is attached to the solid support, and extending the second oligonucleotide with a polymerase to generate an immobilized copy of the template polynucleotide; and (ii) removing a fraction of the plurality of immobilized amplification products; and (iii) after step (ii), executing one or more amplification cycles. In embodiments, the method does not include sequencing the amplification products prior to step (iii). In embodiments, the method further includes repeating steps (ii) and (iii).

In embodiments, prior to step (i), the method includes executing one or more sparse-seed cycles, wherein each sparse-seed cycle includes contacting the solid support with a plurality of template polynucleotides and forming a plurality of template complexes, wherein each template complex includes a template polynucleotide hybridized to an immobilized oligonucleotide including a cleavable site; contacting the template complexes with a polymerase and extending the immobilized oligonucleotide to form a plurality of extended complements of templates; and removing a fraction of the extended complements of templates. Embodiments of sparse-seed cycles are illustrated in FIG. 4, where two cycles of sparse-seed cycles are shown. FIG. 4A illustrates a sparse-seed cycle, where templates hybridize to the immobilized primers with cleavable sites and are extended by a polymerase, which is followed by the removal of a fraction of the immobilized complements of the templates (e.g., via digestion). FIG. 4B illustrates another cycle sparse-seed cycle, where the templates are contacted with the solid support again to hybridize with the immobilized primers, followed by primer extension by a polymerase and removal of a fraction of the extended complements of the templates (e.g., via digestion). Following removal of fractions of the extended complements of the templates from cycles of sparse-seeding, one or more amplification cycles are again executed, as shown in FIGS. 4B and 4C.

In embodiments, the method includes 1 sparse-seed cycle. In embodiments, the method includes 2 sparse-seed cycles. In embodiments, the method includes 3 sparse-seed cycles. In embodiments, the method includes 4 sparse-seed cycles. In embodiments, the method includes 5 sparse-seed cycles. In embodiments, the method includes 6 sparse-seed cycles. In embodiments, the method includes 7 sparse-seed cycles. In embodiments, the method includes 8 sparse-seed cycles. In embodiments, the method includes 9 sparse-seed cycles. In embodiments, the method includes 10 sparse-seed cycles. In embodiments, the method includes 2 to 8 sparse-seed cycles. In embodiments, the method includes 10 to 20 sparse-seed cycles. In embodiments, the method includes 20 to 30 sparse-seed cycles. In embodiments, the method includes 30 to 40 sparse-seed cycles. In embodiments, the method includes 40 to 50 sparse-seed cycles. In embodiments, the method includes 40 or more sparse-seed cycles. In embodiments, one or more amplification cycles occur after a sparse-seed cycle.

Primer extension products from an earlier cycle may serve as templates for a later amplification cycle. In embodiments, prior to step (ii), the method further includes 2 to 50 amplification cycles. In embodiments, prior to step (ii), the method further includes 2-5 amplification cycles. In embodiments, prior to step (ii), the method further includes 5-15 amplification cycles. In embodiments, prior to step (ii), the method further includes 2 to 20 amplification cycles. In embodiments, prior to step (ii), the method further includes 10 to 20 amplification cycles. In embodiments, prior to step (ii), the method further includes 20 to 30 amplification cycles. In embodiments, prior to step (ii), the method further includes 30 to 40 amplification cycles. In embodiments, prior to step (ii), the method further includes 40 to 50 amplification cycles. In embodiments, prior to step (ii), the method further includes 40 or more amplification cycles.

In embodiments, step (ii) further includes incubating the fraction of cleavable sites with the cleaving agent for between about 5 seconds to about 30 minutes. In embodiments, step (ii) further includes incubating the fraction of cleavable sites with the cleaving agent for about 5 seconds to about 30 seconds, about 30 seconds to about 60 seconds, about 1 minute to about 5 minutes, about 5 minutes to about 15 minutes, or about 15 minutes to about 30 minutes. In embodiments, step (ii) further includes incubating the fraction of cleavable sites with the cleaving agent for about 5 seconds, about 10 seconds, about 20 seconds, about 30 seconds, about 1 minutes, about 2 minutes, about 3 minutes, about 4 minutes, about 5 minutes, about 10 minutes, about 15 minutes, about 20 minutes, about 25 minutes, about 30 minutes, about 45 minutes, or about 60 minutes. In embodiments, step (ii) further includes incubating the fraction of cleavable sites with the cleaving agent for between about 1 hour to 2 hours, between about 2 hours to 4 hours, between about 4 hours to 8 hours, between about 8 hours to 12 hours, between about 12 hours to 16 hours, or between about 16 hours to 24 hours. In embodiments, step (ii) further includes incubating the fraction of cleavable sites with the cleaving agent for between about 1, 2, 3, 4, 5, 6, 7, 8, 9, 10, 11, 12, 13, 14, 15, 16, 17, 18, 19, 20, 21, 22, 23, or 24 hours. In embodiments, step (ii) further includes incubating the fraction of cleavable sites with the cleaving agent for more than 24 hours.

In embodiments, step (ii) includes contacting less than 100% of the cleavable sites with a cleaving agent. In embodiments, step (ii) includes contacting about 90%, about 91%, about 92%, about 93%, about 94%, about 95%, about 96%, about 97%, about 98%, or about 99% of the cleavable sites with the cleaving agent. In embodiments, step (ii) includes contacting about 95%, about 96%, about 97%, about 98%, or about 99% of the cleavable sites with the cleaving agent. In embodiments, step (ii) includes contacting about 99% of the cleavable sites with the cleaving agent. In embodiments, step (ii) includes contacting about 99%, about 99.1%, about 99.2%, about 99.3%, about 99.4% or about 99.5% of the cleavable sites with the cleaving agent. In embodiments, step (ii) includes contacting about 80% of the cleavable sites with the cleaving agent. In embodiments, step (ii) includes contacting 80%, 85%, 90% or more of the cleavable sites with the cleaving agent. In embodiments, step (ii) includes contacting greater than 90% of the cleavable sites with the cleaving agent.

In embodiments, step (ii) includes contacting a first fraction of the cleavable sites with a first plurality of TtAgo enzyme complexed to a first plurality of guide oligonucleotides to remove a first plurality of immobilized amplification products, wherein the first fraction includes the complement of the first plurality of guide oligonucleotides. In embodiments, the method further includes: 1) contacting a second fraction of the cleavable sites with a second plurality of TtAgo enzyme complexed to a second plurality of guide oligonucleotides to remove a second plurality of immobilized amplification products, wherein the second fraction includes the complement of the second plurality of guide oligonucleotides. In embodiments, the method further includes: 2) repeating step 1) for one or more additional fractions of cleavable sites to remove one or more additional pluralities of immobilized amplification products.

In embodiments, after step (ii), the method further includes 2 to 50 amplification cycles. In embodiments, after step (ii), the method further includes 2-5 amplification cycles. In embodiments, after step (ii), the method further includes 5-15 amplification cycles. In embodiments, after step (ii), the method further includes 2 to 20 amplification cycles. In embodiments, after step (ii), the method further includes 10 to 20 amplification cycles. In embodiments, after step (ii), the method further includes 20 to 30 amplification cycles. In embodiments, after step (ii), the method further includes 30 to 40 amplification cycles. In embodiments, after step (ii), the method further includes 40 to 50 amplification cycles. In embodiments, after step (ii), the method further includes 40 or more amplification cycles.

In embodiments, following step (iii), each of the plurality of amplification products includes 1, 2, or 3 template polynucleotide sequences. In embodiments, following step (iii), each of the plurality of amplification products includes 1 or 2 template polynucleotide sequences. In embodiments, following step (iii), each of the plurality of amplification products includes 1 template polynucleotide sequence. In embodiments, following step (iii), each of the plurality of amplification products includes less than 5, less than 4, less than 3, or less than 2 template polynucleotide sequences.

In an aspect is provided a method of amplifying a template polynucleotide on a solid support. In embodiments, the method includes: a) hybridizing the template polynucleotide to a first oligonucleotide, wherein said first oligonucleotide comprises a cleavable site and is attached to the solid support, and extending with a polymerase the first oligonucleotide to generate an immobilized complement of the template polynucleotide; b) removing (e.g., denaturing) the template polynucleotide; c) hybridizing the immobilized complement of the template polynucleotide to a second oligonucleotide, wherein said second oligonucleotide comprises a cleavable site and is attached to the solid support, and extending with a polymerase the second oligonucleotide to generate an immobilized copy of the template polynucleotide; d) repeating steps b) and c) one or more times, thereby forming a plurality of immobilized amplification products on the solid support, each amplification product comprises said cleavable site; and e) contacting a fraction of the cleavable sites with a cleaving agent to remove a fraction of the plurality of immobilized amplification products. In embodiments, the method further includes repeating step e). In embodiments, step e) occurs prior to steps c) and d). In embodiments, the method further includes repeating steps a)-e) one or more times. In embodiments, the method further includes repeating steps a)-d) one or more times. In embodiments, steps a) and b) are repeated one or more times. Repeating steps, such as step e), where step e includes contacting a fraction of the cleavable sites with a cleaving agent, 1 or more times means that the number of steps to be performed is one, and the number of steps is increased.

In embodiments, the method further includes repeating step e) (i.e., contacting a fraction of the cleavable sites with a cleaving agent to remove a fraction of the plurality of amplification products). In embodiments, the method further includes repeating step e) one or more times. In embodiments, the method further includes repeating step e) 1, 2, 3, 4, 5, 6, 7, 8, 9, 10, or more times. In embodiments, the method further includes repeating step e) 10 or more times. In embodiments, the method further includes repeating step e) 15, 20, 25, 30, 35, or more times. In embodiments, the method further includes repeating steps d) and e). Repeating steps (e.g., step e)) 1 or more times means that the number of steps to be performed is one, and the number of steps is increased. In embodiments, repeating step e) 2 times means contacting a fraction of the cleavable sites for the first time with a cleaving agent to remove a fraction of the plurality of immobilized amplification products, which would then be followed by the hybridization of the template polynucleotide with a cleavable site to an oligonucleotide immobilized onto the solid support to generate an immobilized complement of the template polynucleotide, removal the template polynucleotide, hybridization of the immobilized complement of the template polynucleotide to a second immobilized oligonucleotide to facilitate generating a plurality of immobilized amplification products with cleavable sites, and contacting the fraction of the cleavable sites for a second time with a cleaving agent to remove a fraction of the plurality of immobilized amplification products. FIG. 4A depicts contacting a fraction of the cleavable sites for the first time with a cleaving agent to remove a fraction of the plurality of immobilized amplification products, and FIG. 4B illustrates contacting a fraction of the cleavable sites on the same solid support for the second time with a cleaving agent to remove a fraction of the plurality of immobilized amplification products.

In embodiments, step e) is repeated 1 to about 10 times in total. In embodiments, step b) is repeated between 10 to about 15 times. In embodiments, step e) is repeated 1, 2, 3, 4, 5, 6, 7, 8, 9, or 10 times. In embodiments, step e) is repeated 10, 11, 12, 13, 14, or 15 times. In embodiments, step e) is repeated 5 or more times. In embodiments, step e) is repeated 10 or more times. In embodiments, step e) is repeated more than 15 times.

In embodiments, step e) further includes incubating the fraction of cleavable sites with the cleaving agent for between about 5 seconds up to about 30 minutes. In embodiments, step e) further includes incubating the fraction of cleavable sites with the cleaving agent for about 5 seconds to about 30 seconds, about 30 seconds to about 60 seconds, about 1 minute to about 5 minutes, about 5 minutes to about 15 minutes, or about 15 minutes to about 30 minutes. In embodiments, step e) further includes incubating the fraction of cleavable sites with the cleaving agent for about 5 seconds, about 10 seconds, about 20 seconds, about 30 seconds, about 1 minutes, about 2 minutes, about 3 minutes, about 4 minutes, about 5 minutes, about 10 minutes, about 15 minutes, about 20 minutes, about 25 minutes, about 30 minutes, about 45 minutes, or about 60 minutes. In embodiments, step e) further includes incubating the fraction of cleavable sites with the cleaving agent for between about 1 hour to 2 hours, between about 2 hours to 4 hours, between about 4 hours to 8 hours, between about 8 hours to 12 hours, between about 12 hours to 16 hours, or between about 16 hours to 24 hours. In embodiments, step e) further includes incubating the fraction of cleavable sites with the cleaving agent for between about 1, 2, 3, 4, 5, 6, 7, 8, 9, 10, 11, 12, 13, 14, 15, 16, 17, 18, 19, 20, 21, 22, 23, or 24 hours. In embodiments, step e) further includes incubating the fraction of cleavable sites with the cleaving agent for more than 24 hours.

In embodiments, step e) includes contacting less than 100% of the cleavable sites with a cleaving agent. In embodiments, step e) includes contacting about 90%, about 91%, about 92%, about 93%, about 94%, about 95%, about 96%, about 97%, about 98%, or about 99% of the cleavable sites with the cleaving agent. In embodiments, step e) includes contacting about 95%, about 96%, about 97%, about 98%, or about 99% of the cleavable sites with the cleaving agent. In embodiments, step e) includes contacting about 99% of the cleavable sites with the cleaving agent. In embodiments, step e) includes contacting about 99%, about 99.1%, about 99.2%, about 99.3%, about 99.4% or about 99.5% of the cleavable sites with the cleaving agent. In embodiments, step e) includes contacting about 80% of the cleavable sites with a cleaving agent. In embodiments, step e) includes contacting 80%, 85%, 90% or more of the cleavable sites with the cleaving agent. In embodiments, step e) includes contacting greater than 90% of the cleavable sites with the cleaving agent.

In embodiments, step e) includes contacting a first fraction of the cleavable sites with a first plurality of TtAgo enzyme complexed to a first plurality of guide oligonucleotides to remove a first plurality of immobilized amplification products, wherein the first fraction includes the complement of the first plurality of guide oligonucleotides. In embodiments, the method further includes: i) contacting a second fraction of the cleavable sites with a second plurality of TtAgo enzyme complexed to a second plurality of guide oligonucleotides to remove a second plurality of immobilized amplification products, wherein the second fraction includes the complement of the second plurality of guide oligonucleotides. In embodiments, the method further includes: ii) repeating step i) for one or more additional fractions of cleavable sites to remove one or more additional pluralities of immobilized amplification products.

In embodiments, following step d), each of the plurality of amplification products includes 1, 2, or 3 template polynucleotide sequences. In embodiments, following step d), each of the plurality of amplification products includes 1 or 2 template polynucleotide sequences. In embodiments, following step d), each of the plurality of amplification products includes 1 template polynucleotide sequence. In embodiments, following step d), each of the plurality of amplification products includes less than 5, less than 4, less than 3, or less than 2 template polynucleotide sequences.

In an aspect is provided a method of amplifying a template polynucleotide on a solid support. In embodiments, the method includes: a) hybridizing the template polynucleotide to a first oligonucleotide, wherein the first oligonucleotide includes a cleavable site and is attached to the solid support, and extending with a polymerase the first oligonucleotide to generate an immobilized complement of the template polynucleotide; b) removing (e.g., denaturing) the template polynucleotide and hybridizing the immobilized complement of the template polynucleotide to a second oligonucleotide, wherein the second oligonucleotide includes a cleavable site and is attached to the solid support, and extending with a polymerase the second oligonucleotide to generate an immobilized copy of the template polynucleotide; and c) repeating steps a) and b) one or more times, thereby forming a plurality of immobilized amplification products on the solid support, where each amplification product includes the cleavable site. In embodiments, the method further includes d) contacting a fraction of the cleavable sites with a cleaving agent to remove a fraction of the plurality of amplification products. In embodiments, the method further includes e) repeating steps a) and b) one or more times. Repeating steps, such as step d), where step d includes contacting a fraction of the cleavable sites with a cleaving agent, 1 or more times means that the number of steps to be performed is one, and the number of steps is increased.

In an aspect is provided a method of amplifying a template polynucleotide on a solid support, the method including: a) hybridizing the template polynucleotide to a first oligonucleotide, wherein the first oligonucleotide includes a cleavable site and is attached to the solid support, and extending with a polymerase the first oligonucleotide to generate an immobilized complement of the template polynucleotide; b) removing (e.g., denaturing) the template polynucleotide and hybridizing the immobilized complement of the template polynucleotide to a second oligonucleotide, wherein the second oligonucleotide includes a cleavable site and is attached to the solid support, and extending with a polymerase the second oligonucleotide to generate an immobilized copy of the template polynucleotide; c) repeating steps a) and b) one or more times, thereby forming a plurality of immobilized amplification products on the solid support, each amplification product includes the cleavable site; and d) contacting a fraction of the cleavable sites with a cleaving agent to remove a fraction of the plurality of amplification products. In embodiments, the method further includes e) repeating steps a) and b) one or more times.

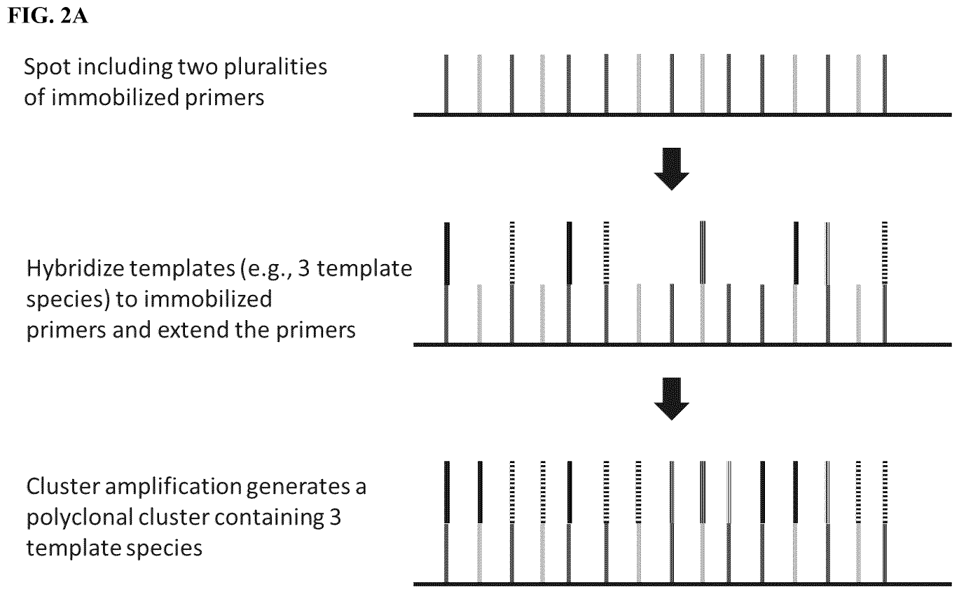

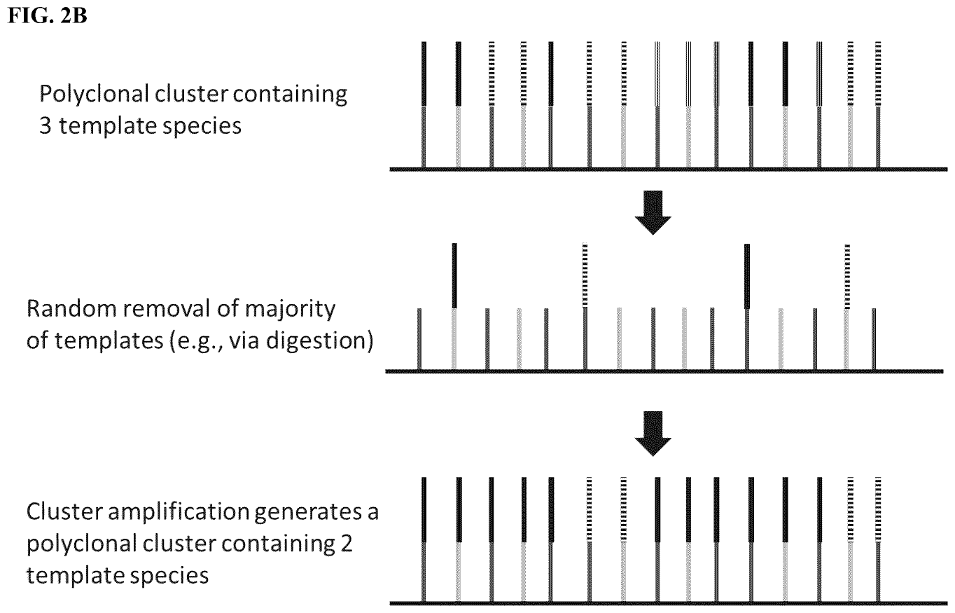

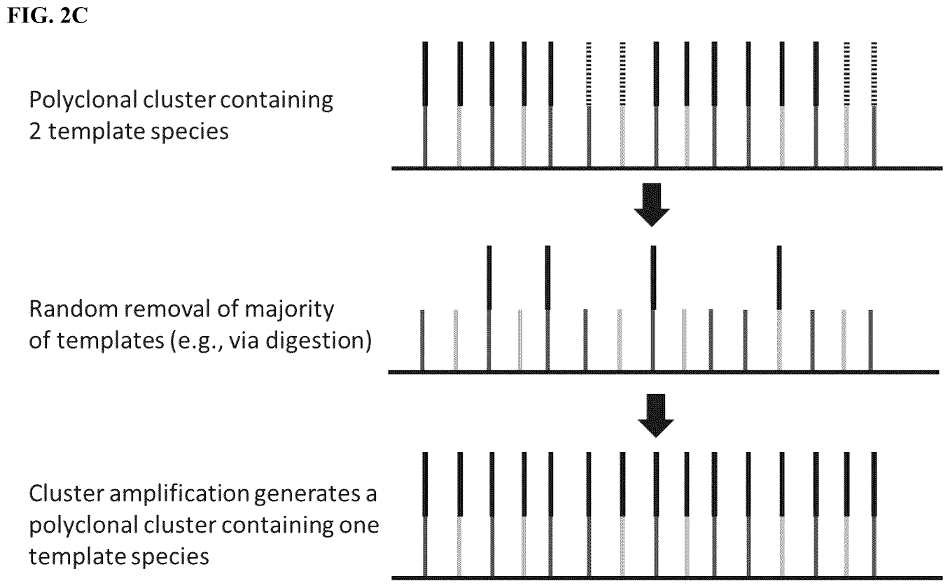

In embodiments, the method further includes repeating step d) (i.e., contacting a fraction of the cleavable sites with a cleaving agent to remove a fraction of the plurality of amplification products). In embodiments, the method further includes repeating step d) one or more times. In embodiments, the method further includes repeating step d) 1, 2, 3, 4, 5, 6, 7, 8, 9, 10, or more times. In embodiments, the method further includes repeating step d) 10 or more times. In embodiments, the method further includes repeating step d) 15, 20, 25, 30, 35, or more times. In embodiments, the method further includes repeating steps c) and d). In embodiments, the method further includes repeating steps c) and d), wherein step d) is repeated one or more times. In embodiments, the method further includes repeating steps c) and d), wherein step d) is repeated 1, 2, 3, 4, 5, 6, 7, 8, 9, 10, or more times. In embodiments, the method further includes repeating steps c) and d), wherein step d) is repeated 15, 20, 25, 30, 35, or more times. Repeating steps (e.g., steps c) and d)) 1 or more times means that the number of steps to be performed is one, and the number of steps is increased. In embodiments, repeating steps c) and d) 2 times means performing cluster amplification, followed by contacting the fraction of cleavable sites with a cleaving agent to remove a fraction of the plurality of immobilized amplification products as shown in FIGS. 2A and 2B, and these steps would be subsequently repeated for a second time by performing cluster amplification again, followed by contacting the fraction of cleavable sites with a cleaving agent, as shown in FIGS. 2B and 2C.

In embodiments, step d) is repeated 1 to about 10 times. In embodiments, step d) is repeated between 10 to about 15 times. In embodiments, step d) is repeated 1, 2, 3, 4, 5, 6, 7, 8, 9, or 10 times. In embodiments, step d) is repeated 10, 11, 12, 13, 14, or 15 times. In embodiments, step d) is repeated 5 or more times. In embodiments, step d) is repeated 10 or more times. In embodiments, step d) is repeated more than 15 times.

In embodiments, step d) further includes incubating the fraction of cleavable sites with the cleaving agent for between about 5 seconds up to about 30 minutes. In embodiments, step d) further includes incubating the fraction of cleavable sites with the cleaving agent for about 5 seconds to about 30 seconds, about 30 seconds to about 60 seconds, about 1 minute to about 5 minutes, about 5 minutes to about 15 minutes, or about 15 minutes to about 30 minutes. In embodiments, step d) further includes incubating the fraction of cleavable sites with the cleaving agent for about 5 seconds, about 10 seconds, about 20 seconds, about 30 seconds, about 1 minutes, about 2 minutes, about 3 minutes, about 4 minutes, about 5 minutes, about 10 minutes, about 15 minutes, about 20 minutes, about 25 minutes, about 30 minutes, about 45 minutes, or about 60 minutes. In embodiments, step d) further includes incubating the fraction of cleavable sites with the cleaving agent for between about 1 hour to 2 hours, between about 2 hours to 4 hours, between about 4 hours to 8 hours, between about 8 hours to 12 hours, between about 12 hours to 16 hours, or between about 16 hours to 24 hours. In embodiments, step d) further includes incubating the fraction of cleavable sites with the cleaving agent for between about 1, 2, 3, 4, 5, 6, 7, 8, 9, 10, 11, 12, 13, 14, 15, 16, 17, 18, 19, 20, 21, 22, 23, or 24 hours. In embodiments, step d) further includes incubating the fraction of cleavable sites with the cleaving agent for more than 24 hours.

In embodiments, step d) includes contacting less than 100% of the cleavable sites with a cleaving agent. In embodiments, step d) includes contacting about 90%, about 91%, about 92%, about 93%, about 94%, about 95%, about 96%, about 97%, about 98%, or about 99% of the cleavable sites with the cleaving agent. In embodiments, step d) includes contacting about 95%, about 96%, about 97%, about 98%, or about 99% of the cleavable sites with the cleaving agent. In embodiments, step d) includes contacting about 99% of the cleavable sites with the cleaving agent. In embodiments, step d) includes contacting about 99%, about 99.1%, about 99.2%, about 99.3%, about 99.4% or about 99.5% of the cleavable sites with the cleaving agent. In embodiments, step d) includes contacting about 80% of the cleavable sites with the cleaving agent. In embodiments, step d) includes contacting 80%, 85%, 90% or more of the cleavable sites with the cleaving agent. In embodiments, step d) includes contacting greater than 90% of the cleavable sites with the cleaving agent.

In embodiments, the template polynucleotide is in solution (e.g., a buffered solution) prior to step a). In embodiments, the template polynucleotide is immobilized on the solid support prior to step a) via a covalent attachment at the 5′ end of the template polynucleotide. In embodiments, the template polynucleotide is annealed to a complementary sequence on the solid support prior to step a).

In embodiments, following step e), each of the plurality of amplification products includes 1, 2, or 3 template polynucleotide sequences. In embodiments, following step e), each of the plurality of amplification products includes 1 or 2 template polynucleotide sequences. In embodiments, following step e), each of the plurality of amplification products includes 1 template polynucleotide sequence. In embodiments, following step e), each of the plurality of amplification products includes less than 5, less than 4, less than 3, or less than 2 template polynucleotide sequences.

In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. The cleavable site(s) may be deoxyuracil triphosphate (dUTP), deoxy-8-Oxo-guanine triphosphate (d-8-oxoG), or other modified nucleotide(s), such as those described, for example, in US 2012/0238738, which is incorporated herein by reference for all purposes. In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence (e.g., a sequence containing a modified or unmodified nucleotide, or a motif recognized by a cleaving enzyme) specifically recognized by a cleaving agent. In embodiments, the cleavable site includes a sequence specifically recognized by a restriction enzyme. In embodiments, the cleavable site includes one or more ribonucleotides. In embodiments, the cleavable site includes 2 to 5 ribonucleotides. In embodiments, the cleavable site includes one ribonucleotide. In embodiments, the cleavable sites can be cleaved at or near a modified nucleotide or bond by enzymes or chemical reagents, collectively referred to herein as “cleaving agents.”

In embodiments, contacting a fraction of the cleavable sites with a cleaving agent removes about 90%, about 91%, about 92%, about 93%, about 94%, about 95%, about 96%, about 97%, about 98%, or about 99% of the plurality of amplification products. In embodiments, the cleaving activity removes about 95%, about 96%, about 97%, about 98%, or about 99% of the plurality of amplification products. In embodiments, the cleaving activity removes about 99% of the plurality of amplification products.

In embodiments, the cleaving agent includes a reducing agent, sodium periodate, Rnase, Formamidopyrimidine DNA Glycosylase (Fpg), endonuclease, or uracil DNA glycosylase (UDG). Examples of cleaving agents include DNA repair enzymes, glycosylases, DNA cleaving endonucleases, or ribonucleases. For example, cleavage at dUTP may be achieved using uracil DNA glycosylase and endonuclease VIII (USER™, NEB, Ipswich, Mass.), as described in U.S. Pat. No. 7,435,572. In embodiments, when the modified nucleotide is a ribonucleotide, the cleavable site can be cleaved with an endoribonuclease. In embodiments, cleaving an extension product includes contacting the cleavable site with a cleaving agent, wherein the cleaving agent includes a reducing agent, sodium periodate, RNase, formamidopyrimidine DNA glycosylase (Fpg), endonuclease, restriction enzyme, or uracil DNA glycosylase (UDG). In embodiments, the cleaving agent is an endonuclease enzyme such as nuclease P1, AP endonuclease, T7 endonuclease, T4 endonuclease IV, Bal 31 endonuclease, Endonuclease I (endo I), Micrococcal nuclease, Endonuclease II (endo VI, exo III), nuclease BAL-31 or mung bean nuclease. In embodiments, the cleaving agent includes a restriction endonuclease, including, for example a type IIS restriction endonuclease. In embodiments, the cleaving agent is an exonuclease (e.g., RecBCD), restriction nuclease, endoribonuclease, exoribonuclease, or RNase (e.g., RNAse I, II, or III). In embodiments, the cleaving agent is a restriction enzyme. In embodiments, the cleaving agent includes a glycosylase and one or more suitable endonucleases. In embodiments, cleavage is performed under alkaline (e.g., pH greater than 8) buffer conditions at between 40° C. to 80° C. In embodiments, the restriction enzyme recognition sequence included in the cleavable site is selected to be a “rare-cutting” restriction enzyme recognition sequence, e.g., a restriction enzyme that cuts with low frequency in any given genome. For example, Nod is a rare cutter with an eight-base recognition site, which will occur on average about once every 65,000 base pairs in a genome (assuming an average frequency of each type of canonical base of ¼). Other rare-cutting enzymes are known in the art and commercially available, including AbsI, AscI, BbvCI, CciNI, FseI, MreI, PaIAI, RigI, SdaI, and SgsI.

In some embodiments, the cleaving agent includes one or more restriction endonucleases. When employing restriction endonucleases for cleavage, careful selection of the restriction endonuclease is beneficial, given the need for high efficiency cleavage and the fact that efficiency of cleavage can vary significantly according to the specific restriction endonuclease. Using a novel single molecule counting approach, Zhang et al (see, Zhang Y et al. PLoS ONE. 2020. 15(12): e0244464, which is incorporated herein by reference in its entirety) precisely determined the cleavage efficiency of a variety of common restriction enzymes and the CRISPR-Cas9 nuclease. Zhang reported single enzyme digestion efficiencies ranging from as low as 67.12% for NdeI to as high as 99.53% for EcoRI-HF. Importantly, Zhang notes that the duration of digestion has minimal effect on the overall digestion efficiency such that the fraction of digested templates is nearly unchanged after the first 5 minutes of incubation, suggesting that a 5-minute incubation time serves as a reasonable starting point for optimization of many candidate restriction endonucleases. In embodiments, the fraction of immobilized amplification products removed is in part controlled by the duration of incubation and/or the concentration of the cleaving agent.

In embodiments, the cleaving agent includes a single restriction endonuclease. In embodiments, the restriction endonuclease may include XbaI, EcoRI-HF, NheI, BamHI, XcmI, PflMI, BstEII, NcoI, HpaI, BsgI, AfeI, StuI, BsrGI, or a CRISPR-Cas9 nuclease (e.g., to achieve an approximate 95% cleavage or digestion rate, or the cleaving activity). In embodiments, the restriction endonuclease may include XbaI, EcoRI, BamHI, XcmI or BstEII (e.g., to achieve an approximate 98% or greater cleavage or digestion rate, or the cleaving activity). In embodiments, the restriction endonuclease may include EcoRI or XbaI (e.g., to achieve an approximate 99% or greater cleavage or digestion rate, or the cleaving activity). In some embodiments, the efficiency of cleavage may be further improved by inclusion of more than one restriction enzyme recognition site between the adapter (e.g., adapter including a platform primer binding sequence and/or sequencing primer binding sequence) and insert sequence. In some embodiments, multiple restriction endonucleases may be used in combination to precisely tune the cleavage efficiency. For example, in embodiments where >99.5% cleavage efficiency is required, a suitable dual restriction endonuclease cleavage solution may include XbaI (99.25% efficiency, as reported in Zhang) and NdeI (67.12% efficiency, as reported in Zhang), while the library constructs contain recognition sites for both XbaI and NdeI. Here, the estimated combined cleavage efficiency of the dual restriction endonuclease system is approximately 1-(1-.9925)(1-.6712)=99.75%.

In embodiments, the cleaving agent is a programmable endonuclease. In embodiments, the cleaving agent is a programmable endonucleasecomplex. In embodiments, the programmable endonuclease is Thermus thermophilus argonaute (TtAgo) enzyme, or a mutant thereof. In embodiments, the programmable endonuclease is from the haloalkaliphilic archaebacterium N. gregoryi SP2 (NgAgo) or a modification or homolog thereof. Additional argonaute proteins are described, for example, in U.S. Pat. Pubs. 2015/0089681 and 2021/0189388, each of which is incorporated herein by reference in its entirety.

In embodiments, the method further includes a guide oligonucleotide in complex with a TtAgo enzyme (e.g., a TtAgo enzyme or a mutant thereof). In embodiments, the programmable endonuclease further includes a guide oligonucleotide. In embodiments, the guide oligonucleotide includes a first targeting domain and a second targeting domain, wherein the first targeting domain and second targeting domain are complementary to a sequence of the cleavable site. In embodiments, the first targeting domain is complementary to a portion of a platform primer sequence, and the second targeting domain is complementary to a portion of a sequencing primer binding sequence.

In embodiments, step (d) includes contacting a first fraction of the cleavable sites with a first plurality of TtAgo enzyme complexed to a first plurality of guide oligonucleotides to remove a first plurality of immobilized amplification products, wherein the first fraction includes the complement of the first plurality of guide oligonucleotides. In embodiments, the method further includes: i) contacting a second fraction of the cleavable sites with a second plurality of TtAgo enzyme complexed to a second plurality of guide oligonucleotides to remove a second plurality of immobilized amplification products, wherein the second fraction includes the complement of the second plurality of guide oligonucleotides. In embodiments, the method further includes: ii) repeating step i) for one or more additional fractions of cleavable sites to remove one or more additional pluralities of immobilized amplification products.

In embodiments, cleavage by the programmable endonuclease complex generates an immobilized platform primer with a free 3′-OH. In embodiments, cleavage by the TtAgo complex generates an immobilized platform primer with a free 3′-OH. In embodiments, the method further includes annealing the immobilized platform primer with the free 3′-OH (e.g., the cleaved immobilized platform primer) to an immobilized template polynucleotide, or complement thereof, and extending the annealed immobilized platform primer with a polymerase to generate an amplification product.

In embodiments, the 3′ terminal nucleotide of the immobilized platform primer sequence is complementary to the 5′ terminal nucleotide of the first targeting domain of the guide oligonucleotide.

During optimization of cleavage by the cleaving agent, it may be useful to estimate the cleavage efficiency at single molecule resolution. FIG. 12 depicts an exemplary strategy for determining the cleavage efficiency at single molecule resolution using a patterned next generation sequencing flow cell. A patterned flow cell is contacted with a predetermined number of template molecules to produce a plurality of spots seeded each with a single template molecule following a Poisson distribution. Following seeding, amplification clusters are generated, and the number of clusters are quantified (e.g., by fluorescence microscopy) to obtain the baseline number of clusters. The process is repeated, this time with the addition of a cleavage step (e.g., via restriction endonuclease cleavage) prior to clustering and counting. The number of clusters detected following cleavage is then compared to the baseline number to determine the cleavage efficiency.

In embodiments, cleaving includes maintaining suitable reaction conditions to permit efficient cleavage (e.g., buffer, pH, temperature conditions). In embodiments, cleaving is performed at about 20° C. to about 60° C. In embodiments, cleavage is performed at about 20° C. to about 30° C., about 30° C. to about 40° C., about 40° C. to about 50° C., or about 50° C. to about 60° C. In embodiments, cleavage is performed at about 20° C., about 25° C., about 30° C., about 35° C., about 37° C., about 40° C., about 42° C., about 45° C., about 48° C., about 50° C., about 55° C., or about 60° C. In embodiments, cleavage is performed at less than 20° C. In embodiments, cleavage is performed at greater than 60° C.

In embodiments, cleavage is performed for about 5 seconds (sec) to about 24 hours (hrs). In embodiments, cleavage is performed for about 5 sec to about 30 sec, about 30 sec to about 60 sec, about 1 minute (min) to about 5 min, about 5 min to about 15 min, about 15 min to about 30 min, about 30 min to about 60 min, about 1 hr to about 4 hrs, about 4 hrs to about 12 hrs, or about 12 hrs to about 24 hrs. In embodiments, cleavage is performed for about 5 sec, 15 sec, 30 sec, 45 sec, 1 min, 2 min, 3 min, 4 min, 5 min, 6 min, 7 min, 8 min, 9 min, 10 min, 11 min, 12 min, 13 min, 14 min, or about 15 min. In embodiments, cleavage is performed for about 20 min, 25 min, 30 min, 35 min, 40 min, 45 min, 50 min, 55 min, or about 1 hr. In embodiments, cleavage is performed for about 2 hrs, 3 hrs, 4 hrs, 5 hrs, 6 hrs, 7 hrs, 8 hrs, 9 hrs, 10 hrs, 11 hrs, or about 12 hrs. In embodiments, cleavage is performed for about 14 hrs, 16 hrs, 18 hrs, 20 hrs, 22 hrs, or about 24 hrs.

In embodiments, cleavage is performed with about 1 unit (U) to about 50 U of restriction endonuclease. The term “unit (U)” or “enzyme unit (U)” is used in accordance with its plain and ordinary meaning, and refers to the amount of the enzyme that catalyzes the conversion of one micromole of substrate per minute under the specified conditions of a given assay. In embodiments, cleavage is performed with about 1 U to about 5 U of restriction endonuclease. In embodiments, cleavage is performed with about 5 U to about 10 U of restriction endonuclease. In embodiments, cleavage is performed with about 10 U to about 15 U of restriction endonuclease. In embodiments, cleavage is performed with about 15 U to about 20 U of restriction endonuclease. In embodiments, cleavage is performed with about 20 U to about 25 U of restriction endonuclease. In embodiments, cleavage is performed with about 25 U to about 35 U of restriction endonuclease. In embodiments, cleavage is performed with about 35 U to about 50 U of restriction endonuclease. In embodiments, cleavage is performed with about 1, 2, 3, 4, 5, 6, 7, 8, 9, 10, 11, 12, 13, 14, 15, 16, 17, 18, 19, 20, 21, 22, 23, 24, 25, 30, 35, 40, 45 or 50 U of restriction endonuclease. In embodiments, cleavage is performed with less than about 1 U of restriction endonuclease. In embodiments, cleavage is performed with greater than about 50 U of restriction endonuclease.

In embodiments, the solid support includes a plurality of oligonucleotides, wherein each oligonucleotide is attached to the solid support at a 5′ end of the oligonucleotide (i.e., the solid support includes a plurality of immobilized oligonucleotides). In embodiments, each oligonucleotide is attached to the solid support via a linker. The linker may also include spacer nucleotides. Including spacer nucleotides in the linker puts the polynucleotide in an environment having a greater resemblance to free solution. This can be beneficial, for example, in enzyme-mediated reactions such as sequencing-by-synthesis. It is believed that such reactions suffer less steric hindrance issues that can occur when the polynucleotide is directly attached to the solid support or is attached through a very short linker (e.g., a linker comprising about 1 to 3 carbon atoms). Spacer nucleotides form part of the polynucleotide but do not participate in any reaction carried out on or with the polynucleotide (e.g. a hybridization or amplification reaction). In embodiments, the spacer nucleotides include 1 to 20 nucleotides. In embodiments, the linker includes 10 spacer nucleotides. In embodiments, the linker includes 12 spacer nucleotides. In embodiments, the linker includes 15 spacer nucleotides. It is preferred to use polyT spacer nucleotides, although other nucleotides and combinations thereof can be used. In embodiments, the linker includes 10, 11, 12, 13, 14, or 15 dT spacer nucleotides. In embodiments, the linker includes 12 dT spacer nucleotides. Spacer nucleotides are typically included at the 5′ ends of polynucleotides which are attached to a suitable support. Attachment can be achieved via a phosphorothioate present at the 5′ end of the polynucleotide, an azide moiety, a dibenzocyclooctyne (DBCO) moiety, or any other bioconjugate reactive moiety. The linker may be a carbon-containing chain such as those of formula —(CH₂)ₙ— wherein “n” is from 1 to about 1000. However, a variety of other linkers may be used so long as the linkers are stable under conditions used in DNA sequencing. In embodiments, the linker includes polyethylene glycol (PEG) having a general formula of —(CH₂—CH₂—O)ₘ—, wherein m is from about 1 to 500. In embodiments, m is 8 to 24. In embodiments, m is 10 to 12. In embodiments, the linker, or the immobilized oligonucleotides (e.g., primers) include a cleavable site. In embodiments, a cleavable site is a location which allows controlled cleavage of the immobilized polynucleotide strand (e.g., the linker, the primer, or the polynucleotide) by chemical, enzymatic or photochemical means. In embodiments, the cleavable site includes one or more deoxyuracil nucleobases (dUTPs).

In embodiments, the plurality of oligonucleotides are covalently attached to the solid support (i.e., the solid support includes a plurality of immobilized oligonucleotides). In embodiments, the 5′ end of each oligonucleotide contains a reacted functional group that serves to tether the immobilized oligonucleotide to the solid support (e.g., a bioconjugate linker). Non-limiting examples of covalent attachment include amine-modified polynucleotides reacting with epoxy or isothiocyanate groups on the solid support, succinylated polynucleotides reacting with aminophenyl or aminopropyl functional groups on the solid support, dibenzocycloctyne-modified polynucleotides reacting with azide functional groups on the solid support (or vice versa), trans-cyclooctyne-modified polynucleotides reacting with tetrazine or methyl tetrazine groups on the solid support (or vice versa), disulfide modified polynucleotides reacting with mercapto-functional groups on the solid support, amine-functionalized polynucleotides reacting with carboxylic acid groups via 1-ethyl-3-(3-dimethylaminopropyl)-carbodiimide hydrochloride (EDC) chemistry, thiol-modified polynucleotides attaching to a solid support via a disulfide bond or maleimide linkage, alkyne-modified polynucleotides attaching to a solid support via copper-catalyzed click reactions to azide functional groups on the solid support, and acrydite-modified polynucleotides polymerizing with free acrylic acid monomers on the solid support to form polyacrylamide or reacting with thiol groups on the solid support. In embodiments, the primer is attached to the solid support polymer through electrostatic binding. For example, the negatively charged phosphate backbone of the primer may be bound electrostatically to positively charged monomers in the solid support.

In embodiments, each of the plurality of immobilized oligonucleotides (e.g., immobilized primers) is about 5 to about 25 nucleotides in length. In embodiments, each of the plurality of immobilized oligonucleotides (e.g., immobilized primers) is about 10 to about 40 nucleotides in length. In embodiments, each of the plurality of immobilized oligonucleotides (e.g., immobilized primers) is about 5 to about 100 nucleotides in length. In embodiments, each of the plurality of immobilized oligonucleotides (e.g., immobilized primers) is about 20 to 200 nucleotides in length. In embodiments, each of the plurality of immobilized oligonucleotides (e.g., immobilized primers) about or at least about 5, 6, 7, 8, 9, 10, 12, 15, 18, 20, 25, 30, 35, 40, 50 or more nucleotides in length.

In embodiments, the immobilized oligonucleotides include one or more phosphorothioate nucleotides. In embodiments, the immobilized oligonucleotides include a plurality of phosphorothioate nucleotides. In embodiments, about or at least about 20%, 30%, 40%, 50%, 60%, 70%, 80%, 90%, or about 100% of the nucleotides in the immobilized oligonucleotides are phosphorothioate nucleotides. In embodiments, most of the nucleotides in the immobilized oligonucleotides are phosphorothioate nucleotides. In embodiments, all of the nucleotides in the immobilized oligonucleotides are phosphorothioate nucleotides. In embodiments, none of the nucleotides in the immobilized oligonucleotides are phosphorothioate nucleotides. In embodiments, the 5′ end of the immobilized oligonucleotide includes one or more phosphorothioate nucleotides. In embodiments, the 5′ end of the immobilized oligonucleotide includes between one and five phosphorothioate nucleotides.

In embodiments, the template polynucleotide (e.g., the template nucleic acid) includes a first adapter and a second adapter, wherein the first adapter is a Y-adapter, a hairpin adapter, a blunt-ended adapter, or an adapter including a single-strand overhang and the second adapter is a Y-adapter, a hairpin adapter, a blunt-ended adapter, or an adapter including a single-strand overhang. In embodiments, the template nucleic acid includes a first adapter and a second adapter, wherein the first adapter is a Y-adapter and the second adapter is a Y-adapter. In embodiments, the template nucleic acid includes a first adapter and a second adapter, wherein the first adapter is a Y-adapter and the second adapter is a hairpin adapter. In embodiments, the template nucleic acid includes a first adapter and a second adapter, wherein the first adapter is a hairpin adapter and the second adapter is a Y-adapter. In embodiments, the template nucleic acid includes a first adapter and a second adapter, wherein the first adapter is a hairpin adapter and the second adapter is a hairpin adapter.

In some embodiments, the adapter is a Y-adapter. In embodiments, a Y-adapter includes a first strand and a second strand where a portion of the first strand (e.g., 3′-portion) is complementary, or substantially complementary, to a portion (e.g., 5′-portion) of the second strand. In embodiments, a Y-adapter includes a first strand and a second strand where a 3′-portion of the first strand is hybridized to a 5′-portion of the second strand. In embodiments, the 3′-portion of the first strand that is substantially complementary to the 5′-portion of the second strand forms a duplex including double stranded nucleic acid. Accordingly, a Y-adapter often includes a first end including a duplex region including a double stranded nucleic acid, and a second end including a forked region including a 5′-arm and a 3′-arm. In some embodiments, a 5′-portion of the first stand (e.g., 5′-arm) and a 3′-portion of the second strand (3′-arm) are not complementary. In embodiments, the first and second strands of a Y-adapter are not covalently attached to each other. In embodiments, the Y-adapter includes (i) a first strand having a 5′-arm and a 3′-portion, and (ii) a second strand having a 3′-arm and a 5′-portion, wherein the 3′-portion of the first strand is substantially complementary to the 5′-portion of the second strand, and the 5′-arm of the first strand is not substantially complementary to the 3′-arm of the second strand. In some embodiments, the first adapter includes a sample barcode sequence, a molecular identifier sequence, or both a sample barcode sequence and a molecular identifier sequence. In some embodiments, the first adapter includes a sample barcode sequence (e.g., a 6-10 nucleotide sequence).

In embodiments, ligating includes ligating both the 3′ end and the 5′ end of the duplex region of the first adapter to a double stranded nucleic acid. In embodiments, ligating includes ligating either the 3′ end or the 5′ end of the duplex region of the first adapter to a double stranded nucleic acid. In embodiments, ligating includes ligating the 5′ end of the duplex region of the first adapter to the double stranded nucleic acid and not the 3′ end of the duplex region. In embodiments, the method includes ligating a first adapter to a first end of the double stranded nucleic acid wherein both strands of the double stranded nucleic acid are ligated to the first adapter. In embodiments, the method includes ligating a first adapter to a first end of the double stranded nucleic acid wherein one strand of the double stranded nucleic acid is ligated to the first adapter.

In some embodiments, each strand of a Y-adapter, each of the non-complementary arms of a Y-adapter, or a duplex portion of a Y-adapter has a length independently selected from at least 5, at least 10, at least 15, at least 25, and at least 40 nucleotides. In some embodiments, each strand of a Y-adapter, each of the non-complementary arms of a Y-adapter, or a duplex portion of a Y-adapter has a length in a range independently selected from 15 to 500 nucleotides, 15-250 nucleotides, 15 to 200 nucleotides, 15 to 150 nucleotides, 20 to 100 nucleotides, 20 to 50 nucleotides and 10-50 nucleotides. In embodiments, one or both non-complementary arms of the Y-adapter is about or at least about 10, 15, 20, 25, 30, 35, 40, 45, 50, or more nucleotides in length. In embodiments, one or both non-complementary arms of the Y-adapter is about or at least about 20 nucleotides in length. In embodiments, one or both non-complementary arms of the Y-adapter is about or at least about 30 nucleotides in length. In embodiments, one or both non-complementary arms of the Y-adapter is about or at least about 40 nucleotides in length. In embodiments, the duplex portion of a Y-adapter is about or at least about 5, 10, 15, 20, 25, 30, or more nucleotides in length. In embodiments, the duplex portion of a Y-adapter is about 5-50, 5-25, or 10-15 nucleotides in length. In embodiments, the duplex portion of a Y-adapter is about or at least about 10 nucleotides in length. In embodiments, the duplex portion of a Y-adapter is about or at least about 15 nucleotides in length. In embodiments, the duplex portion of a Y-adapter is about or at least about 12 nucleotides in length. In embodiments, the duplex portion of a Y-adapter is about or at least about 20 nucleotides in length.

In some embodiments, a Y-adapter includes a first end including a duplex region including a double stranded nucleic acid, and a second end including a forked region, where the first end is configured for ligation to an end of a double stranded nucleic acid (e.g., a nucleic acid fragment, e.g., a library insert). In embodiments, a duplex end of a Y-adapter includes a 5′-overhang or a 3′-overhang that is complementary to a 3′-overhang or a 5′-overhang of an end of a double stranded nucleic acid. In some embodiments, a duplex end of a Y-adapter includes a blunt end that can be ligated to a blunt end of a double stranded nucleic acid. In certain embodiment, a duplex end of a Y-adapter includes a 5′-end that is phosphorylated.

In some embodiments, the first and/or second adapter (e.g., one or both strands of a Y-adapter) include one or more of a primer binding site, a capture nucleic acid binding site (e.g., a nucleic acid sequence complementary to a capture nucleic acid), a UMI, a sample barcode, a sequencing adapter, a label, a binding motif, the like or combinations thereof. In some embodiments, a non-complementary portion (e.g., 5′-arm and/or 3′-arm) of a Y-adapter includes one or more of a primer binding site, a capture nucleic acid binding site (e.g., a nucleic acid sequence complementary to a capture nucleic acid), a UMI, a sample barcode, a sequencing adapter, a label, a binding motif, the like or combinations thereof. In certain embodiments, a non-complementary portion of a Y-adapter includes a primer binding site. In certain embodiments, a non-complementary portion of a Y-adapter includes a binding site for a capture nucleic acid. In certain embodiments, a non-complementary portion of a Y-adapter includes a primer binding site and a UMI. In certain embodiments, a non-complementary portion of a Y-adapter includes a binding motif. In embodiments, the first and/or second adapter (e.g., one or both strands of a Y-adapter) does not include a UMI or sample barcode.

In embodiments, a complementary strand (e.g., a 3′-portion or 5′-portion) of a Y-adapter includes a primer binding site. In certain embodiments, a complementary strand (e.g., a 3′-portion or 5′-portion) of a Y-adapter includes a binding site for a capture nucleic acid. In certain embodiments, a complementary strand (e.g., a 3′-portion or 5′-portion) of a Y-adapter includes a primer binding site and a UMI. In certain embodiments, a complementary strand (e.g., a 3′-portion or 5′-portion) of a Y-adapter includes a binding motif.

In some embodiments, each of the non-complementary portions (i.e., arms) of a Y-adapter independently have a predicted, calculated, mean, average or absolute melting temperature (Tm) that is greater than 50° C., greater than 55° C., greater than 60° C., greater than 65° C., greater than 70° C. or greater than 75° C. In some embodiments, each of the non-complementary portions of a Y-adapter independently have a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm is about or at least about 70° C. In embodiments, the Tm is about or at least about 75° C. In embodiments, the Tm is about or at least about 80° C. In embodiments, the Tm is a calculated Tm. Tm's are routinely calculated by those skilled in the art, such as by commercial providers of custom oligonucleotides. In embodiments, the Tm for a given sequence is determined based on that sequence as an independent oligo. In embodiments, Tm is calculated using web-based algorithms, such as Primer3 and Primer3Plus (www.bioinformatics.nl/cgi-bin/primer3plus/primer3plus.cgi) using default parameters. The Tm of a non-complementary portion of a Y-adapter can be changed (e.g., increased) to a desired Tm using a suitable method, for example by changing (e.g., increasing) GC content, changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, each of the non-complementary portion of a Y-adapter independently includes one or more modified nucleotides, nucleotide analogues and/or modified nucleotides bonds.

In some embodiments, each of the non-complementary portions of a Y-adapter independently includes a GC content of greater than 40%, greater than 50%, greater than 55%, greater than 60% greater than 65% or greater than 70%. In certain embodiments, each of the non-complementary portions of a Y-adapter independently includes a GC content in a range of 40-100%, 50-100%, 60-100% or 70-100%. In embodiments, one or both non-complementary portions of a Y-adapter have a GC content of about or more than about 40%. In embodiments, one or both non-complementary portions of a Y-adapter have a GC content of about or more than about 50%. In embodiments, one or both non-complementary portions of a Y-adapter have a GC content of about or more than about 60%. Non-base modifiers can also be incorporated into a non-complementary portion of a Y-adapter to increase Tm, non-limiting examples of which include a minor grove binder (MGB), spermine, G-clamp, a Uaq anthraquinone cap, the like or combinations thereof.

In certain embodiments, a duplex region of a Y-adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of a duplex region of the Y-adapter is about or more than about 30° C. In embodiments, the Tm of a duplex region of the Y-adapter is about or more than about 35° C. In embodiments, the Tm of a duplex region of the Y-adapter is about or more than about 40° C. In embodiments, the Tm of a duplex region of the Y-adapter is about or more than about 45° C. In embodiments, the Tm of a duplex region of the Y-adapter is about or more than about 50° C.

In some embodiments, the adapter is hairpin adapter. In embodiments, a hairpin adapter includes a single nucleic acid strand including a stem-loop structure. A hairpin adapter can be any suitable length. In some embodiments, a hairpin adapter is at least 40, at least 50, or at least 100 nucleotides in length. In some embodiments, a hairpin adapter has a length in a range of 45 to 500 nucleotides, 75-500 nucleotides, 45 to 250 nucleotides, 60 to 250 nucleotides or 45 to 150 nucleotides. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, the second adapter includes a sample barcode sequence, a molecular identifier sequence, or both a sample barcode sequence and a molecular identifier sequence. In some embodiments, the second adapter includes a sample barcode sequence.

In some embodiments, a duplex region or stem portion of a hairpin adapter includes an end that is configured for ligation to an end of double stranded nucleic acid (e.g., a nucleic acid fragment, e.g., a library insert). In embodiments, an end of a duplex region or stem portion of a hairpin adapter includes a 5′-overhang or a 3′-overhang that is complementary to a 3′-overhang or a 5′-overhang of one end of a double stranded nucleic acid. In some embodiments, an end of a duplex region or stem portion of a hairpin adapter includes a blunt end that can be ligated to a blunt end of a double stranded nucleic acid. In certain embodiment, an end of a duplex region or stem portion of a hairpin adapter includes a 5′-end that is phosphorylated. In some embodiments, a stem portion of a hairpin adapter is at least 15, at least 25, or at least 40 nucleotides in length. In some embodiments, a stem portion of a hairpin adapter has a length in a range of 15 to 500 nucleotides, 15-250 nucleotides, 15 to 200 nucleotides, 15 to 150 nucleotides, 20 to 100 nucleotides or 20 to 50 nucleotides.

In embodiments, ligating includes ligating both the 3′ end and the 5′ end of the duplex region of the second adapter to the double stranded nucleic acid. In embodiments, ligating includes ligating either the 3′ end or the 5′ end of the duplex region of the second adapter to the double stranded nucleic acid. In embodiments, ligating includes ligating the 5′ end of the duplex region of the second adapter to the double stranded nucleic acid and not the 3′ end of the duplex region.

In some embodiments, the loop of a hairpin adapter includes one or more of the following: a primer binding site, a capture nucleic acid binding site (e.g., a nucleic acid sequence complementary to a capture nucleic acid), a UMI, a sample barcode, a sequencing adapter, a label, the like or combinations thereof. In certain embodiments, a loop of a hairpin adapter includes a primer binding site. In certain embodiments, a loop of a hairpin adapter includes a primer binding site and a UMI. In certain embodiments, a loop of a hairpin adapter includes a binding motif.

In some embodiments, the loop of a hairpin adapter has a predicted, calculated, mean, average or absolute melting temperature (Tm) that is greater than 50° C., greater than 55° C., greater than 60° C., greater than 65° C., greater than 70° C. or greater than 75° C. In some embodiments, a loop of a hairpin adapter has a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm of the loop is about 65° C. In embodiments, the Tm of the loop is about 75° C. In embodiments, the Tm of the loop is about 85° C. The Tm of a loop of a hairpin adapter can be changed (e.g., increased) to a desired Tm using a suitable method, for example by changing (e.g., increasing GC content), changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, a loop of a hairpin adapter includes one or more modified nucleotides, nucleotide analogues and/or modified nucleotides bonds.

In some embodiments, the loop of a hairpin adapter independently includes a GC content of greater than 40%, greater than 50%, greater than 55%, greater than 60% greater than 65% or greater than 70%. In certain embodiments, a loop of a hairpin adapter independently includes a GC content in a range of 40-100%, 50-100%, 60-100% or 70-100%. In embodiments, the loop has a GC content of about or more than about 40%. In embodiments, the loop has a GC content of about or more than about 50%. In embodiments, the loop has a GC content of about or more than about 60%. Non-base modifiers can also be incorporated into a loop of a hairpin adapter to increase Tm, non-limiting examples of which include a minor grove binder (MGB), spermine, G-clamp, a Uaq anthraquinone cap, the like or combinations thereof. A loop of a hairpin adapter can be any suitable length. In some embodiments, a loop of a hairpin adapter is at least 15, at least 25, or at least 40 nucleotides in length. In some embodiments, a hairpin adapter has a length in a range of 15 to 500 nucleotides, 15-250 nucleotides, 20 to 200 nucleotides, 30 to 150 nucleotides or 50 to 100 nucleotides.

In certain embodiments, a duplex region or stem region of a hairpin adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of the stem region is about or more than about 35° C. In embodiments, the Tm of the stem region is about or more than about 40° C. In embodiments, the Tm of the stem region is about or more than about 45° C. In embodiments, the Tm of the stem region is about or more than about 50° C.

In embodiments, the template polynucleotide (e.g., the template nucleic acid) is a double-stranded polynucleotide. In embodiments, the double-stranded polynucleotide includes genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA). In embodiments, the template polynucleotide includes genomic DNA. In embodiments, the template polynucleotide includes complementary DNA (cDNA). In embodiments, the template polynucleotide includes cell-free DNA (cfDNA). In embodiments, the template nucleic acid is a single-stranded polynucleotide.

In embodiments, the template polynucleotide (e.g., double-stranded polynucleotide) is about 100 to 1000 nucleotides in length. In embodiments, the template polynucleotide is about 350 nucleotides in length. In embodiments, the template polynucleotide is about 10, 20, 50, 100, 150, 200, 300, or 500 nucleotides in length. The template polynucleotide molecules can vary length, such as about 100-300 nucleotides long, about 300-500 nucleotides long, or about 500-1000 nucleotides long. In embodiments, the template polynucleotide molecule is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides. In embodiments, the template polynucleotide molecule is about 150 nucleotides. In embodiments, the template polynucleotide is about 100-1000 nucleotides long. In embodiments, the template polynucleotide is about 100-300 nucleotides long. In embodiments, the template polynucleotide is about 300-500 nucleotides long. In embodiments, the template polynucleotide is about 500-1000 nucleotides long. In embodiments, the template polynucleotide molecule is about 100 nucleotides. In embodiments, the template polynucleotide molecule is about 300 nucleotides. In embodiments, the template polynucleotide molecule is about 500 nucleotides. In embodiments, the template polynucleotide molecule is about 1000 nucleotides.

In embodiments the template polynucleotide (e.g., genomic template DNA) is first treated to form single-stranded linear nucleic acid fragments (e.g., ranging in length from about 50 to about 600 nucleotides). Treatment typically entails fragmentation, such as by chemical fragmentation, enzymatic fragmentation, or mechanical fragmentation, followed by denaturation to produce single-stranded DNA fragments. In embodiments, the template polynucleotide includes an adapter. The adapter may have other functional elements including tagging sequences (i.e., a barcode), attachment sequences, palindromic sequences, restriction sites, sequencing primer binding sites, functionalization sequences, and the like. Barcodes can be of any of a variety of lengths. In embodiments, the primer includes a barcode that is 10-50, 20-30, or 4-12 nucleotides in length. In embodiments, the adapter includes a primer binding sequence that is complementary to at least a portion of a primer (e.g., a sequencing primer binding sequence complementary to at least a portion of a sequencing primer). Primer binding sites can be of any suitable length. In embodiments, a primer binding site is about or at least about 10, 15, 20, 25, 30, or more nucleotides in length. In embodiments, a primer binding site is 10-50, 15-30, or 20-25 nucleotides in length. In embodiments the template polynucleotide is cfDNA.

In embodiments, the template polynucleotide includes known adapter sequences on the 5′ and 3′ ends, for example when the template polynucleotide is part of a library prepared for next-generation sequencing. An adapter may include a platform primer sequence (PP1 and PP2) such as the universal P5 and P7 sequences, a sequencing primer binding sequence (SP1 and SP2), and optionally one or two barcode/indexes (BC1 and BC2). As used herein, the terms “library”, “RNA library” or “DNA library” or “library of DNA molecules” are used in accordance with their plain ordinary meaning and refer to a collection or a population of similarly sized nucleic acid fragments with known adapter sequences (e.g., known adapters attached to the 5′ and 3′ ends of each of the fragments). In embodiments, the library includes a plurality of nucleic acid fragments including one or more adapter sequences. In embodiments, the library includes circular nucleic acid templates. Libraries are typically prepared from input RNA, DNA, or cDNA and are processed by fragmentation, size selection, end-repair, adapter ligation, amplification, and purification. Alternative amplification-free (i.e., PCR free) methods for preparing a library of molecules include shearing input polynucleotides, size selecting and ligating adapters. A library may correspond to a single sample or a single origin. Multiple libraries, each with their own unique adapter sequences, may be pooled and sequenced in the same sequencing run using the methods described herein. Examples of platform primers include P7 and P5 primers, or S1 and S2 sequences, or the reverse complements thereof. A “platform primer binding sequence” refers to a sequence or portion of an oligonucleotide that is capable of binding to a platform primer (e.g., the platform primer binding sequence is complementary to the platform primer). In embodiments, a platform primer binding sequence may form part of an adapter. In embodiments, a platform primer binding sequence is complementary to a platform primer sequence. In embodiments, a platform primer binding sequence is complementary to a primer. The P5 and P7 primers are used on the surface of commercial flow cells for sequencing on various Illumina platforms. The P5 and P7 adapter sequences are described in U.S. Patent Publication No. 2011/0059865 A1, which is incorporated herein by reference in its entirety. The terms P5 and P7 may be used when referring to amplification primers, e.g., universal primers. The terms P5′ (P5 prime) and P7′ (P7 prime) refer to the complement of P5 and P7, respectively. In embodiments, adapters include sequences that bind to sequencing primers. In embodiments, adapters include sequences that bind to immobilized oligonucleotides (e.g., P7 and P5 sequences, or S1 and S2 sequences) or reverse complements thereof.

In embodiments, removing the template polynucleotide includes denaturing the template polynucleotide. Template polynucleotide denaturation may be performed in solutions with high pH and/or organic solutions capable of denaturing DNA. In some embodiments, the template polynucleotide may be removed via heat denaturation. In embodiments, removing the template polynucleotide includes contacting the template polynucleotide with a denaturant, wherein the denaturant is a buffered solution including betaine, dimethyl sulfoxide (DMSO), ethylene glycol, formamide, glycerol, guanidine thiocyanate, 4-methylmorpholine 4-oxide (NMO), or a mixture thereof. In some embodiments, denaturation is achieved by exposure to chemical denaturants such as urea or formamide, with concentrations suitably adjusted, or using high or low pH (e.g., pH between 4-6 or 8-9). In embodiments, the denaturant is a buffered solution including betaine, dimethyl sulfoxide (DMSO), ethylene glycol, formamide, glycerol, guanidine thiocyanate, 4-methylmorpholine 4-oxide (NMO), or a mixture thereof. In embodiments, the first denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof.

In embodiments, forming the plurality of immobilized amplification products includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, generating a double-stranded amplification product includes a bridge polymerase chain reaction (bPCR) amplification. In embodiments, generating a double-stranded amplification product includes a thermal bridge polymerase chain reaction (t-bPCR) amplification. In embodiments, generating a double-stranded amplification product includes a chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions.

In embodiments, forming a plurality of amplification products includes bridge amplification; for example, as exemplified by the disclosures of U.S. Pat. Nos. 5,641,658; 7,115,400; 7,790,418; U.S. Patent Publ. No. 2008/0009420, each of which is incorporated herein by reference in its entirety. In general, bridge amplification uses repeated steps of annealing of primers to templates, primer extension, and separation of extended primers from templates. Because the forward and reverse primers are attached to the solid support, the extension products released upon separation from an initial template are also attached to the solid support. Both strands are immobilized on the solid support at the 5′ end, preferably via a covalent attachment. The 3′ end of an amplification product is then permitted to anneal to a nearby reverse primer, forming a “bridge” structure. The reverse primer is then extended to produce a further template molecule that can form another bridge. During bridge PCR, additional chemical additives may be included in the reaction mixture, in which the DNA strands are denatured by flowing a denaturant over the DNA, which chemically denatures complementary strands. This is followed by washing out the denaturant and reintroducing a polymerase in buffer conditions that allow primer annealing and extension.

In embodiments, forming a plurality of amplification products includes amplifying the template polynucleotide or complement thereof on a solid support including a plurality of primers attached to the solid support, wherein the plurality of primers include a plurality of forward primers with complementarity to the template polynucleotide and a plurality of reverse primers with complementarity to a complement of the template polynucleotide, and the amplifying includes a plurality of cycles of strand denaturation, primer hybridization, and primer extension.

In embodiments, the plurality of strand denaturation cycles may be different for one or more cycles, wherein the initial denaturation cycle is maintained at different conditions from the remaining denaturation cycles. For example, in embodiments, the initial denaturation cycle is at about 85° C.-95° C. for about 1 minute to about 10 minutes, whereas denaturation in the remaining cycles is different (e.g., about 85° C. for about 15-30 sec). In embodiments, the initial denaturation is maintained at about 85° C.-95° C. for about 5 minutes to about 10 minutes. In embodiments, the initial denaturation is maintained at 90° C.-95° C. for about 1 to 10 minutes. In embodiments, the initial denaturation is maintained at 80° C.-85° C. for about 1 to 10 minutes. In embodiments, the initial denaturation is maintained at 85° C.-90° C. for about 1 to 10 minutes. In embodiments, the initial denaturation is maintained at about 85° C.-95° C. for about 1 minutes to about 10 minutes. In embodiments, the initial denaturation is maintained at about 95° C. for about 5 minutes to about 10 minutes. In embodiments, the initial denaturation is maintained at about 85° C.-95° C. for about 5 minutes to about 10 minutes.

In embodiments, forming a plurality of amplification products includes a thermal bridge polymerase chain reaction (t-bPCR) amplification. In embodiments, the plurality of cycles includes thermally cycling between (i) about 85° C. for about 15-30 sec for denaturation, and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer.

In embodiments, forming a plurality of amplification products includes chemical bridge polymerase chain reaction (c-bPCR) amplification. In embodiments, forming a plurality of amplification products includes denaturation using a chemical denaturant. In embodiments, forming a plurality of amplification products includes denaturation using acetic acid, hydrochloric acid, nitric acid, formamide, guanidine, sodium salicylate, sodium hydroxide, dimethyl sulfoxide (DMSO), propylene glycol, urea, or a mixture thereof. In embodiments, the chemical denaturant is sodium hydroxide or formamide. In embodiments, forming a plurality of amplification products includes thermal bridge polymerase chain reaction (t-bPCR) amplification. In embodiments, forming a plurality of amplification products includes chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a significantly lower concentration than traditional chemical bridge polymerase chain reactions.

In embodiments, forming a plurality of amplification products includes fluidic cycling between an extension mixture that includes a polymerase and dNTPs, and a chemical denaturant. In embodiments, the polymerase is a strand-displacing polymerase or a non-strand displacing polymerase. In embodiments, the solutions are thermally cycled between about 40° C. to about 65° C. during fluidic cycling of the extension mixture and the chemical denaturant. For example, the extension cycle is maintained at a temperature of 55° C.-65° C., followed by a denaturation cycle that is maintained at a temperature of 40° C.-65° C., or by a denaturation step in which the temperature starts at 60° C.-65° C. and is ramped down to 40° C. prior to exchanging the reagent. In embodiments, step (b) includes modulating the reaction temperature prior to initiating the next cycle. In embodiments, the denaturation cycle and/or the extension cycle is maintained at a temperature for a sufficient amount of time, and prior to starting the next cycle the temperature is modulated (e.g., increased relative to the starting temperature or reduced relative to the starting temperature). In embodiments, the denaturation cycle is performed at a temperature of 60° C.-65° C. for about 5-45 sec, then the temperature is reduced (e.g., lowered to about 40° C.) before starting an extension cycle (i.e., before introducing an extension mixture). Lowering the temperature, even in the presence of a chemical denaturant, facilitates primer hybridization in the subsequent step when the amplicons are exposed to conditions that promote hybridization. In embodiments, the extension cycle is performed at a temperature of 50° C.-60° C. for about 0.5-2 minutes, then the temperature is increased (e.g., raised to between about 60° C. to about 70° C., or to about 65° C. to about 72° C.) after introducing the extension mixture. In embodiments, the cycling between the extension mixture and the chemical denaturant is performed at least 5, at least 10, at least 20, at least 30, at least 40, at least 50, at least 75, at least 100, or at least 200 times. In embodiments, the cycling between the extension mixture and the chemical denaturant is performed about 5, about 10, about 20, about 30, about 40, about 50, about 75, about 100, or about 200 times. In embodiments, the cycling between the extension mixture and the chemical denaturant is performed a total of 5, 10, 20, 30, 40, 50, 75, 100, 200, or more times. In embodiments, the fluidic cycling is performed in the presence of about 2 to about 15 mM Mg²⁺. In embodiments, the fluidic cycling is performed in the presence of about 2, about 3, about 4, about 5, about 6, about 7, about 8, about 9, about 10, about 11, about 12, about 13, about 14, or about 15 mM Mg²⁺.

In embodiments, forming a plurality of amplification products includes a plurality of strand denaturation cycles, wherein the initial denaturation cycle is at different conditions from the remaining denaturation cycles. For example, in embodiments, the initial denaturation cycle is at about 85° C.-95° C. for about 1 minute to about 10 minutes, whereas denaturation in the remaining cycles is different (e.g. about 85° C. for about 15-30 sec). In embodiments, forming a plurality of amplification products includes an initial denaturation at about 85° C.-95° C. for about 5 minutes to about 10 minutes. In embodiments, forming a plurality of amplification products includes an initial denaturation at 90° C.-95° C. for about 1 to 10 minutes. In embodiments, forming a plurality of amplification products includes an initial denaturation at 80° C.-85° C. for about 1 to 10 minutes. In embodiments, forming a plurality of amplification products includes an initial denaturation at 85° C.-90° C. for about 1 to 10 minutes. In embodiments, amplification is performed according to a method as described in U.S. Patent Pub. 2022/0235410, which is incorporated herein by reference in its entirety.

In embodiments, the plurality of cycles includes thermally cycling between (i) about 80° C. to 90° C. for denaturation, and (ii) about 55° C. to about 65° C. for annealing/extension of the primer. In embodiments, the plurality of cycles includes thermally cycling between (i) about 85° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer. In embodiments, the plurality of cycles includes thermally cycling between (i) less than 80° C. (e.g., 70 to 80° C.) for denaturation, and (ii) about 55° C. to about 65° C. for annealing/extension of the primer. In embodiments, the plurality of cycles includes thermally cycling between (i) about 70° C. for denaturation, and (ii) about 65° C. for annealing/extension of the primer. In embodiments, the plurality of cycles includes thermally cycling between (i) about 75° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer.

In embodiments, the plurality of cycles includes thermally cycling between (i) about 85° C. for less than 1 minute for denaturation, and (ii) about 65° C. for about 1 to 2 minutes for annealing/extension of the primer. In embodiments, the plurality of cycles includes thermally cycling between (i) about 85° C. for less than 1 minute for denaturation, and (ii) about 60° C. to about 65° C. for about 1 minute for annealing/extension of the primer. In embodiments, the plurality of cycles includes thermally cycling between (i) about 85° C. for about 15-30 sec for denaturation and (ii) about 65° C. for about 1 minute for annealing/extension of the primer. In embodiments, the plurality of cycles includes thermally cycling between (i) about 85° C. for about 30 sec for denaturation and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer. In embodiments, the plurality of cycles includes thermally cycling between (i) about 85° C. for about 15-30 sec for denaturation, and (ii) about 65° C. for about 1 minute for annealing/extension of the primer.

In embodiments, the plurality of denaturation steps is at a temperature of about 80° C.-95° C. In embodiments, the plurality of denaturation steps is at a temperature of about 80° C.-90° C. In embodiments, the plurality of denaturation steps is at a temperature of about 85° C.-90° C. In embodiments, the plurality of denaturation steps is at a temperature of about 81° C., 82° C., 83° C., 84° C., 85° C., 86° C., 87° C., 88° C., 89° C., or about 90° C. In embodiments, the plurality of denaturation steps is at a temperature of about 70° C.-85° C. In embodiments, the plurality of denaturation steps is at a temperature of about 70° C.-80° C. In embodiments, the plurality of denaturation steps is at a temperature of about 75° C.-80° C. In embodiments, the plurality of denaturation steps is at a temperature of about 70° C., 71° C., 72° C., 73° C., 74° C., 75° C., 76° C., 77° C., 78° C., 79° C., or about 80° C. In embodiments, the annealing/extension of the primer cycle is at a temperature of about 55° C., 56° C., 57° C., 58° C., 59° C., 60° C., 61° C., 62° C., 63° C., 64° C., or about 65° C.

In embodiments, the plurality of cycles includes thermally cycling between (i) about 80° C. to 90° C. for denaturation, and (ii) about 55° C. to about 65° C. for annealing/extension of the primer. In embodiments, the plurality of cycles includes thermally cycling between (i) about 85° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer. In embodiments, the plurality of cycles includes thermally cycling between (i) less than 80° C. (e.g., 70 to 80° C.) for denaturation, and (ii) about 55° C. to about 65° C. for annealing/extension of the primer. In embodiments, the plurality of cycles includes thermally cycling between (i) about 70° C. for denaturation, and (ii) about 65° C. for annealing/extension of the primer. In embodiments, the plurality of cycles includes thermally cycling between (i) about 75° C. for denaturation, and (ii) about 55° C. for annealing/extension of the primer. In embodiments, the plurality of cycles includes thermally cycling between (i) about 85° C. for denaturation, and (ii) about 65° C. for annealing/extension of the primer.

In embodiments, the plurality of cycles includes thermally cycling between (i) about 85° C. for less than 1 minute for denaturation, and (ii) about 65° C. for about 1 to 2 minutes for annealing/extension of the primer. In embodiments, the plurality of cycles includes thermally cycling between (i) about 85° C. for less than 1 minute for denaturation, and (ii) about 60° C. to about 65° C. for about 1 minute for annealing/extension of the primer. In embodiments, the plurality of cycles includes thermally cycling between (i) about 85° C. for about 15-30 sec for denaturation and (ii) about 65° C. for about 1 minute for annealing/extension of the primer. In embodiments, the plurality of cycles includes thermally cycling between (i) about 85° C. for about 30 sec for denaturation and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer. In embodiments, the plurality of cycles includes thermally cycling between (i) about 85° C. for about 15-30 sec for denaturation, and (ii) about 65° C. for about 1 minute for annealing/extension of the primer. In embodiments, the temperature and duration for the annealing of the primer and the extension of the primer are different. In embodiments, the plurality of cycles includes thermally cycling between (i) about 90° C. to 95° C. for about 15 to 30 sec for denaturation and (ii) about 55° C. to about 65° C. for about 30 to 60 seconds for annealing and about 65° C. to 70° C. for about 30 to 60 seconds for extension of the primer. In embodiments, the plurality of denaturation steps is at a temperature of about 80° C.-95° C. In embodiments, the plurality of denaturation steps is at a temperature of about 80° C.-90° C. In embodiments, the plurality of denaturation steps is at a temperature of about 85° C.-90° C. In embodiments, the plurality of denaturation steps is at a temperature of about 81° C., 82° C., 83° C., 84° C., 85° C., 86° C., 87° C., 88° C., 89° C., or about 90° C. In embodiments, the plurality of denaturation steps is at a temperature of about 91° C., 92° C., 93° C., 94° C., 95° C., 96° C., 97° C., 98° C., or about 99° C. In embodiments, the plurality of denaturation steps is at a temperature of about 87° C., 88° C., 89° C., 90° C., 91° C., 92° C., 93° C., 94° C., or about 95° C. In embodiments, the plurality of denaturation steps is at a temperature of about 90° C., 91° C., 92° C., 93° C., 94° C., or about 95° C. In embodiments, the plurality of denaturation steps is at a temperature of about 70° C.-85° C. In embodiments, the plurality of denaturation steps is at a temperature of about 70° C.-80° C. In embodiments, the plurality of denaturation steps is at a temperature of about 75° C.-80° C. In embodiments, the plurality of denaturation steps is at a temperature of about 70° C., 71° C., 72° C., 73° C., 74° C., 75° C., 76° C., 77° C., 78° C., 79° C., or about 80° C. In embodiments, the annealing/extension of the primer cycle is at a temperature of about 55° C., 56° C., 57° C., 58° C., 59° C., 60° C., 61° C., 62° C., 63° C., 64° C., or about 65° C.

In embodiments, forming a plurality of amplification products includes incubation in a denaturant. In embodiments, the denaturant is acetic acid, ethylene glycol, hydrochloric acid, nitric acid, formamide, guanidine, sodium salicylate, sodium hydroxide, dimethyl sulfoxide (DMSO), propylene glycol, urea, or a mixture thereof. In embodiments, the denaturant is an additive that lowers a DNA denaturation temperature. In embodiments, the denaturant is betaine, dimethyl sulfoxide (DMSO), ethylene glycol, formamide, glycerol, guanidine thiocyanate, 4-methylmorpholine 4-oxide (NMO), or a mixture thereof. In embodiments, the denaturant is betaine, dimethyl sulfoxide (DMSO), ethylene glycol, formamide, glycerol, guanidine thiocyanate, or 4-methylmorpholine 4-oxide (NMO).

In embodiments, forming a plurality of amplification products includes a plurality of cycles of strand denaturation, primer hybridization, and primer extension. Although each cycle will include each of these three events (denaturation, hybridization, and extension), events within a cycle may or may not be discrete. For example, each step may have different reagents and/or reaction conditions (e.g., temperatures). Alternatively, some steps may proceed without a change in reaction conditions. For example, extension may proceed under the same conditions (e.g., same temperature) as hybridization. After extension, the conditions are changed to start a new cycle with a new denaturation step, thereby amplifying the amplicons. Primer extension products from an earlier cycle may serve as templates for a later amplification cycle. In embodiments, the plurality of cycles is about 5 to about 50 cycles. In embodiments, the plurality of cycles is about 10 to about 45 cycles. In embodiments, the plurality of cycles is about 10 to about 20 cycles. In embodiments, the plurality of cycles is about 20 to about 30 cycles. In embodiments, the plurality of cycles is 10 to 45 cycles. In embodiments, the plurality of cycles is 10 to 20 cycles. In embodiments, the plurality of cycles is 20 to 30 cycles. In embodiments, the plurality of cycles is about 10 to about 45 cycles. In embodiments, the plurality of cycles is about 20 to about 30 cycles.

In embodiments, forming a plurality of amplification products includes rolling circle amplification (RCA) (see, e.g., Lizardi et al., Nat. Genet. 19:225-232 (1998), which is incorporated herein by reference in its entirety). Several suitable RCA methods are known in the art. For example, RCA amplifies a circular polynucleotide (e.g., DNA) by polymerase extension of an amplification primer complementary to a portion of the template nucleic acid. This process generates copies of the circular polynucleotide template such that multiple complements of the template sequence arranged end to end in tandem are generated (i.e., a concatemer).

In embodiments, forming a plurality of amplification products includes exponential rolling circle amplification (eRCA). Exponential RCA is similar to the linear process except that it uses a second primer having a sequence that is identical to at least a portion of the circular template (Lizardi et al. Nat. Genet. 19:225 (1998)). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (Nilsson et al. Science 265(5181):208 5(1994)).

In embodiments, forming a plurality of amplification products includes hyperbranched rolling circle amplification (HRCA). Hyperbranched RCA uses a second primer complementary to the first amplification product. This allows products to be replicated by a strand-displacement mechanism, which can yield a drastic amplification within an isothermal reaction (Lage et al., Genome Research 13:294-307 (2003), which is incorporated herein by reference in its entirety).

In embodiments, the method includes amplifying a template nucleic acid (e.g., a template polynucleotide) by extending an amplification primer with a strand-displacing polymerase for about 10 seconds to about 30 minutes. In embodiments, the method includes amplifying a template nucleic acid by extending an amplification primer with a strand-displacing polymerase for about 30 seconds to about 16 minutes. In embodiments, the method includes amplifying a template nucleic acid by extending an amplification primer with a strand-displacing polymerase for about 30 seconds to about 10 minutes. In embodiments, the method includes amplifying a template nucleic acid by extending an amplification primer with a strand-displacing polymerase for about 30 seconds to about 5 minutes. In embodiments, the method includes amplifying a template nucleic acid by extending an amplification primer with a strand-displacing polymerase for about 1 second to about 5 minutes. In embodiments, the method includes amplifying a template nucleic acid by extending an amplification primer with a strand-displacing polymerase for about 1 second to about 2 minutes.

In embodiments, the method includes amplifying a template nucleic acid by extending an amplification primer with a strand-displacing polymerase at a temperature of about 20° C. to about 50° C. In embodiments, the method includes amplifying a template nucleic acid by extending an amplification primer with a strand-displacing polymerase at a temperature of about 30° C. to about 50° C. In embodiments, the method includes amplifying a template nucleic acid by extending an amplification primer with a strand-displacing polymerase at a temperature of about 25° C. to about 45° C. In embodiments, the method includes amplifying a template nucleic acid by extending an amplification primer with a strand-displacing polymerase at a temperature of about 35° C. to about 45° C. In embodiments, the method includes amplifying a template nucleic acid by extending an amplification primer with a strand-displacing polymerase at a temperature of about 35° C. to about 42° C. In embodiments, the method includes amplifying a template nucleic acid by extending an amplification primer with a strand-displacing polymerase at a temperature of about 37° C. to about 40° C.

In embodiments, the strand-displacing enzyme is an SD polymerase, Bst large fragment polymerase, or a phi29 polymerase or mutant thereof. In embodiments, the strand-displacing polymerase is phi29 polymerase, phi29 mutant polymerase or a thermostable phi29 mutant polymerase. A “phi polymerase” (or “Φ29 polymerase”) is a DNA polymerase from the Φ29 phage or from one of the related phages that, like Φ29, contain a terminal protein used in the initiation of DNA replication. For example, phi29 polymerases include the B103, GA-1, PZA, Φ15, BS32, M2Y (also known as M2), Nf, G1, Cp-1, PRD1, PZE, SFS, Cp-5, Cp-7, PR4, PR5, PR722, L17, Φ21, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase read length, enhance accuracy, increase phototolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes. For example, a thermostable phi29 mutant polymerase refers to an isolated bacteriophage phi29 DNA polymerase including at least one mutation selected from the group consisting of M8R, V51A, M97T, L123S, G197D, K209E, E221K, E239G, Q497P, K512E, E515A, and F526 (relative to wild type phi29 polymerase).

In embodiments, the double-stranded amplification product is provided in a clustered array. In embodiments, the clustered array includes a plurality of double-stranded amplification products localized to discrete sites on a solid support. In embodiments, the solid support is a bead. In embodiments, the solid support is substantially planar. In embodiments, the solid support is contained within a flow cell.

In embodiments, the amplification primers are each attached to the solid support (i.e., immobilized on the surface of a solid support). The polynucleotide molecules can be fixed to surface by a variety of techniques, including covalent attachment and non-covalent attachment. In embodiments, the polynucleotides are confined to an area of a discrete region (referred to as a cluster). The discrete regions may have defined locations in a regular array, which may correspond to a rectilinear pattern, circular pattern, hexagonal pattern, or the like. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas (e.g., overlapping clusters) of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. Interstitial regions will typically have a surface material that differs from the surface material of the features on the surface. For example, features of an array can have polynucleotides that exceeds the amount or concentration present at the interstitial regions. In some embodiments the polynucleotides and/or primers may not be present at the interstitial regions. In embodiments, at least two different primers are attached to the solid support (e.g., a forward and a reverse primer), which facilitates generating multiple amplification products from the first extension product or a complement thereof.

In embodiments, the clusters (e.g., overlapping clusters) have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 μm or a number or a range between any two of these values. In embodiments, the mean or median separation is about 0.1-10 microns. In embodiments, the mean or median separation is about 0.25-5 microns. In embodiments, the mean or median separation is about 0.5-2 microns. In embodiments, the mean or median separation is about or at least about 0.1 μm. In embodiments, the mean or median separation is about or at least about 0.25 μm. In embodiments, the mean or median separation is about or at least about 0.5 μm. In embodiments, the mean or median separation is about or at least about 1.0 μm. In embodiments, the mean or median separation is about or at least about 1.5 μm. In embodiments, the mean or median separation is about or at least about 2.0 μm. In embodiments, the mean or median separation is about or at least about 5.0 μm. In embodiments, the mean or median separation is about or at least about 10 μm. The mean or median separation may be measured center-to-center (i.e., the center of one cluster to the center of a second cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm. In embodiments, the mean or median separation is about or at least about 1.0, 1.1, 1.2, 1.3, 1.4, 1.5, 1.6, 1.7, 1.8, 1.9, or 2.0 μm. In embodiments, the mean or median separation is about 1.0, 1.1, 1.2, 1.3, 1.4, 1.5, 1.6, 1.7, 1.8, 1.9, or 2.0 μm.

In embodiments, the method further includes sequencing the plurality of amplification products. In embodiments, the method further includes sequencing one or more immobilized products. In embodiments, sequencing includes sequencing by synthesis, sequencing by binding, sequencing by ligation, or pyrosequencing. In embodiments, sequencing includes sequencing by synthesis. In embodiments, generating a first sequencing read or a second sequencing read includes a sequencing by synthesis process. In embodiments, generating a first sequencing read or a second sequencing read includes sequencing-by-binding. As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

In embodiments, the method further includes generating a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated. In embodiments, the method further includes incorporating one or more unmodified dNTPs or one or more ddNTPs into the 3′ end of the extended sequencing primer.

In embodiments, generating a sequencing read includes sequencing by synthesis, sequencing-by-binding, sequencing by ligation, or pyrosequencing.

In embodiments, sequencing includes a plurality of sequencing cycles. In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes 50 to 100 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue.

In embodiments, the method includes sequencing the first and/or the second strand of a double-stranded amplification product by extending a sequencing primer hybridized thereto (e.g., extending a sequencing primer hybridized to a sequencing primer binding sequence). A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

In embodiments, the methods of sequencing provided herein include aligning a portion of each sequencing read to a reference sequence. General methods for performing sequence alignments are known to those skilled in the art. Examples of suitable alignment algorithms, include but are not limited to the Needleman-Wunsch algorithm (see e.g. the EMBOSS Needle aligner available at www.ebi.ac.uk/Tools/psa/emboss_needle/, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/, optionally with default settings). Optimal alignment may be assessed using any suitable parameters of a chosen algorithm, including default parameters. In embodiments, the reference sequence is a reference genome. In embodiments, the methods of sequencing a template nucleic acid further include generating overlapping sequence reads and assembling them into a contiguous nucleotide sequence of a nucleic acid of interest. Assembly algorithms known in the art can align and merge overlapping sequence reads generated by methods of several embodiments herein to provide a contiguous sequence of a nucleic acid of interest. A person of ordinary skill in the art will understand which sequence assembly algorithms or sequence assemblers are suitable for a particular purpose taking into account the type and complexity of the nucleic acid of interest to be sequenced (e.g. genomic, PCR product, or plasmid), the number and/or length of deletion products or other overlapping regions generated, the type of sequencing methodology performed, the read lengths generated, whether assembly is de novo assembly of a previously unknown sequence or mapping assembly against a backbone sequence, etc. Furthermore, an appropriate data analysis tool will be selected based on the function desired, such as alignment of sequence reads, base-calling and/or polymorphism detection, de novo assembly, assembly from paired or unpaired reads, and genome browsing and annotation. In several embodiments, overlapping sequence reads can be assembled by sequence assemblers, including but not limited to ABySS, AMOS, Arachne WGA, CAP3, PCAP, Celera WGA Assembler/CABOG, CLC Genomics Workbench, CodonCode Aligner, Euler, Euler-sr, Forge, Geneious, MIRA, miraEST, NextGENe, Newbler, Phrap, TIGR Assembler, Sequencher, SeqMan NGen, SHARCGS, SSAKE, Staden gap4 package, VCAKE, Phusion assembler, Quality Value Guided SRA (QSRA), Velvet (algorithm), and the like. It will be understood that overlapping sequence reads can also be assembled into contigs or the full contiguous sequence of the nucleic acid of interest by available means of sequence alignment, computationally or manually, whether by pairwise alignment or multiple sequence alignment of overlapping sequence reads. Algorithms suited for short-read sequence data may be used in a variety of embodiments, including but not limited to Cross_match, ELAND, Exonerate, MAQ, Mosaik, RMAP, SHRiMP, SOAP, SSAHA2, SXOligoSearch, ALLPATHS, Edena, Euler-SR, SHARCGS, SHRAP, SSAKE, VCAKE, Velvet, PyroBayes, PbShort, and ssahaSNP. In embodiments, aligning to a reference sequence is useful to validate the approaches described herein.

A variety of suitable sequencing platforms are available for implementing methods disclosed herein (e.g., for performing the sequencing reaction). Non-limiting examples include SMRT (single-molecule real-time sequencing), ion semiconductor, pyrosequencing, sequencing by synthesis, sequencing by binding, combinatorial probe anchor synthesis, SOLiD sequencing (sequencing by ligation), and nanopore sequencing. Sequencing platforms include those provided by Singular Genomics™ (e.g., the G4™ system), Illumina™, Inc. (e.g., HiSeq™ MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). See, for example U.S. Pat. Nos. 7,211,390; 7,244,559; 7,264,929; 6,255,475; 6,013,445; 8,882,980; 6,664,079; and 9,416,409.

In embodiments, the methods of sequencing described herein further include computationally reconstructing sequences of a plurality of individual strands of original sample polynucleotides by removing barcode sequences and joining sequences for adjacent portions of the sample polynucleotide. Reconstruction can be performed on individual reads, or on consensus sequences produced from those reads.

In embodiments, the methods of sequencing described herein further include aligning computationally reconstructed sequences.

Flow cells provide a convenient format for housing an array of clusters produced by the methods described herein, in particular when subjected to an SBS or other detection technique that involves repeated delivery of reagents in cycles. For example, to initiate a first SBS cycle, one or more labeled nucleotides and a DNA polymerase in a buffer, can be flowed into/through a flow cell that houses an array of clusters. The clusters of an array where primer extension causes a labeled nucleotide to be incorporated can then be detected. Optionally, the nucleotides can further include a reversible termination moiety that temporarily halts further primer extension once a nucleotide has been added to a primer. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by the methods of the present disclosure are described, for example, in Bentley et al., Nature 456:53-59 (2008), US Patent Publication 2018/0274024, WO 2017/205336, US Patent Publication 2018/0258472, each of which are incorporated herein in their entirety for all purposes.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, sequencing is performed according to a “sequencing-by-binding” method (see, e.g., U.S. Pat. Pubs. US2017/0022553 and US2019/0048404, each of which is incorporated herein by reference in its entirety), which refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides. In embodiments, following the generation of a sequencing read, subsequent extension is performed using a plurality of standard (e.g., non-modified) dNTPs until the complementary strand is copied. In other embodiments, following the generation of a sequencing read, subsequent extension is performed using a plurality of dideoxy nucleotide triphosphates (ddNTPs) to prevent further extension of the first sequencing read product during a second sequencing read. In embodiments, following the identification of at least 5-10 (e.g., 11 to 200 nucleotides, or up to 1000 nucleotides), subsequent extension is performed using a plurality of standard (e.g., non-modified) dNTPs until the complementary strand is copied. In embodiments, following the identification of at least 5-10 (e.g., 11 to 200 nucleotides, or up to 1000 nucleotides), subsequent extension is performed using a plurality of dideoxy nucleotide triphosphates (ddNTPs) to prevent further extension of the sequencing read product.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. These such reactions can be done in a single experiment if each of the modified nucleotides has attached a different label, known to correspond to the particular nucleobase, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

In embodiments, the methods of sequencing a nucleic acid include extending a complementary polynucleotide (e.g., a primer) that is hybridized to the nucleic acid by incorporating a first nucleotide. In embodiments, the method includes a buffer exchange or wash step. In embodiments, the methods of sequencing a nucleic acid include a sequencing solution. The sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof; and (d) a guanine nucleotide, or analog thereof.

### III. Compositions & Kits

In an aspect is provided a kit. Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, e.g., deoxyribonucleotides, ribonucleotides, labeled nucleotides, and/or modified nucleotides), buffers, salts, and/or labels (e.g., fluorophores). In embodiments, the kit includes one or more reagents and one or more compositions useful for performing the methods as described herein.

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. The kit may also include a flow cell. In embodiments, kit includes the solid support and a flow cell carrier (e.g., a flow cell carrier as described in US 2021/0190668, which is incorporated herein by reference for all purposes).

In embodiments, the kit includes components useful for ligating polynucleotides using a ligation enzyme (e.g., CircLigase™ enzyme, Taq DNA Ligase, HiFi Taq DNA Ligase, T4 DNA ligase, T4 RNA ligase, T4 RNA ligase 2, or Ampligase® DNA Ligase). For example, such a kit further includes the following components: (a) reaction buffer for controlling pH and providing an optimized salt composition for a ligation enzyme (e.g., CircLigase™ enzyme, Taq DNA Ligase, HiFi Taq DNA Ligase, T4 DNA ligase, T4 RNA ligase 2, or Ampligase® DNA Ligase), and (b) ligation enzyme cofactors, such as ATP and a divalent ion (e.g., Mn²⁺ or Mg²⁺).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. As used herein, the term “fragmented kit” refers to a delivery system comprising two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits. In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

In embodiments, the template nucleic acid is at least 1000 bases (1 kb), at least 2 kb, at least 4 kb, at least 6 kb, at least 10 kb, at least 20 kb, at least 30 kb, at least 40 kb, or at least 50 kb in length. In embodiments, the entire sequence of the template nucleic acid is about 1 to 3 kb, and only a portion of that the template nucleic acid (e.g., 50 to 100 nucleotides) is sequenced at a time. In embodiments, the template nucleic acid is about 2 to 3 kb. In embodiments, the template nucleic acid is about 1 to 10 kb. In embodiments, the template nucleic acid is about 3 to 10 kb. In embodiments, the template nucleic acid is about 5 to 10 kb. In embodiments, the template nucleic acid is about 1 to 3 kb. In embodiments, the template nucleic acid is about 1 to 2 kb. In embodiments, the template nucleic acid is greater than 1 kb. In embodiments, the template nucleic acid is greater than 500 bases. In embodiments, the template nucleic acid is about 1 kb. In embodiments, the template nucleic acid is about 2 kb. In embodiments, the template nucleic acid is less than 1 kb. In embodiments, the template nucleic acid is about 500 nucleotides. In embodiments, the template nucleic acid is about 510 nucleotides. In embodiments, the template nucleic acid is about 520 nucleotides. In embodiments, the template nucleic acid is about 530 nucleotides. In embodiments, the template nucleic acid is about 540 nucleotides. In embodiments, the template nucleic acid is about 550 nucleotides. In embodiments, the template nucleic acid is about 560 nucleotides. In embodiments, the template nucleic acid is about 570 nucleotides. In embodiments, the template nucleic acid is about 580 nucleotides. In embodiments, the template nucleic acid is about 590 nucleotides. In embodiments, the template nucleic acid is about 600 nucleotides. In embodiments, the template nucleic acid is about 610 nucleotides. In embodiments, the template nucleic acid is about 620 nucleotides. In embodiments, the template nucleic acid is about 630 nucleotides. In embodiments, the template nucleic acid is about 640 nucleotides. In embodiments, the template nucleic acid is about 650 nucleotides. In embodiments, the template nucleic acid is about 660 nucleotides. In embodiments, the template nucleic acid is about 670 nucleotides. In embodiments, the template nucleic acid is about 680 nucleotides. In embodiments, the template nucleic acid is about 690 nucleotides. In embodiments, the template nucleic acid is about 700 nucleotides. In embodiments, the template nucleic acid is about 1,600 nucleotides. In embodiments, the template nucleic acid is about 1,610 nucleotides. In embodiments, the template nucleic acid is about 1,620 nucleotides. In embodiments, the template nucleic acid is about 1,630 nucleotides. In embodiments, the template nucleic acid is about 1,640 nucleotides. In embodiments, the template nucleic acid is about 1,650 nucleotides. In embodiments, the template nucleic acid is about 1,660 nucleotides. In embodiments, the template nucleic acid is about 1,670 nucleotides. In embodiments, the template nucleic acid is about 1,680 nucleotides. In embodiments, the template nucleic acid is about 1,690 nucleotides. In embodiments, the template nucleic acid is about 1,700 nucleotides. In embodiments, the template nucleic acid is about 1,710 nucleotides. In embodiments, the template nucleic acid is about 1,720 nucleotides. In embodiments, the template nucleic acid is about 1,730 nucleotides. In embodiments, the template nucleic acid is about 1,740 nucleotides. In embodiments, the template nucleic acid is about 1,750 nucleotides. In embodiments, the template nucleic acid is about 1,760 nucleotides. In embodiments, the template nucleic acid is about 1,770 nucleotides. In embodiments, the template nucleic acid is about 1,780 nucleotides. In embodiments, the template nucleic acid is about 1,790 nucleotides. In embodiments, the template nucleic acid is about 1,800 nucleotides.

In embodiments, the template nucleic acid is a nucleic acid sequence. In embodiments the template nucleic acid is an RNA transcript. RNA transcripts are responsible for the process of converting DNA into an organism's phenotype, thus by determining the types and quantity of RNA present in a sample (e.g., a cell), it is possible to assign a phenotype to the cell. RNA transcripts include coding RNA and non-coding RNA molecules, such as messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target is pre-mRNA. In embodiments, the target is heterogeneous nuclear RNA (hnRNA). In embodiments the template nucleic acid is a single stranded RNA nucleic acid sequence. In embodiments, the template nucleic acid is an RNA nucleic acid sequence or a DNA nucleic acid sequence (e.g., cDNA). In embodiments, the template nucleic acid is a cDNA target nucleic acid sequence. In embodiments, the template nucleic acid is genomic DNA (gDNA), mitochondrial DNA, chloroplast DNA, episomal DNA, viral DNA, or complementary DNA (cDNA). In embodiments, the template nucleic acid is coding RNA such as messenger RNA (mRNA), and non-coding RNA (ncRNA) such as transfer RNA (tRNA), microRNA (miRNA), small nuclear RNA (snRNA), or ribosomal RNA (rRNA).

In embodiments, the template nucleic acids are RNA nucleic acid sequences or DNA nucleic acid sequences. In embodiments, the template nucleic acids are RNA nucleic acid sequences or DNA nucleic acid sequences from the same cell. In embodiments, the template nucleic acids are RNA nucleic acid sequences. In embodiments, the RNA nucleic acid sequence is stabilized using known techniques in the art. For example, RNA degradation by RNase should be minimized using commercially available solutions (e.g., RNA Later®, RNA Protect®, or DNA/RNA Shield®). In embodiments, the template nucleic acids are messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the template nucleic acid is pre-mRNA. In embodiments, the template nucleic acid is heterogeneous nuclear RNA (hnRNA). In embodiments, the template nucleic acid is mRNA, tRNA (transfer RNA), rRNA (ribosomal RNA), or noncoding RNA (such as lncRNA (long noncoding RNA)). In embodiments, the template nucleic acids are on different regions of the same RNA nucleic acid sequence. In embodiments, the template nucleic acids are cDNA target nucleic acid sequences and before step i), the RNA nucleic acid sequences are reverse transcribed to generate the cDNA target nucleic acid sequences. In embodiments, the template nucleic acids are not reverse transcribed to cDNA. When mRNA is reverse transcribed an oligo(dT) primer can be added to better hybridize to the poly A tail of the mRNA. The oligo(dT) primer may include between about 12 and about 25 dT residues. The oligo(dT) primer may be an oligo(dT) primer of between about 18 to about 25 nt in length.

In embodiments, the polynucleotide includes a gene or a gene fragment. In embodiments, the polynucleotide includes genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA). In embodiments, the polynucleotide includes messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA).

In an aspect is provided a solid support including a fraction of the plurality amplification products of any one of the aspects and embodiments herein. In embodiments, the fraction of the plurality of amplification products includes about 90%, about 91%, about 92%, about 93%, about 94%, about 95%, about 96%, about 97%, about 98%, about 99%, or about 99.5% of the plurality of amplification products. In embodiments, the fraction of the plurality of amplification products includes about 90% of the plurality of amplification products. In embodiments, the fraction of the plurality of amplification products includes about 95% of the plurality of amplification products. In embodiments, the fraction of the plurality of amplification products includes about 96% of the plurality of amplification products. In embodiments, the fraction of the plurality of amplification products includes about 97% of the plurality of amplification products. In embodiments, the fraction of the plurality of amplification products includes about 98% of the plurality of amplification products. In embodiments, the fraction of the plurality of amplification products includes about 99% of the plurality of amplification products. In embodiments, the fraction of the plurality of amplification products includes between about 99% and 99.5% of the plurality of amplification products. In embodiments, the fraction of the plurality of amplification products includes between about 99% and 99.9% of the plurality of amplification products. In embodiments, the fraction of the plurality of amplification products includes a template complex, as described herein.

In embodiments, each of the plurality of immobilized oligonucleotides (e.g., immobilized primers) is about 5 to about 25 nucleotides in length. In embodiments, each of the plurality of immobilized oligonucleotides (e.g., immobilized primers) is about 10 to about 40 nucleotides in length. In embodiments, each of the plurality of immobilized oligonucleotides (e.g., immobilized primers) is about 5 to about 100 nucleotides in length. In embodiments, each of the plurality of immobilized oligonucleotides (e.g., immobilized primers) is about 20 to 200 nucleotides in length. In embodiments, each of the plurality of immobilized oligonucleotides (e.g., immobilized primers) about or at least about 5, 6, 7, 8, 9, 10, 12, 15, 18, 20, 25, 30, 35, 40, 50 or more nucleotides in length. In embodiments, one or more immobilized oligonucleotides include blocking groups at their 3′ ends that prevent polymerase extension. A blocking moiety prevents formation of a covalent bond between the 3′ hydroxyl moiety of the nucleotide and the 5′ phosphate of another nucleotide. In embodiments, the 3′ modification is a 3′-phosphate modification, including a 3′ phosphate moiety, which is removed by a PNK enzyme or a phosphatase enzyme. Alternatively, abasic site cleavage with certain endonucleases (e.g., Endo IV) results in a 3′-OH at the cleavable site from the 3′-diesterase activity.

In embodiments, the immobilized oligonucleotides include one or more phosphorothioate nucleotides. In embodiments, the immobilized oligonucleotides include a plurality of phosphorothioate nucleotides. In embodiments, about or at least about 20%, 30%, 40%, 50%, 60%, 70%, 80%, 90%, or about 100% of the nucleotides in the immobilized oligonucleotides are phosphorothioate nucleotides. In embodiments, most of the nucleotides in the immobilized oligonucleotides are phosphorothioate nucleotides. In embodiments, all of the nucleotides in the immobilized oligonucleotides are phosphorothioate nucleotides. In embodiments, none of the nucleotides in the immobilized oligonucleotides are phosphorothioate nucleotides. In embodiments, the 5′ end of the immobilized oligonucleotide includes one or more phosphorothioate nucleotides. In embodiments, the 5′ end of the immobilized oligonucleotide includes between one and five phosphorothioate nucleotides.

In embodiments, the first and second polynucleotides are each attached to the solid support (i.e., immobilized on the surface of a solid support). The polynucleotide molecules can be fixed to surface by a variety of techniques, including covalent attachment and non-covalent attachment. In embodiments, the polynucleotides are confined to an area of a discrete region (referred to as a cluster). The discrete regions may have defined locations in a regular array, which may correspond to a rectilinear pattern, circular pattern, hexagonal pattern, or the like. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. Interstitial regions will typically have a surface material that differs from the surface material of the features on the surface. For example, features of an array can have polynucleotides that exceeds the amount or concentration present at the interstitial regions. In some embodiments the polynucleotides and/or primers may not be present at the interstitial regions. In embodiments, at least two different primers are attached to the solid support (e.g., a forward and a reverse primer), which facilitates generating multiple amplification products from the first extension product or a complement thereof.

In embodiments of the methods and compositions provided herein, the clusters have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 μm or a number or a range between any two of these values. In embodiments, the mean or median separation is about 0.1-10 microns. In embodiments, the mean or median separation is about 0.25-5 microns. In embodiments, the mean or median separation is about 0.5-2 microns. In embodiments, the mean or median separation is about or at least about 0.1 μm. In embodiments, the mean or median separation is about or at least about 0.25 μm. In embodiments, the mean or median separation is about or at least about 0.5 μm. In embodiments, the mean or median separation is about or at least about 1.0 μm. In embodiments, the mean or median separation is about or at least about 2.0 μm. In embodiments, the mean or median separation is about or at least about 5.0 μm. In embodiments, the mean or median separation is about or at least about 10 μm. The mean or median separation may be measured center-to-center (i.e., the center of one cluster to the center of a second cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm.

In embodiments of the methods provided herein, the amplicon clusters have a mean or median diameter of about 100-2000 nm, or about 200-1000 nm. In embodiments, the mean or median diameter is about 100-3000 nanometers, about 500-2500 nanometers, about 1000-2000 nanometers, or a number or a range between any two of these values. In embodiments, the mean or median diameter is about or at most about 100, 200, 300, 400, 500, 600, 700, 800, 900, 1,000, 1,100, 1,200, 1,300, 1,400, 1,500, 1,600, 1,700, 1,800, 1,900, 2000 nanometers or a number or a range between any two of these values. In embodiments, the mean or median diameter is about 100-3,000 nanometers. In embodiments, the mean or median diameter is about 100-2,000 nanometers. In embodiments, the mean or median diameter is about 500-2500 nanometers. In embodiments, the mean or median diameter is about 200-1000 nanometers. In embodiments, the mean or median diameter is about 1,000-2,000 nanometers. In embodiments, the mean or median diameter is about or at most about 100 nanometers. In embodiments, the mean or median diameter is about or at most about 200 nanometers. In embodiments, the mean or median diameter is about or at most about 500 nanometers. In embodiments, the mean or median diameter is about or at most about 1,000 nanometers. In embodiments, the mean or median diameter is about or at most about 2,000 nanometers. In embodiments, the mean or median diameter is about or at most about 2,500 nanometers. In embodiments, the mean or median diameter is about or at most about 3,000 nanometers.

In embodiments, the kit includes components useful for cleaving and/or digesting a fraction of a plurality of template polynucleotides. In embodiments, the kit includes a cleaving agent (e.g., a cleaving agent that specifically recognizes a cleavable site). In embodiments, the kit includes buffers and associated reagents for performing a cleavage reaction with the cleaving agent. In embodiments, the cleaving agent includes a reducing agent, sodium periodate, Rnase, Formamidopyrimidine DNA Glycosylase (Fpg), endonuclease, or uracil DNA glycosylase (UDG). Examples of cleaving agents include DNA repair enzymes, glycosylases, DNA cleaving endonucleases, or ribonucleases. For example, cleavage at dUTP may be achieved using uracil DNA glycosylase and endonuclease VIII (USER™, NEB, Ipswich, Mass.), as described in U.S. Pat. No. 7,435,572. In embodiments, when the modified nucleotide is a ribonucleotide, the cleavable site can be cleaved with an endoribonuclease. In embodiments, cleaving an extension product includes contacting the cleavable site with a cleaving agent, wherein the cleaving agent includes a reducing agent, sodium periodate, RNase, formamidopyrimidine DNA glycosylase (Fpg), endonuclease, restriction enzyme, or uracil DNA glycosylase (UDG). In embodiments, the cleaving agent is an endonuclease enzyme such as nuclease P1, AP endonuclease, T7 endonuclease, T4 endonuclease IV, Bal 31 endonuclease, Endonuclease I (endo I), Micrococcal nuclease, Endonuclease II (endo VI, exo III), nuclease BAL-31 or mung bean nuclease. In embodiments, the cleaving agent includes a restriction endonuclease, including, for example a type IIS restriction endonuclease. In embodiments, the cleaving agent is an exonuclease (e.g., RecBCD), restriction nuclease, endoribonuclease, exoribonuclease, or RNase (e.g., RNAse I, II, or III). In embodiments, the cleaving agent is a restriction enzyme. In embodiments, the cleaving agent includes a glycosylase and one or more suitable endonucleases.

In embodiments, the kit includes a single restriction endonuclease. In embodiments, the restriction endonuclease may include XbaI, EcoRI-HF, NheI, BamHI, XcmI, PflMI, BstEII, NcoI, HpaI, BsgI, AfeI, StuI, BsrGI, or a CRISPR-Cas9 nuclease (e.g., to achieve an approximate 95% cleavage or digestion rate, or the cleaving activity, as described by Zhang et al (see, Zhang Y et al. PLoS ONE. 2020. 15(12): e0244464, which is incorporated herein by reference in its entirety)). In embodiments, the restriction endonuclease may include XbaI, EcoRI, BamHI, XcmI or BstEII (e.g., to achieve an approximate 98% or greater cleavage or digestion rate, or the cleaving activity, as described by Zhang et al.). In embodiments, the restriction endonuclease may include EcoRI or XbaI (e.g., to achieve an approximate 99% or greater cleavage or digestion rate, or the cleaving activity, as described by Zhang et al.).

In embodiments, the kit includes a programmable endonuclease. In embodiments, the kit further includes a guide oligonucleotide (e.g., a guide oligonucleotide that complexes with the programmable endonuclease and targets the programmable endonuclease to a target nucleic acid sequence). In embodiments, the programmable endonuclease is an argonaute enzyme. In embodiments, the argonaute enzyme is Thermus thermophilus argonaute (TtAgo), or a mutant thereof. In embodiments, the programmable endonuclease is from the haloalkaliphilic archaebacterium N. gregoryi SP2 (NgAgo), or a mutant thereof.

### EXAMPLES

### Example 1. Monoclonal Clustering

Next generation sequencing (NGS) methods often rely on the amplification of genomic fragments hybridized to polynucleotide primers on a solid surface, referred to as amplification sites. Ideally these amplification sites have one initial template fragment at a given feature (e.g., site on a flow cell, such as within a well, on a particle, or both on a particle in a well) that is then amplified to occupy the entire feature. However, instances of polyclonal sites, (i.e., where more than one distinct polynucleotide is present and amplified) negatively impact sequencing results by increasing sequencing duplications or simultaneous interfering signaling.

Hybridizing a target polynucleotide to a polynucleotide primer is an inherently stochastic event. For stochastic events occurring over a period of time (e.g., a seeding-amplification cycle) it may be convenient to use the Poisson approximation to better understand the probability of an event occurring during that time. For example, if one knows the average rate of a hybridizing event, represented as λ^{seed}, (i.e., how often a target polynucleotide hybridizes to a polynucleotide primer) occurring during a seeding-amplification cycle, it is possible to calculate the probability that an amplification site will contain an amplicon (e.g., a monoclonal amplicon) following a seeding-amplification cycle. Two variables affecting λ^{seed} include the concentration target polynucleotide and the amount of time the target polynucleotide is exposed to the polynucleotide primer, t_{seed}, during a seeding-amplification cycle. Generally, increasing the concentration of the target polynucleotide or increasing t_{seed} increases λ_{seed}.

Conventional methods typically overseed an array of available sites, that is, the methods typically used ensure the concentration of the target polynucleotides are in abundance relative to the available amplification sites to maximize the opportunity for a target polynucleotide to hybridize to the primer in the amplification site. Unfortunately, this results in polyclonal amplicons (i.e., two or more populations of distinct fragment amplicons) forming in the amplification site. Polyclonal amplicons result in poor quality sequencing due to the fact that multiple templates are present, in contrast to monoclonal clusters, which have only one template per spot (i.e., one template per feature). Increasing the proportion of monoclonal clusters on a solid support, such as a flow cell, for example, will increase the total read output of a sequencing run, increase the confidence of a correctly called base therefore increasing the quality score (i.e., accuracy), and reduce the cost per sequencing read.

Existing methods to overcome polyclonality have been described, and include kinetic exclusion amplification (see, e.g., U.S. Pat. Pubs. US2017/0335380 and US2018/0037950, each of which are incorporated herein by reference), which involves the use of an amplification reaction wherein the seeding process proceeds at a slower rate than the clustering process. Seeded spots are fully clustered before they might be reseeded by a different template. Kinetic exclusion amplification requires that the number of target nucleic acids in the seeding solution be greater than the number of spots that may be seeded. An alternative method, referred to herein as staircase amplification (see, e.g., U.S. Pat. Pub. US2018/0044732, which is incorporated herein by reference, relies on repeated rounds of template seeding and clustering of a subset of flow cell spots to increase the seeding density and reduce polyclonality.

Embodiments of the invention described herein make significant advances over existing clustering methods (e.g., staircase amplification and kinetic exclusion amplification) and produce a higher fraction of monoclonal clusters. The methods of the invention herein are referred to as “bottleneck clustering”, and include seeding templates onto a subset of the primers on a flow cell, amplifying the subset over a defined number of amplification cycles, and then randomly removing (e.g., randomly digesting and/or cleaving) the templates (referred to herein as S-AD clustering). The amplification and removal steps are then repeated over a defined number of cycles to decrease polyclonal clusters, promoting a high percentage of monoclonal amplicons. Alternative embodiments of the invention include seeding templates onto a subset of the primers on a flow cell, followed by randomly removing (e.g., randomly digesting and/or cleaving) the seeded templates, and then amplifying the remaining seeded templates (referred to herein as SDA clustering). Repeating this process also leads to significant proportions of monoclonal amplicons on a solid support (e.g., a flow cell).

### Example 2. Bottleneck Clustering

As described supra, amplification sites on a solid support ideally have one copy (i.e., are monoclonal) of a hybridized polynucleotide fragment, however instances of polyclonal sites, (i.e., where more than one distinct polynucleotide is present) are common and interfere with sequencing results. Increasing the proportion of monoclonal clusters on a flow cell, for example, will increase the total quality and read output of a sequencing run, and reduce the cost per read.

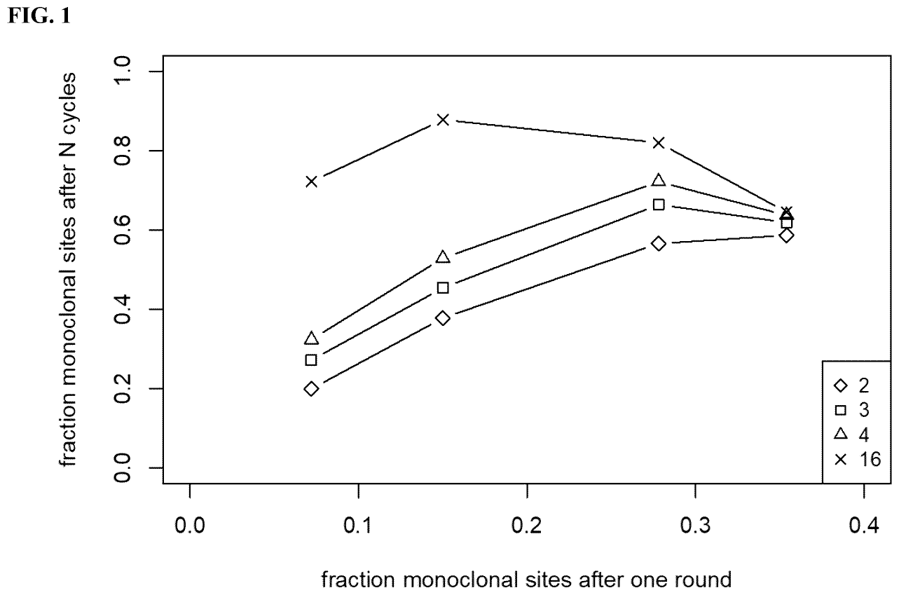

Existing methods to overcome polyclonality include kinetic exclusion amplification which involves the use of an amplification reaction wherein the seeding process proceeds at a slower rate than the clustering process. Seeded spots are fully clustered before they might be reseeded, reducing polyclonality. Kinetic exclusion amplification requires that the number of target nucleic acids in the seeding solution be greater than the number of spots that may be seeded. An alternative method, referred to herein as staircase amplification, relies on repeated rounds of template seeding and clustering of a subset of flow cell spots to increase the seeding density and reduce polyclonality. FIG. 1 is a graph illustrating the results of a computer simulation of staircase amplification to describe how the monoclonal occupancy of wells on a solid support (e.g., a patterned flow cell) can vary depending on initial seeding conditions (e.g., by fraction of monoclonal sites after one round of seeding) and the number of seeding events (2 to 16 events modeled). As shown, the performance of staircase amplification was found to be highly dependent on the seeding probability and thus the library concentration.

### Seeding, Amplification, and Digestion (S-AD) Bottleneck Clustering

FIGS. 2A-2C illustrate one embodiment of bottleneck clustering for generating monoclonal clusters, including the steps of seeding templates onto a solid support, amplification to generate clusters, and random removal of the majority of the templates, followed by repeating the amplification and removal steps (also referred to herein as the S-AD method). FIG. 2A illustrates the steps of randomly seeding a diverse population of templates onto a plurality of immobilized primers. Following seeding, cluster amplification is performed to generate a polyclonal cluster containing, for example, 3 different template species. FIG. 2B illustrates the steps of randomly removing the majority of the templates from the polyclonal cluster (e.g., via digestion), followed by more cycles of amplification to regenerate the cluster. In this example, the polyclonal cluster now contains two different species of templates. FIG. 2C illustrates the steps of randomly remove the majority of the templates from the polyclonal cluster (e.g., via digestion), resulting in a single species of template. Another round of amplification results in monoclonal cluster. Depending on the diversity of the template pool, repeating the removing and amplifying steps is optional, and may be performed one or more times to result in highly monoclonal clusters.

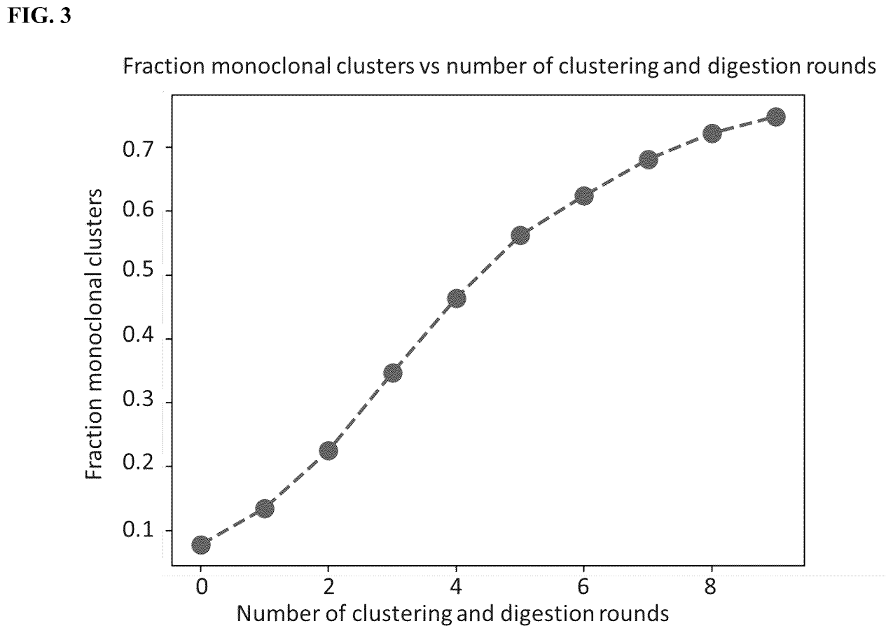

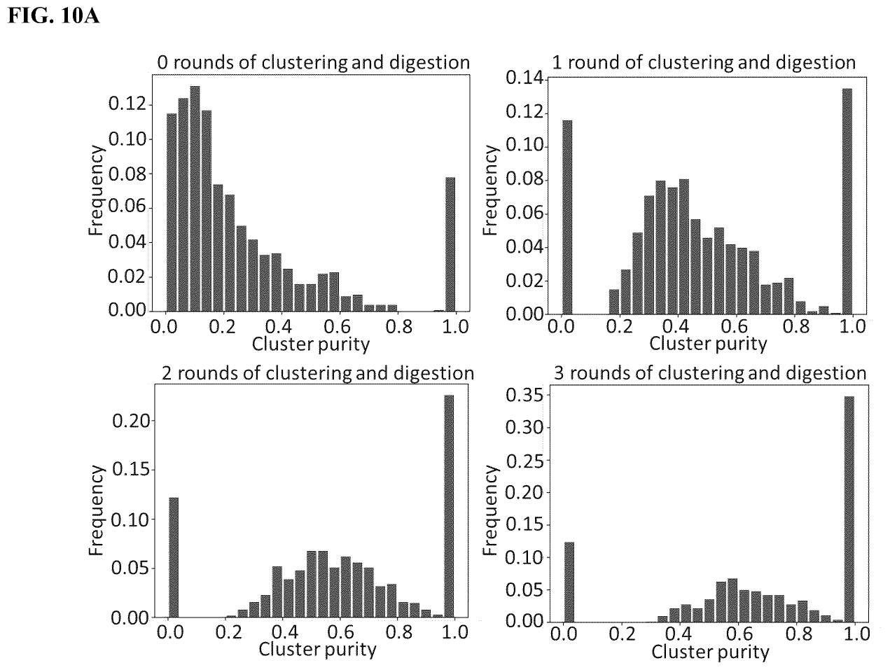

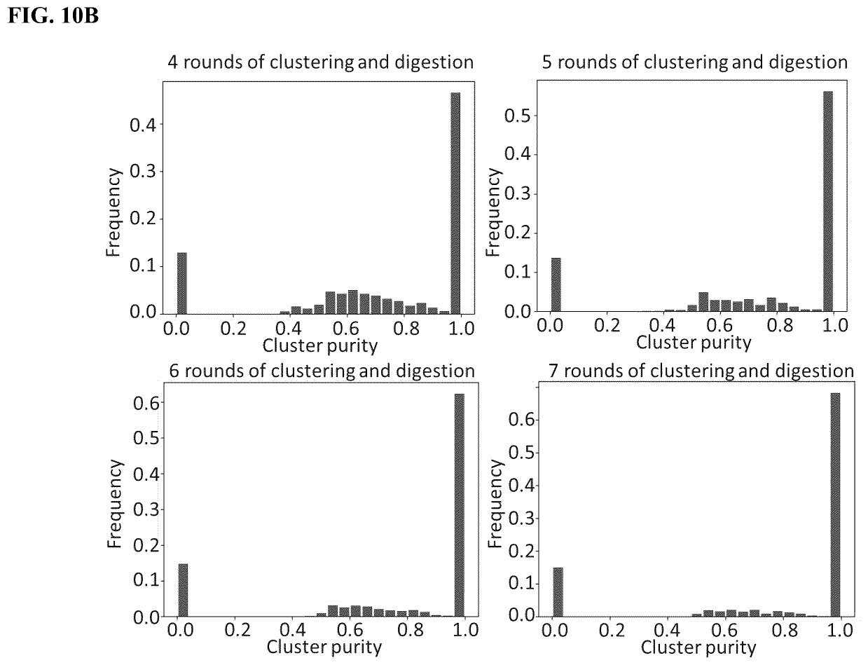

Shown in FIG. 3 are the results of a computer simulation to determine the fraction of monoclonal clusters versus the number of cluster amplification and digestion rounds (e.g., 8 rounds) performed for the S-AD method, as described in FIGS. 2A-2C. 1,000 spots were simulated, with each spot containing up to 1000 templates following cluster amplification. During seeding, simulation parameters included a 90% probability that a spot is seeded with at least one template. During the digestion step, simulation parameters included that 99.5% of the templates were digested during each removal step. The simulation shows a clear trend between increasing the number of rounds of clustering and digestion performed and the resulting increasing fraction of monoclonal clusters. In line with the results of FIG. 3, FIGS. 10A-10B show a series of histograms comparing the purity per cluster (i.e., the fraction of cluster templates including the most abundant cluster species) over several rounds of bottleneck clustering. FIG. 10A illustrates the purity per cluster following 0, 1, 2, and 3 rounds of bottleneck clustering. FIG. 10B illustrates the purity per cluster following 4, 5, 6, and 7 rounds of bottleneck clustering. These results clearly show that successive rounds of bottleneck clustering lead to significant levels of monoclonal/pure clusters.

### Seeding, Digestion, and Amplification (SDA) Bottleneck Clustering

An alternative embodiment of bottleneck clustering is described herein, and illustrated in FIGS. 4A-4C. This embodiment includes repeating the steps of seeding a population of templates onto a solid support, randomly removing a majority of the templates, and amplification (also referred to herein as the SDA method). FIG. 4A illustrates the steps of randomly seeding a diverse population of templates onto a plurality of immobilized primers. Following seeding, random removal of the majority of the templates from the polyclonal cluster (e.g., via digestion) is performed, followed by cycles of amplification to amplify the polyclonal cluster. FIG. 4B illustrates the steps of re-seeding a population of templates onto the plurality of immobilized primers, followed by randomly removing the majority of the templates from the polyclonal cluster (e.g., via digestion). In this example, the polyclonal cluster now contains one species of templates. FIG. 4C illustrates the step of performing another round of amplification, resulting in a monoclonal cluster.

### Approaches for Randomly Removing a Majority of the Templates.

In embodiments, partial template digestion is achieved by calibrating the digestion time and amount of restriction enzyme used for digestion. The calibration occurs, for example, by performing an experiment where a fully clustered flow cell is subjected to digestion over a range of times and with varying amount of restriction enzyme. Following digestion, the amount of DNA remaining bound to the flow cell is quantified. The resultant 2×2 matrix of digestion efficiencies is used to select the ideal combination of enzyme amount and digestion time to achieve the desired level of digestion. This approach may be preferred when employing the SDA approach, where the negative effects of potential over-digestion are mitigated by reseeding.

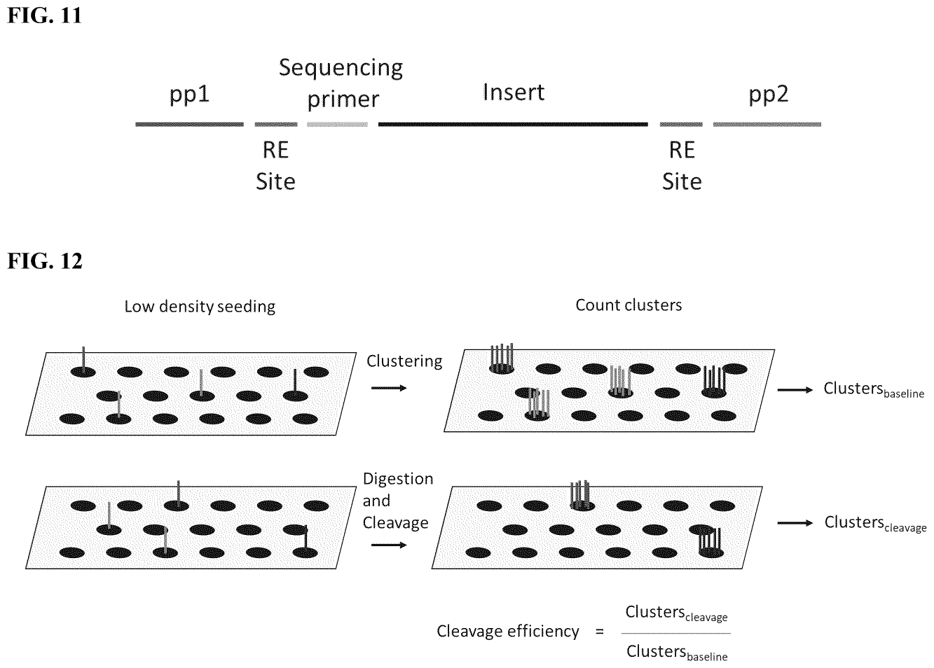

For example, first a human whole-genome library is prepared containing BglII restriction sites flanking the pp1 and pp2 adapter sequences (as illustrated in FIG. 11). The library is then seeded on a flow cell. A series of BglII restriction enzyme digestion time courses can be run initially to determine the digestion time required to remove approximately 80% of the seeded templates, which can be determined using a FAM-labeled probe complementary to the pp1/pp2 sequence. Once the optimal BglII digestion time has been determined, a new flow cell is prepared and seeded with the library. The seeded library is digested using BglII such that approximately 80% of the templates are removed. Then, the remaining templates are amplified over 5 rounds of chemical bridge PCR (cbPCR). The template seeding, BglII digestion, and cbPCR steps are then repeated 5 times, with a FAM-probe detection step after each round of BglII digestion to estimate the amount of remaining template. The detected FAM signal should increase following each successive round of seeding, digestion, and amplification, indicating a growing cluster. Additional amplification of the clusters may subsequently be performed to achieve desired cluster brightness (as determined by the FAM probe). Using the bottleneck clustering conditions determined supra, a fresh flow cell is then prepared, seeding the BglII site-containing template library on the flow cell at a density such that approximately all sports are seeded. Digestion is then performed with BglII and a digestion time required to remove approximately 80% of the templates. This is followed by 5 rounds of cbPCR amplification. The seeding, digestion, and amplification steps are then repeated another 5 rounds, with additional amplification performed as needed. The predominantly monoclonal clusters are then sequenced according to methods known in the art (e.g., SBS).

In embodiments, in place of a restriction enzyme one could use a Thermus thermophilus argonaute (TtAgo) protein in combination with a guide DNA. The TtAgo protein is a DNA-endonuclease which requires a short 5′-phosphorylated single-stranded DNA guide to target its activity to a specific corresponding sequence on a substrate. TtAgo introduces one break in the phosphodiester backbone of the complementary substrate sequence.

Alternatively, a methylation sensitive restriction enzyme in combination with a spike in of methylated C's at a desired ratio during clustering provides a given frequency of methylated restriction sites, which are resistant to cleavage. For example, the library molecules include adapters with a methylation sensitive NotI restriction site (8 bp recognition motif, methylcytosine sensitive), as shown in FIG. 11. Clustering is performed using dNTPs where dCTP and 5-methyl dCTP are used at a 10:1 ratio. Approximately 10% of cytosine positions will be methylated, which corresponds to about 10% of NotI sites are methylated and thus resistant to enzymatic cleavage. Approximately 1% of the templates will not have a NotI site cleaved during digestion. Following the final digestion step, one may regenerate clusters using 100% dCTP. This will create fully unmethylated templates for sequencing and avoid potential issues in reading of methylcytosine during sequencing. In embodiments, a single NotI site is included in only one of the two adapters used when generating a library and clustering is performed using dNTPs where dCTP and 5-methyl dCTP are used at a 100:1 ratio.

In embodiments, during amplification (i.e., clustering) photocaged dNTPs are used in place of a standard dNTP in combination with restriction enzyme digestion. Examples of photocages dNTPs are found in Boháčová et al. Org. Biomol. Chem., 2018, 16, 1527-1535 and Vaníková and Hocek, Angewandte Chemie Volume 53, Issue 26; (2014) 6734-6737. Incorporation of the photocaged dNTP into the restriction enzyme cut site renders the site resistant to cleavage. The caging may be removed by brief application of UV light. In this system the rate of digestion may be regulated by the duration of the application of UV light prior to digestion.

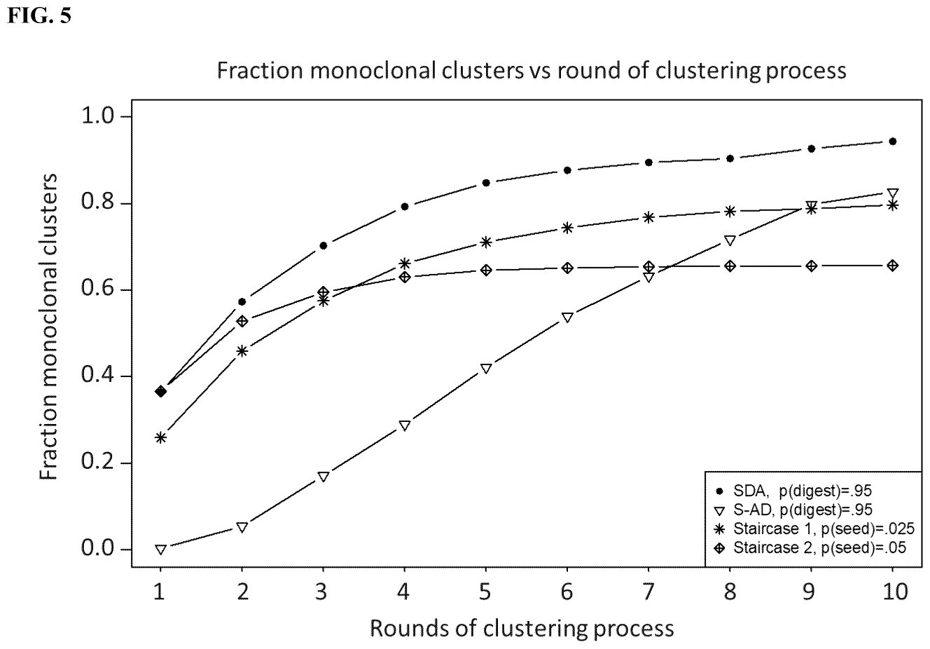

Illustrated in FIG. 5 is a computer simulation comparing the performance of the SDA, S-AD, and staircase amplification methods using the best performing conditions for each method as determined by the fraction of monoclonal spots after each round of the process. Staircase 1 corresponds to the seeding condition that produced the highest fraction of monoclonal clusters after 10 rounds of the process, while Staircase 2 corresponds to the seeding condition that produced the highest fraction of monoclonal clusters after 2 rounds of the process. p(seed) represents the probability of seeding a subset of the spots (e.g., 5% probability of seeding a spot), and p(digest) represents the probability of randomly removing a template in a spot (e.g., 95% probability of removing a template). The simulation suggests that the SDA embodiment of bottleneck clustering delivers a larger fraction of monoclonal spots than staircase amplification at each round of the process.

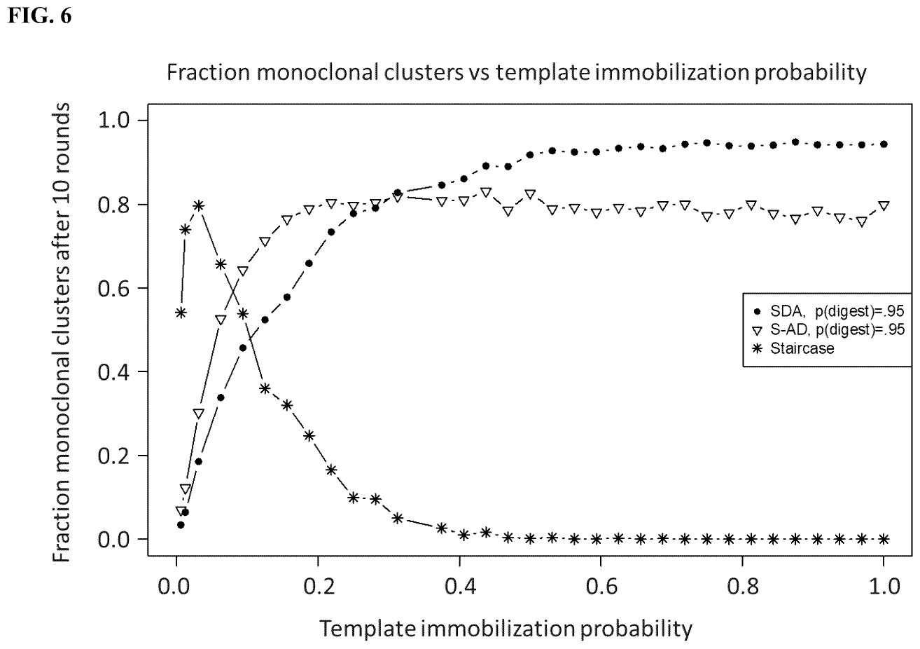

As shown in FIG. 6, bottleneck clustering was found to deliver optimal results with respect to monoclonal clustering over a wide range of template seeding inputs, unlike staircase amplification, which as mentioned supra, is dependent on seeding. This computer simulation compared the fraction of monoclonal features of the SDA, S-AD, and staircase amplification methods after 10 rounds of the process as a function of the relative seeding input (e.g., seeding probability). The performance of staircase amplification is highly dependent on seeding probability, unlike the embodiments of bottleneck clustering, SDA and S-AD. Consequently, bottleneck clustering is expected to be more robust to library quantification inaccuracies compared to staircase amplification. Both the SDA and S-AD embodiments were superior to staircase amplification in terms of fraction of monoclonal clusters, with the SDA embodiment of bottleneck clustering performing best.

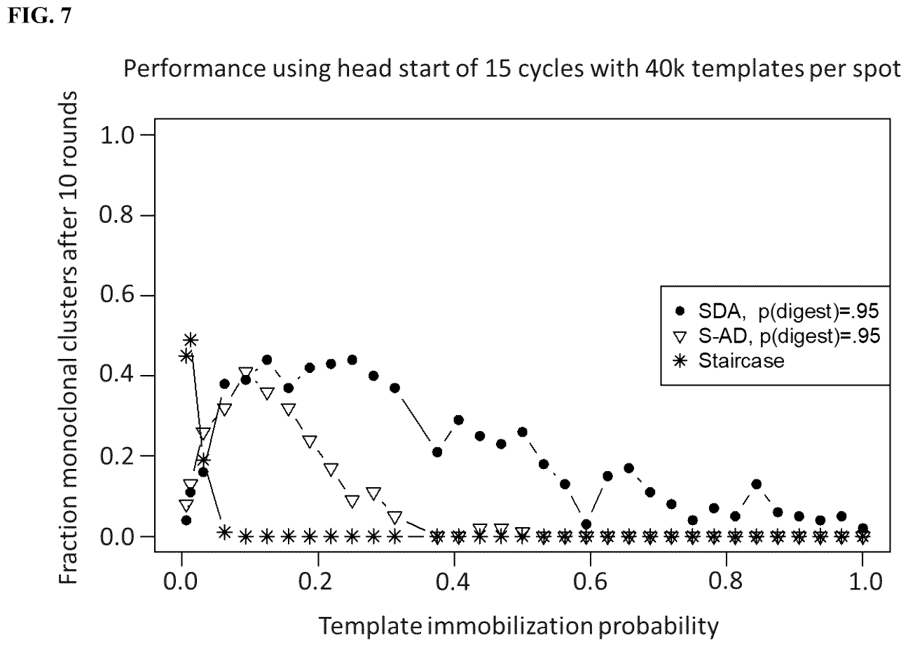

In addition, it was found that the spot size is a tunable parameter for all amplification methods tested, as shown in FIG. 7. This computer simulation compared the fraction of monoclonal features of the SDA, S-AD, and staircase amplification methods after 10 rounds of the process as a function of the relative seeding input (e.g., seeding probability), with the number of templates per spot increased from 100 to 40,000 templates. Additionally, 15 PCR amplification cycles per round we performed in between seeding events. In this example, the fraction of monoclonal features indicates the fraction of spots in the simulation having >95% template purity. With this number of PCR cycles, it was found that spots were not fully clustered after each round of the process, resulting in a drop in performance. This result highlights how providing 5-15 cycles between seeding events is insufficient to achieve super Poisson clustering under these examined conditions. Rather, one must completely or almost completely cluster spots between rounds (i.e., perform 20, 30, 40, or more PCR amplification cycles between seeding).

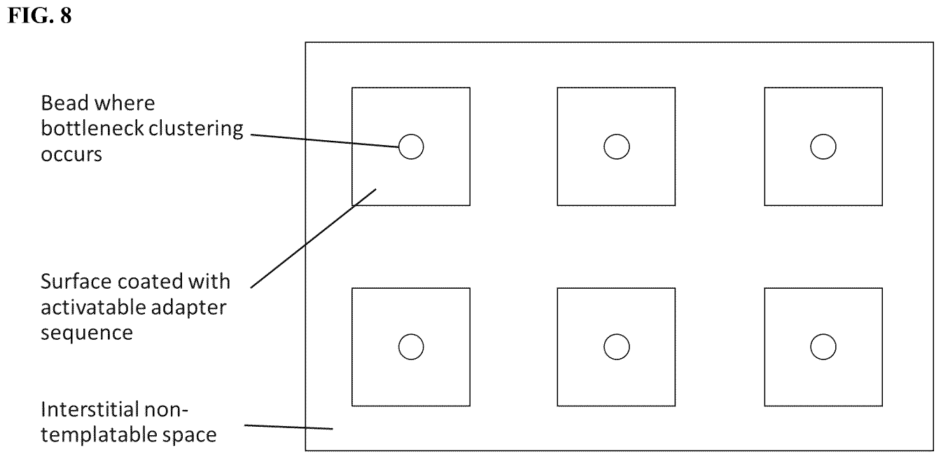

FIG. 8 illustrates an embodiment of the invention to reduce the number of PCR cycles required per round of clustering while maintaining large clusters (e.g., ˜40k template molecules per spot). A solid support (e.g., a patterned flow cell) is produced, and includes particles (e.g., the particles shown in the center of each square feature) containing a limited number of adapter oligos (e.g., 50, 100, or 1,000 adapter molecules, or a template copy number that is insufficient for sequencing) deposited within larger sub-surfaces (squares above) containing activatable adapter oligos. The sub-surfaces are separated by non-templatable interstitial space. Clustering according to the methods described herein is performed on the surface of the particles, enabling the production of monoclonal particles with a minimal number of PCR cycles. Following clustering, the activatable surface is rendered active (e.g., by cleavage/digestion of a blocking moiety) and the monoclonal templates on the particles are copied onto the activatable surface, thereby producing large monoclonal colonies. Cluster separation improves signal deconvolution and minimizes spreading linked to ‘optical’ duplicates.

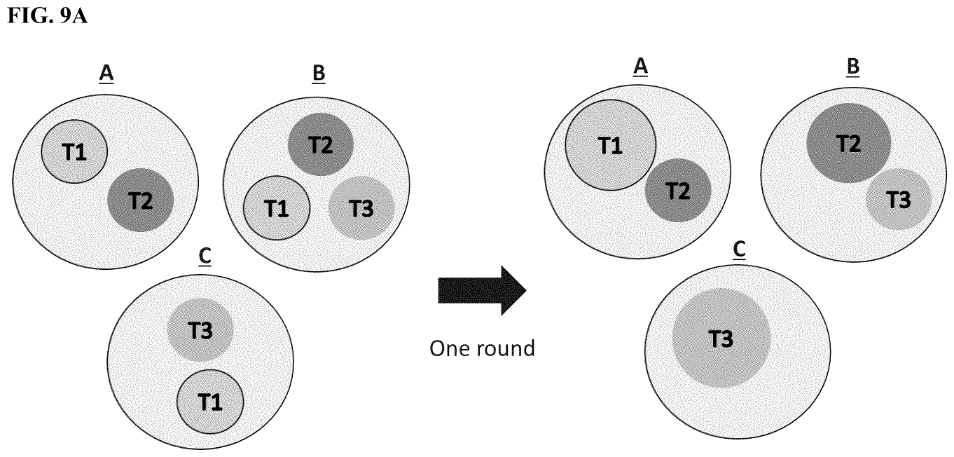

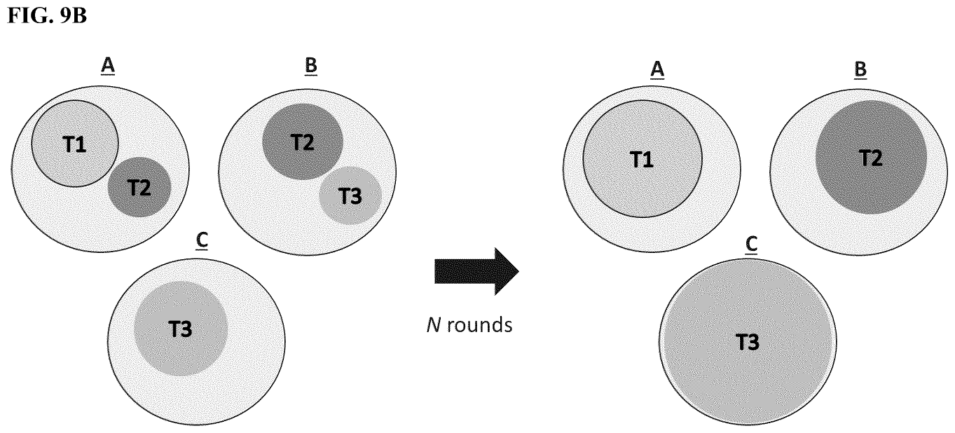

FIGS. 9A-9B illustrates competition between different templates for clonal dominance on individual spots following bottleneck clustering. FIG. 9A shows three spots, A, B, and C, wherein spot A contains two immobilized templates (T1 and T2), spot B contains three immobilized templates (T1, T2, and T3), and spot C includes two immobilized templates (T1 and T3). Following, for example, one round of a bottleneck clustering process as described herein, the quantity of T1 template has grown in spot A, while T1 template in spot B is removed, e.g., during digestion, allowing T2 template to increase in quantity. T3 template in spot C has also increased in quantity, with T1 template also being lost in spot C (i.e., forming a monoclonal T3 cluster in spot C). FIG. 9B illustrates the generation of monoclonal clusters following N rounds of bottleneck clustering. For example, spot A has only T1 template, spot B has only T2 template, and spot C continues to have only T3 template. With subsequent rounds of clustering, T3 template in spot C continues to grow in density.

### PROGRAMMABLE ENDONUCLEASE BOTTLENECK CLUSTERING

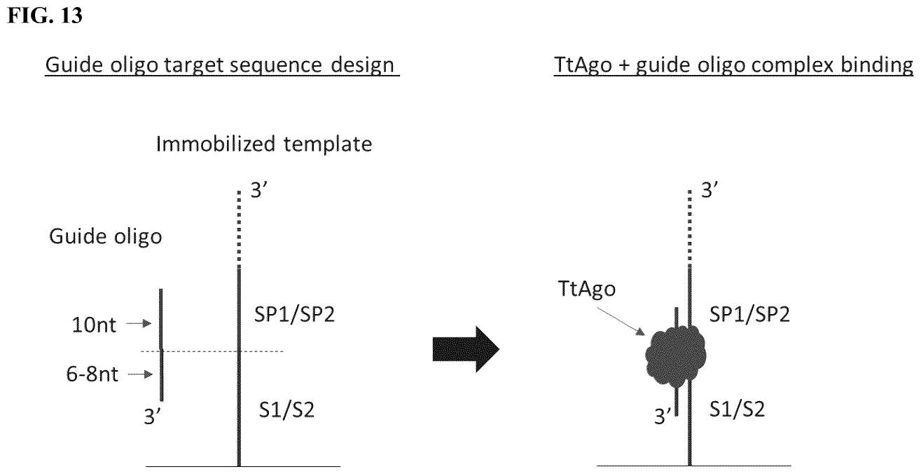

An alternative embodiment of bottleneck clustering is described herein, and illustrated in FIGS. 13-15. This embodiment takes advantage of the selective targeting of a programmable endonuclease enzyme, such as an argonaute enzyme, complexed to a guide oligonucleotide, wherein the argonaute-guide oligo complex is targeted towards a subset of immobilized template polynucleotides for cleavage and removal. For example, the guide oligonucleotide is complementary to a portion of a platform primer sequence and a portion of a sequencing primer binding sequence of an immobilized template polynucleotide, such that the argonaute enzyme is targeted towards the immobilized template polynucleotide. Following argonaute cleavage and removal of the majority of the immobilized template polynucleotides, additional rounds of amplification result in monoclonal cluster populations on a solid support.

FIG. 13 illustrates an embodiment of a programmable endonuclease-based approach for generating monoclonal clusters. A programmable endonuclease, for example, a Thermus thermophilus argonaut (TtAgo) enzyme and an associated guide oligo, with a length of between 16 to 18 nucleotides, are used to target immobilized templates. TtAgo cleaves a complementary polynucleotide between the bases corresponding to positions 10 and 11 of the DNA guide oligo. With respect to an immobilized template sequence including a platform primer sequence (e.g., S1 or S2, or a complement thereof) and a sequencing primer binding sequence (e.g., SP1 or SP2, or a complement thereof), a TtAgo guide oligo is designed, for example, such that the first 10 nucleotide on the 5′ end of the guide oligo are complementary to the sequencing primer binding sequence, and the adjacent, downstream 6 to 8 nucleotides are complementary to the platform primer sequence, or complement thereof. Addition of a TtAgo and guide oligo complex result in targeting of the TtAgo complex to the immobilized template.

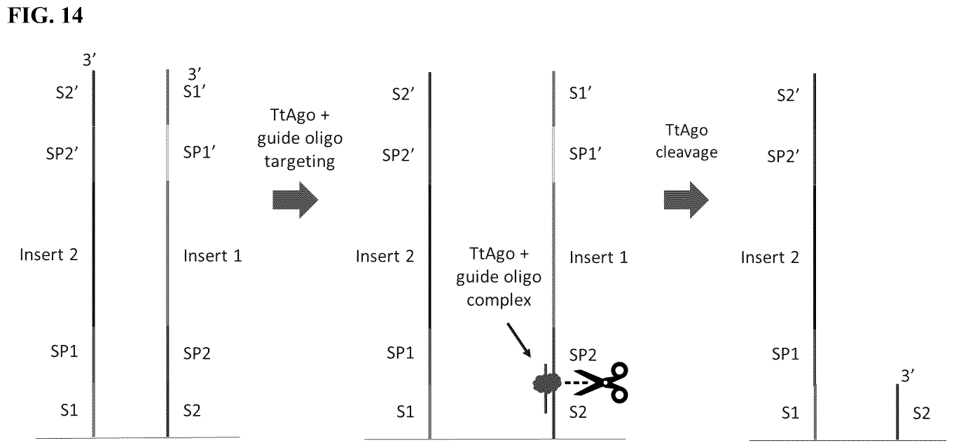

FIG. 14 illustrates additional steps of an embodiment of a programmable endonuclease-based approach for generating monoclonal clusters. A TtAgo and guide oligo complex (i.e., a TtAgo complex) specific for an immobilized template, as described in FIG. 13, is added to a solid support including a plurality of immobilized templates. In embodiments, more than one plurality of TtAgo complexes is added to the support, wherein the guide oligo of each plurality is complementary to a specific platform primer sequence and sequencing primer binding sequence combination. For example, 5 different pluralities of TtAgo complexes are added to a solid support including, for example, immobilized template polynucleotides including one of 6 different platform primer sequences, such that 1 out of the 6 platform primer sequences are targeted for cleavage by each TtAgo complex.

As illustrated in FIG. 14, a TtAgo complex is contacted to a solid support including a first immobilized template polynucleotide include, from 5′ to 3′, a first platform primer sequence (e.g., S1), a first sequencing primer binding sequence (e.g., SP1), an insert (e.g., insert 2), a first sequencing primer binding sequence complement (e.g., SP2′) and a first platform primer sequence complement (e.g., S2′), and a second immobilized template polynucleotide including, from 5′ to 3′, a second platform primer sequence (e.g., S2), a second sequencing primer binding sequence (e.g., SP2), an insert (e.g., insert 1), a second sequencing primer binding sequence complement (e.g., SP1′), and a second platform primer sequence complement (e.g., S1′). The complexed guide oligo is complementary to a portion of the second platform primer sequence and the second sequencing primer binding sequence, following a sequence complementarity scheme as described in FIG. 13. Following an incubation with the TtAgo complex, the second immobilized template polynucleotide is cleaved between the 10′ and 11′ nucleotides from the 5′ end of the second platform primer sequence, releasing the second template polynucleotide such that the solid support now includes a free second platform primer sequence (e.g., a second platform primer sequence with an extendable 3′ end) that may participate in additional amplification cycles with other immobilized template polynucleotide including a complementary platform primer binding sequence.

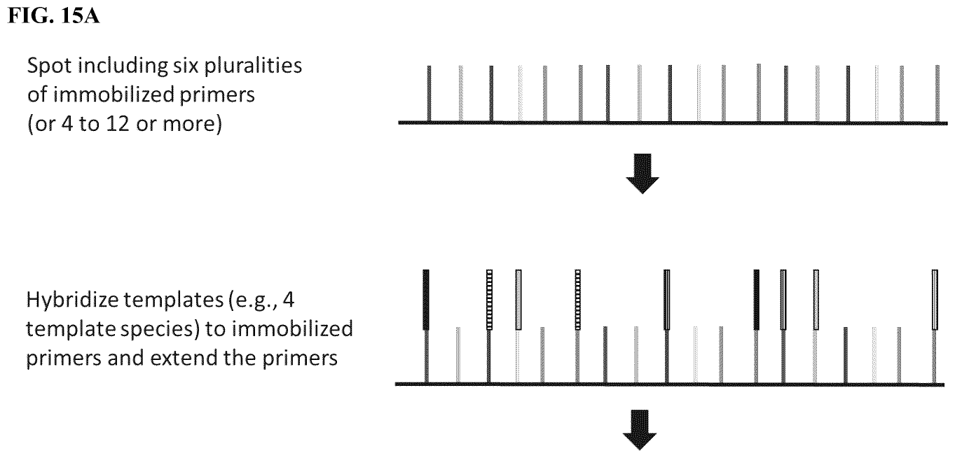

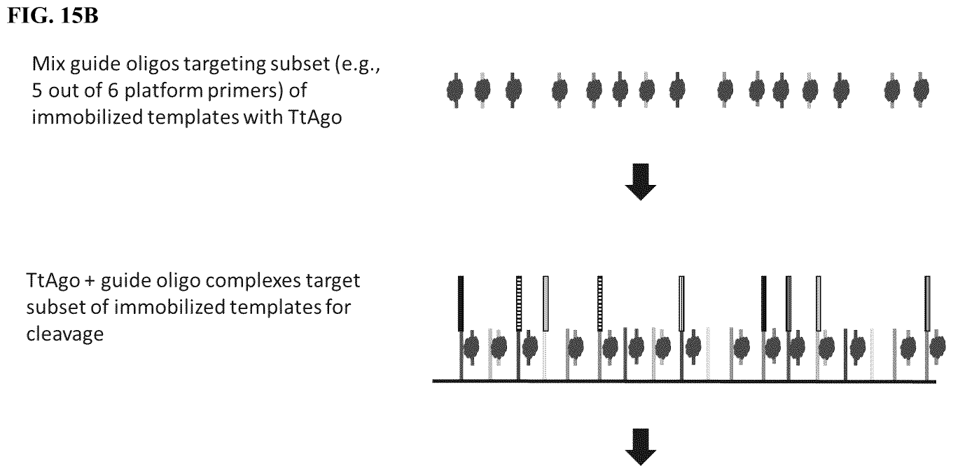

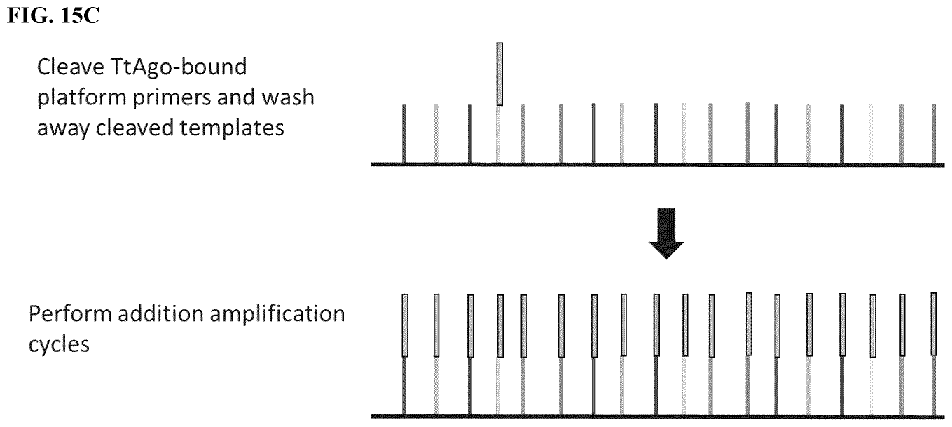

FIGS. 15A-15C illustrates an embodiment for generating monoclonal clusters using a programmable-based approach, including the steps of hybridizing templates onto a plurality of immobilized primers on a solid support, extending the primers, amplification to generate clusters, and selective removal of the majority of the templates, followed by repeating the amplification step (and optionally, the removal step). FIG. 15A illustrates the steps of randomly seeding (i.e., hybridizing templates to immobilized primers and extending the primers, thereby immobilizing the complements of the templates) a diverse population of templates onto a plurality of immobilized platform primers (e.g., 6 pluralities of immobilized platform primers, or between 4 to 12 or more pluralities of immobilized platform primers). Following seeding, cluster amplification is performed to generate a polyclonal cluster containing, for example, 4 different immobilized template species. FIG. 15B illustrates the steps of selective targeting the majority of the templates from the polyclonal cluster with a TtAgo complex (e.g., A TtAgo enzyme and guide oligo complex, wherein the guide oligo is specific for the platform primer sequence and sequencing primer binding sequence of one or more immobilized template polynucleotides, as described in FIGS. 13-14). Following targeting of a subset of the platform primer sequences (e.g., 5 out of 6 platform primer sequences), the TtAgo-bound templates are cleaved and removed, as shown in FIG. 15C. Additional rounds of amplification result in a monoclonal cluster.

These results highlight the benefits of the bottleneck clustering approaches and significant increase in monoclonal cluster formation that can be achieved. Reducing the distribution and frequency of polyclonal amplicons while increasing the density and proportion of monoclonal spots will result in significant improvements in sequencing throughput, accuracy, and reduced cost. In addition to increasing the throughput of sequencing chips, the method may be used as part of a chip production step to convert a conventional flow cell into a flow cell containing spots having one of a predetermined number of target specific oligonucleotide sequences. This would enable applications such as SNP sequencing for genotyping, large gene expression panels, and facilitate the production of customized targeted sequencing panels. The method described herein could also be used as part of the creation of DNA hybridization-based microarrays.

What is claimed is:

1. A method of amplifying a template polynucleotide on a solid support, said method comprising:
(i) executing one or more amplification cycles thereby forming a plurality of immobilized amplification products comprising a cleavable site on the solid support, wherein each amplification cycle comprises:
a) hybridizing the template polynucleotide to a first oligonucleotide, wherein said first oligonucleotide comprises a cleavable site and is attached to the solid support, and extending the first oligonucleotide with a polymerase to generate an immobilized complement of the template polynucleotide;
b) denaturing the template polynucleotide and immobilized complement;
c) hybridizing the immobilized complement to a second oligonucleotide, wherein said second oligonucleotide comprises a cleavable site and is attached to the solid support, and extending the second oligonucleotide with a polymerase to generate an immobilized copy of the template polynucleotide;
(ii) contacting a fraction of the cleavable sites with a cleaving agent to remove a fraction of the plurality of immobilized amplification products; and
(iii) after step (ii), executing one or more amplification cycles.

2. The method of claim 1, further comprising repeating steps (ii) and (iii).

3. The method of claim 1, prior to step (i), executing one or more sparse-seed cycles, wherein each sparse-seed cycle comprises contacting the solid support with a plurality of template polynucleotides and forming a plurality of template complexes, wherein each template complex comprises a template polynucleotide hybridized to an immobilized oligonucleotide comprising a cleavable site; contacting the template complexes with a polymerase and extending the immobilized oligonucleotide to form a plurality of extended complements of templates; and removing a fraction of the extended complements of templates.

4. The method of claim 1, prior to step (ii), executing 2 to 20 amplification cycles.

5. The method of claim 3, comprising executing 2 to 8 sparse-seed cycles.

6. The method of claim 1, prior to step (ii), executing 2 to 5 amplification cycles.

7. The method of claim 1, wherein step (ii) comprises contacting about 90%, about 91%, about 92%, about 93%, about 94%, about 95%, about 96%, about 97%, about 98%, or about 99% of the cleavable sites with said cleaving agent.

8. The method of claim 1, wherein said cleaving agent removes about 90%, about 91%, about 92%, about 93%, about 94%, about 95%, about 96%, about 97%, about 98%, or about 99% of the plurality of amplification products.

9. The method of claim 1, wherein after step (ii), executing 2 to 50 amplification cycles.

10. The method of claim 1, wherein step (ii) further comprises incubating said fraction of cleavable sites with said cleaving agent for about 5 seconds to about 30 minutes.

11. The method of claim 1, wherein said cleavable site comprises a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence specifically recognized by a cleaving agent.

12. The method of claim 1, wherein denaturing comprises contacting said template polynucleotide with a denaturant, wherein said denaturant is a buffered solution comprising betaine, dimethyl sulfoxide (DMSO), ethylene glycol, formamide, glycerol, guanidine thiocyanate, 4-methylmorpholine 4-oxide (NMO), or a mixture thereof.

13. The method of claim 1, wherein the template polynucleotide comprises genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA).

14. The method of claim 1, wherein the template polynucleotide comprises genomic DNA.

15. The method of claim 1, wherein the cleaving agent is a programmable endonuclease.

16. The method of claim 15, wherein the programmable endonuclease further comprises a guide oligonucleotide.

17. The method of claim 16, wherein the programmable endonuclease is a TtAgo enzyme.

16. The method of claim 1, further comprising sequencing one or more of the immobilized amplification products.

19. The method of claim 18, wherein sequencing comprises sequencing by synthesis, sequencing by binding, sequencing by ligation, or pyrosequencing.

20. The method of claim 18, wherein sequencing comprises sequencing by synthesis.
